(19) United States
(12) Patent Application Publication
(10) Pub. No.: US 2025/0075218 A1
(43) Pub. Date: Mar. 6, 2025

(54) HIF1A OR GLUT1 INHIBITION IN INFLAMMATORY SKIN DISEASES

(71) Applicant: New York University, New York, NY (US)

(72) Inventors: Shruti NAIK, New York, NY (US); Ipsita SUBUDHI, New York, NY (US); Piotr KONIECZNY, New York, NY (US)

(73) Assignee: New York University, New York, NY (US)

(21) Appl. No.: 18/817,602

(22) Filed: Aug. 28, 2024

Related U.S. Application Data
(60) Provisional application No. 63/534,937, filed on Aug. 28, 2023.
(60) Provisional application No. 63/540,794, filed on Sep. 27, 2023.

(51) Int. Cl. C12N 15/113; A61K 31/05; A61K 31/12; A61K 31/122; A61K 31/167; A61K 31/198; A61K 31/352; A61K 31/357; A61K 31/365; A61K 31/366; A61K 31/4045; A61K 31/427; A61K 31/436; A61K 31/472; A61K 31/4745; A61K 31/495; A61K 31/496; A61K 31/519; A61K 31/522; A61K 31/553; A61K 31/566; A61K 31/57; A61K 31/573; A61K 31/593; A61K 31/66; A61K 35/04; A61K 38/13; A61P 17/06

(52) U.S. Cl. CPC C12N 15/1138; A61K 31/05; A61K 31/12; A61K 31/122; A61K 31/167; A61K 31/198; A61K 31/352; A61K 31/357; A61K 31/365; A61K 31/366; A61K 31/4045; A61K 31/427; A61K 31/436; A61K 31/472; A61K 31/4745; A61K 31/495; A61K 31/496; A61K 31/519; A61K 31/522; A61K 31/553; A61K 31/566; A61K 31/57; A61K 31/573; A61K 31/593; A61K 31/66; A61K 35/04; A61K 38/13; A61P 17/06; C12N 2310/11; C12N 2310/12; C12N 2310/14; C12N 2310/141; C12N 2310/531

(57) ABSTRACT

The present disclosure provides methods of treating an inflammatory skin disease (e.g., psoriasis) in a subject by administering an inhibitor of Hypoxia Inducible Factor 1α (HIF1α) or an inhibitor of glucose transporter 1 (Glut1). The present disclosure also provides topical formulations comprising an inhibitor of HIF1α or an inhibitor of Glut1.

### CROSS-REFERENCE TO RELATED APPLICATIONS

This application claims priority to U.S. Provisional Patent Application No. 63/540,794, filed Sep. 27, 2023, and U.S. Provisional Patent Application No. 63/534,937, filed Aug. 28, 2023, the disclosures of which are herein incorporated by reference in their entireties.

### STATEMENT REGARDING FEDERALLY SPONSORED RESEARCH

This invention was made with government support under A1168462 and AR079173 awarded by National Institutes of Health. The government has certain rights in the invention.

### SEQUENCE LISTING

The instant application contains a Sequence Listing which has been submitted electronically in XML file format and is hereby incorporated by reference in its entirety. Said XML file copy, created on Aug. 21, 2024, is named 243735_000385_SL.xml and is 65,465 bytes in size.

### FIELD OF THE INVENTION

The present invention relates to a method of treating an inflammatory skin disease (e.g., psoriasis) in a subject by administering an inhibitor of Hypoxia Inducible Factor 1α (HIF1α) or an inhibitor of glucose transporter 1 (Glut1). The present invention also relates to topical formulations comprising an inhibitor of HIF1α or an inhibitor of Glut1.

### BACKGROUND

Inflammatory skin diseases, such as psoriasis (PsO), atopic dermatitis (AD), and hidradenitis suppurativa (HS), are driven by unchecked immune responses that profoundly disrupt homeostatic epithelial programs of proliferation and differentiation (Griffiths et al., 2021; Guenin-Mace et al., 2022; Guttman-Yassky and Krueger, 2017). At homeostasis, the epidermis is composed of functionally distinct subsets of epithelial cells, of which proliferative basal epidermal stem cells and post-mitotic suprabasal differentiated cells represent two key cell states. Long-standing clinical and histopathological data have underscored the dramatic remodeling of these homeostatic epithelial states in inflammatory disease (Lowes et al., 2007). The immunological drivers of inflammatory skin diseases have also been carefully delineated, leading to the development of immune-specific targeted therapies (Ghoreschi et al., 2021). These include inflammatory cytokines, such as IL-17, TNFα, IL-4, and IL-13, that emanate from different classes of lymphocytes and directly signal into—and pathologically remodel—the epithelium (Nograles et al., 2008; Shao et al., 2021).

More recently, single cell transcriptional profiling of PsO and AD lesions has confirmed the presence of distinct epithelial cell states in homeostasis and inflammation with cellular resolution (Cheng et al., 2018; Reynolds et al., 2021; Travis K Hughes, 2019). These cellular atlases offer a unique opportunity to decode the precise molecular programs and transcription factor(s) controlling the inflamed epithelial state. Additionally, dysfunctional epithelia produce substantial amounts of antimicrobial peptides, chemokines, and cytokines that in turn reinforce inflammation, underscoring the presence of a feed-forward immune-epithelial circuit in inflammatory disease (Schonthaler et al., 2013). Yet, how both epithelial and immune cells simultaneously fuel hyperactive states, how inflammatory signals intersect with metabolic adaptations, and the metabolic underpinnings of dysregulated immune-epithelial communique are poorly understood.

Accordingly, there is an unmet need in the art for developing novel therapies targeting immune-epithelial metabolic circuits for the treatment of inflammatory skin diseases.

### SUMMARY OF THE INVENTION

In one aspect, provided herein is a method of treating an inflammatory skin disease in a subject in need thereof, comprising administering to the subject an effective amount of an inhibitor of Hypoxia Inducible Factor 1α (HIF1α).

In some embodiments, the inflammatory skin disease is selected from psoriasis, atopic dermatitis, skin rash, hidradenitis suppurativa, actinic keratosis, seborrheic dermatitis, cutaneous lupus, and lichen planus.

In some embodiments, the inflammatory skin disease is psoriasis.

In some embodiments, the psoriasis is selected from plaque psoriasis, guttate psoriasis, pustular psoriasis, and inverse psoriasis.

In some embodiments, the inhibitor of HIF1α inhibits expression or function of HIF1α protein.

In some embodiments, the inhibitor of HIF1α is an interfering nucleic acid molecule, a ribozyme, a gene editing molecule, or a small molecule.

In some embodiments, the interfering nucleic acid molecule is an siRNA, an shRNA, a miRNA, or an antisense oligonucleotide.

In one embodiment, the interfering nucleic acid molecule is an siRNA.

In one embodiment, the siRNA comprises a nucleotide sequence

(SEQ ID NO: 35)
UGAGAGAAAUGCUUACACA,
(SEQ ID NO: 36)
GGAAAGAGAGUCAUAGAAC,
(SEQ ID NO: 37)
UUACUGAGUUGAUGGGUUA,
or
(SEQ ID NO: 38)
UUUAAUACCCUCCGAUUUA.

In some embodiments, the siRNA is chemically modified.

In one embodiment, the interfering nucleic acid molecule is an shRNA.

In one embodiment, the shRNA comprises a nucleotide sequence

(SEQ ID NO: 56)
UUAACUUGAUCCAAAGCUCUGA
(SEQ ID NO: 57)
UGAGUAAAAUCAAACACACUGU
(SEQ ID NO: 58)
UAAUAUUCAUAAAUUGAGCGGC
(SEQ ID NO: 59)
UUAUAUAUGACAGUUGCUUGAG
(SEQ ID NO: 60)
UCUGAGUAAUUCUUCACCCUGC
(SEQ ID NO: 61)
UAACUUGAUCCAAAGCUCUGAG
(SEQ ID NO: 62)
UAUAUAUGACAGUUGCUUGAGU
(SEQ ID NO: 63)
UCAGGUGAACUUUGUCUAGUGC
(SEQ ID NO: 64)
AUGAGUAAAAUCAAACACACUG
(SEQ ID NO: 65)
UAAAAUCAAACACACUGUGUCC
(SEQ ID NO: 66)
AAUAUUCAUAAAUUGAGCGGCC
(SEQ ID NO: 67)
UUUGGCAAGCAUCCUGUACUGU
(SEQ ID NO: 68)
UGAACUUUGUCUAGUGCUUCCA
(SEQ ID NO: 69)
AUAAUAUUCAUAAAUUGAGCGG
(SEQ ID NO: 70)
UAUGACAGUUGCUUGAGUUUCA
(SEQ ID NO: 71)
UUGGCAAGCAUCCUGUACUGUC
or
(SEQ ID NO: 72)
UAUAUUCCUAAAAUAAUGCUUC.

In some embodiments, the shRNA is chemically modified.

In some embodiments, the small molecule is selected from Mitoquinone mesylate, TLC-388 HCl, HS-111, IDF-11774, LW-1564, NXC-828, BAY-87-2243, Camptothecin, DFN-529, FG-2216, 2-Methoxyestradiol, AG-311, BACPTDP, BAY-97-2243, DD-001, Drupanol, GBH-1a, LS-081, Palomids, PX-478, RY-10-4, SR-16388, XL-388, and YC-1.

In one embodiment, the inhibitor of HIF1α is administered topically to an affected skin area.

In some embodiments, the inhibitor of HIF1α is administered to the suprabasal layer of the epidermis in the affected skin area.

In some embodiments, the inhibitor of HIF1α is formulated for topical administration.

In some embodiments, the inhibitor of HIF1α is formulated for topical administration to the suprabasal layer of the epidermis.

In some embodiments, the inhibitor of HIF1α is formulated in nanoparticles or liposomes.

In one embodiment, the nanoparticles or liposomes facilitate targeted delivery of the inhibitor of HIF1α to the suprabasal layer of the epidermis.

In some embodiments, the method further comprises administering to the subject an effective amount of an anti-psoriasis agent.

In some embodiments, the anti-psoriasis agent is selected from creams, ointments and other products for the skin (e.g., corticosteroids, coal tar, anthralin, vitamin D analogues, retinoids, and calcineurin inhibitors), light therapy (e.g., sunlight, artificial UVB light, and artificial UVA light), and pills and injections (e.g., retinoids, methotrexate, cyclosporine, and biologics).

In some embodiments, the subject is human.

In another aspect, provided herein is a topical formulation comprising an inhibitor of Hypoxia Inducible Factor 1α (HIF1α) and a pharmaceutically acceptable carrier or excipient.

In some embodiments, the inhibitor of HIF1α inhibits expression or function of HIF1α protein.

In some embodiments, the inhibitor of HIF1α is an interfering nucleic acid molecule, a ribozyme, a gene editing molecule, or a small molecule.

In some embodiments, the interfering nucleic acid molecule is an siRNA, an shRNA, a miRNA, or an antisense oligonucleotide.

In one embodiment, the interfering nucleic acid molecule is an siRNA.

In one embodiment, the siRNA comprises a nucleotide sequence

(SEQ ID NO: 35)
UGAGAGAAAUGCUUACACA,
(SEQ ID NO: 36)
GGAAAGAGAGUCAUAGAAC,
(SEQ ID NO: 37)
UUACUGAGUUGAUGGGUUA,
or
(SEQ ID NO: 38)
UUUAAUACCCUCCGAUUUA.

In some embodiments, the siRNA is chemically modified.

In one embodiment, the interfering nucleic acid molecule is an shRNA.

In one embodiment, the shRNA comprises a nucleotide sequence

(SEQ ID NO: 56)
UUAACUUGAUCCAAAGCUCUGA
(SEQ ID NO: 57)
UGAGUAAAAUCAAACACACUGU
(SEQ ID NO: 58)
UAAUAUUCAUAAAUUGAGCGGC
(SEQ ID NO: 59)
UUAUAUAUGACAGUUGCUUGAG
(SEQ ID NO: 60)
UCUGAGUAAUUCUUCACCCUGC
(SEQ ID NO: 61)
UAACUUGAUCCAAAGCUCUGAG
(SEQ ID NO: 62)
UAUAUAUGACAGUUGCUUGAGU
(SEQ ID NO: 63)
UCAGGUGAACUUUGUCUAGUGC
(SEQ ID NO: 64)
AUGAGUAAAAUCAAACACACUG
(SEQ ID NO: 65)
UAAAAUCAAACACACUGUGUCC
(SEQ ID NO: 66)
AAUAUUCAUAAAUUGAGCGGCC
(SEQ ID NO: 67)
UUUGGCAAGCAUCCUGUACUGU
(SEQ ID NO: 68)
UGAACUUUGUCUAGUGCUUCCA
(SEQ ID NO: 69)
AUAAUAUUCAUAAAUUGAGCGG
(SEQ ID NO: 70)
UAUGACAGUUGCUUGAGUUUCA
(SEQ ID NO: 71)
UUGGCAAGCAUCCUGUACUGUC
or
(SEQ ID NO: 72)
UAUAUUCCUAAAAUAAUGCUUC.

In some embodiments, the shRNA is chemically modified.

In some embodiments, the small molecule is selected from Mitoquinone mesylate, TLC-388 HCl, HS-111, IDF-11774, LW-1564, NXC-828, BAY-87-2243, Camptothecin, DFN-529, FG-2216, 2-Methoxyestradiol, AG-311, BACPTDP, BAY-97-2243, DD-001, Drupanol, GBH-1a, LS-081, Palomids, PX-478, RY-10-4, SR-16388, XL-388, and YC-1.

In some embodiments, the inhibitor of HIF1α is formulated for topical administration to the suprabasal layer of the epidermis.

In some embodiments, the inhibitor of HIF1α is formulated in nanoparticles or liposomes.

In some embodiments, the nanoparticles or liposomes facilitate targeted delivery of the inhibitor of HIF1α to the suprabasal layer of the epidermis.

In some embodiments, the formulation further comprises an anti-psoriasis agent. In some embodiments, the anti-psoriasis agent is selected from creams, ointments and other products for the skin (e.g., corticosteroids, coal tar, anthralin, vitamin D analogues, retinoids, and calcineurin inhibitors), light therapy (e.g., sunlight, artificial UVB light, and artificial UVA light), and pills and injections (e.g., retinoids, methotrexate, cyclosporine, and biologics).

In another aspect, provided herein is a method of treating an inflammatory skin disease in a subject in need thereof, comprising administering to the subject an effective amount of an inhibitor of glucose transporter 1 (Glut1).

In some embodiments, the inflammatory skin disease is selected from psoriasis, atopic dermatitis, skin rash, hidradenitis suppurativa, actinic keratosis, seborrheic dermatitis, cutaneous lupus, and lichen planus.

In some embodiments, the inflammatory skin disease is psoriasis.

In some embodiments, the psoriasis is selected from plaque psoriasis, guttate psoriasis, pustular psoriasis, and inverse psoriasis.

In some embodiments, the inhibitor of Glut1 inhibits expression or function of Glut1 protein.

In some embodiments, the inhibitor of Glut1 is an interfering nucleic acid molecule, a ribozyme, a gene editing molecule, or a small molecule.

In some embodiments, the interfering nucleic acid molecule is an siRNA, an shRNA, a miRNA, or an antisense oligonucleotide.

In one embodiment, the interfering nucleic acid molecule is an siRNA.

In one embodiment, the siRNA comprises a nucleotide sequence

(SEQ ID NO: 35)
UGAGAGAAAUGCUUACACA,
(SEQ ID NO: 36)
GGAAAGAGAGUCAUAGAAC,
(SEQ ID NO: 37)
UUACUGAGUUGAUGGGUUA,
or
(SEQ ID NO: 38)
UUUAAUACCCUCCGAUUUA.

In some embodiments, the siRNA is chemically modified.

In one embodiment, the interfering nucleic acid molecule is an shRNA.

In one embodiment, the shRNA comprises a nucleotide sequence

(SEQ ID NO: 56)
UUAACUUGAUCCAAAGCUCUGA
(SEQ ID NO: 57)
UGAGUAAAAUCAAACACACUGU
(SEQ ID NO: 58)
UAAUAUUCAUAAAUUGAGCGGC
(SEQ ID NO: 59)
UUAUAUAUGACAGUUGCUUGAG
(SEQ ID NO: 60)
UCUGAGUAAUUCUUCACCCUGC
(SEQ ID NO: 61)
UAACUUGAUCCAAAGCUCUGAG
(SEQ ID NO: 62)
UAUAUAUGACAGUUGCUUGAGU
(SEQ ID NO: 63)
UCAGGUGAACUUUGUCUAGUGC
(SEQ ID NO: 64)
AUGAGUAAAAUCAAACACACUG
(SEQ ID NO: 65)
UAAAAUCAAACACACUGUGUCC
(SEQ ID NO: 66)
AAUAUUCAUAAAUUGAGCGGCC
(SEQ ID NO: 67)
UUUGGCAAGCAUCCUGUACUGU
(SEQ ID NO: 68)
UGAACUUUGUCUAGUGCUUCCA
(SEQ ID NO: 69)
AUAAUAUUCAUAAAUUGAGCGG
(SEQ ID NO: 70)
UAUGACAGUUGCUUGAGUUUCA
(SEQ ID NO: 71)
UUGGCAAGCAUCCUGUACUGUC
or
(SEQ ID NO: 72)
UAUAUUCCUAAAAUAAUGCUUC.

In some embodiments, the shRNA is chemically modified.

In some embodiments, the small molecule is selected from a flavone, a flavonoid, an isoflavone, genistein, myricetin, quercetin, morin, rhamnetin, isorhamnetin, biochanin A, a lavendustin, a tyrphostin, a tyrosine-kinase inhibitor, methyl 2,5-dihydroxycinnamate, gossypol, a methylxanthine, pentoxifylline, caffeine, theophylline, phloretin, a polyphenol, resveratrol, NDGA, kaempferol, curcumin, cytochalasin B, STF-31, WZB117, BAY-876, CG-5, fasentin, apigenin, trehalose, silibinin, ritonavir, MC-4, naringenin, isoquercetin, xanthohumol, bezielle, (+)-cryptocaryone, melatonin, PUG-1, a chromopynone, rapaglutin A, EF24, a ketoxime, a polyphenolic ester, a pyrazolo-pyrimidine, a quinazoline, and a phenylalanine amide.

In one embodiment, the inhibitor of Glut1 is administered topically to an affected skin area.

In some embodiments, the inhibitor of Glut1 is administered to the basal layer of the epidermis in the affected skin area.

In some embodiments, the inhibitor of Glut1 is formulated for topical administration.

In some embodiments, the inhibitor of Glut1 is formulated for topical administration to the basal layer of the epidermis.

In some embodiments, the inhibitor of Glut1 is formulated in nanoparticles or liposomes.

In one embodiment, the nanoparticles or liposomes facilitate targeted delivery of the inhibitor of Glut1 to the basal layer of the epidermis.

In some embodiments, the method further comprises administering to the subject an effective amount of an anti-psoriasis agent.

In some embodiments, the anti-psoriasis agent is selected from creams, ointments and other products for the skin (e.g., corticosteroids, coal tar, anthralin, vitamin D analogues, retinoids, and calcineurin inhibitors), light therapy (e.g., sunlight, artificial UVB light, and artificial UVA light), and pills and injections (e.g., retinoids, methotrexate, cyclosporine, and biologics).

In some embodiments, the subject is human.

In another aspect, provided herein is a topical formulation comprising an inhibitor of glucose transporter 1 (Glut1) and a pharmaceutically acceptable carrier or excipient.

In some embodiments, the inhibitor of Glut1 inhibits expression or function of Glut1 protein.

In some embodiments, the inhibitor of Glut1 is an interfering nucleic acid molecule, a ribozyme, a gene editing molecule, or a small molecule.

In some embodiments, the interfering nucleic acid molecule is an siRNA, an shRNA, a miRNA, or an antisense oligonucleotide.

In one embodiment, the interfering nucleic acid molecule is an siRNA.

In one embodiment, the siRNA comprises a nucleotide sequence

(SEQ ID NO: 35)
UGAGAGAAAUGCUUACACA,
(SEQ ID NO: 36)
GGAAAGAGAGUCAUAGAAC,
(SEQ ID NO: 37)
UUACUGAGUUGAUGGGUUA,
or
(SEQ ID NO: 38)
UUUAAUACCCUCCGAUUUA.

In some embodiments, the siRNA is chemically modified.

In one embodiment, the interfering nucleic acid molecule is an shRNA.

In one embodiment, the shRNA comprises a nucleotide sequence

(SEQ ID NO: 56)
UUAACUUGAUCCAAAGCUCUGA
(SEQ ID NO: 57)
UGAGUAAAAUCAAACACACUGU
(SEQ ID NO: 58)
UAAUAUUCAUAAAUUGAGCGGC
(SEQ ID NO: 59)
UUAUAUAUGACAGUUGCUUGAG
(SEQ ID NO: 60)
UCUGAGUAAUUCUUCACCCUGC
(SEQ ID NO: 61)
UAACUUGAUCCAAAGCUCUGAG
(SEQ ID NO: 62)
UAUAUAUGACAGUUGCUUGAGU
(SEQ ID NO: 63)
UCAGGUGAACUUUGUCUAGUGC
(SEQ ID NO: 64)
AUGAGUAAAAUCAAACACACUG
(SEQ ID NO: 65)
UAAAAUCAAACACACUGUGUCC
(SEQ ID NO: 66)
AAUAUUCAUAAAUUGAGCGGCC
(SEQ ID NO: 67)
UUUGGCAAGCAUCCUGUACUGU
(SEQ ID NO: 68)
UGAACUUUGUCUAGUGCUUCCA
(SEQ ID NO: 69)
AUAAUAUUCAUAAAUUGAGCGG
(SEQ ID NO: 70)
UAUGACAGUUGCUUGAGUUUCA
(SEQ ID NO: 71)
UUGGCAAGCAUCCUGUACUGUC
or
(SEQ ID NO: 72)
UAUAUUCCUAAAAUAAUGCUUC.

In some embodiments, the shRNA is chemically modified.

In some embodiments, the small molecule is selected from a flavone, a flavonoid, an isoflavone, genistein, myricetin, quercetin, morin, rhamnetin, isorhamnetin, biochanin A, a lavendustin, a tyrphostin, a tyrosine-kinase inhibitor, methyl 2,5-dihydroxycinnamate, gossypol, a methylxanthine, pentoxifylline, caffeine, theophylline, phloretin, a polyphenol, resveratrol, NDGA, kaempferol, curcumin, cytochalasin B, STF-31, WZB117, BAY-876, CG-5, fasentin, apigenin, trehalose, silibinin, ritonavir, MC-4, naringenin, isoquercetin, xanthohumol, bezielle, (+)-cryptocaryone, melatonin, PUG-1, a chromopynone, rapaglutin A, EF24, a ketoxime, a polyphenolic ester, a pyrazolo-pyrimidine, a quinazoline, and a phenylalanine amide.

In some embodiments, the inhibitor of Glut1 is formulated for topical administration to the basal layer of the epidermis.

In some embodiments, the inhibitor of Glut1 is formulated in nanoparticles or liposomes.

In some embodiments, the nanoparticles or liposomes facilitate targeted delivery of the inhibitor of Glut1 to the basal layer of the epidermis.

In some embodiments, the formulation further comprises an anti-psoriasis agent. In some embodiments, the anti-psoriasis agent is selected from creams, ointments and other products for the skin (e.g., corticosteroids, coal tar, anthralin, vitamin D analogues, retinoids, and calcineurin inhibitors), light therapy (e.g., sunlight, artificial UVB light, and artificial UVA light), and pills and injections (e.g., retinoids, methotrexate, cyclosporine, and biologics).

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

FIGS. 1A-1E show HIF1α demarcates a dysfunctional epithelial state in human inflammatory skin disease. FIG. 1A shows UMAP visualization of single cell RNA-seq data from skin samples (Healthy, n=53844 cells; Psoriasis (PsO), n=51093 cells; Atopic dermatitis (AD), n=32017 cells). Differentiated 2 cells are demarked with dashed lines. FIG. 1B shows bar charts showing proportions of epithelial cell states in healthy, PsO, and AD skin. FIG. 1C shows on the left, Kyoto Encyclopedia of Genes and Genomes (KEGG) pathway analyses of differentially expressed genes in differentiated cluster 2 that is uniquely enriched in inflammation and on the right, upregulated genes are shown for specific pathways of interest.

FIG. 1D shows representative spatial transcriptomics (ST) plots of healthy, PsO lesional, and PsO non-lesional skin with corresponding cluster annotation and feature plots of HIF1A (GEO).

FIG. 1E shows epidermal nuclear HIF1α (green) is expressed in PsO, AD, and hidradenitis suppurativa lesional skin compared to healthy skin; HIF1α is predominantly expressed in suprabasal epidermis and can be distinguished based on punctate nuclear expression (small round circles). Keratin (KRT) 5 (red) and DAPI nuclei (blue) are also shown. Dashed white lines demarcate the dermo-epithelial borders. Asterisks denote background signal. Scale bars, 100 μm.

FIGS. 2A-2E show concomitant enrichment of Type 17 immunity and epidermal HIF1α in PsO lesions. FIG. 2A shows lesional PsO epidermis has higher activation of A1: epithelial Keratin (KRT) 5⁺ (red) HIF1α (green) (above dashed line) as compared to non-lesional skin. A2: RORγt⁺ (magenta) and CD3⁺ (yellow) T cells (below dashed line) are also enriched. DAPI nuclei (blue) are also shown. FIG. 2B shows quantification of activated epidermal KRT5+HIF1α+ cells per 100 μm of epidermis from FIG. 2A and regression plot of epidermal HIF1α+ cells and epidermal thickness (n=10 patients). FIG. 2C shows quantification of RORγt⁺ CD3⁺ cells per 100 μm of epidermis from (A) (n=10 patients). FIG. 2D shows a regression plot of epidermal HIF1α+ cells and CD3⁺ cell counts (n=10 patients). FIG. 2E shows IL-17Receptor (R) C (white) expression in PsO non-lesional and lesional tissue overlap with suprabasal, Keratin 10⁺ differentiated epidermis and HIF1⁺ cells. Basal cells are between two dashed lines and suprabasal cells are above thinner dashed line. DAPI nuclei (blue) are also shown. In FIG. 2A and FIG. 2E, white dashed lines demarcate the dermo-epithelial borders and yellow boxes denote magnified areas. Scale bars in FIG. 2A and FIG. 2E, 100 μm. Significance was determined via student paired two-tailed T Test; plots in FIG. 2B and FIG. 2C display mean+/−SEM, and each data point represents one patient.

FIGS. 3A-3F show normalization of HIF1α expression in therapy-responsive PsO patients. FIG. 3A shows a schematic of spatial transcriptomics (ST) study workflow. FIG. 3B shows a dot plot of gene expression in lesional (L) and non-lesional (NL) PsO samples pre- and post-treatment with secukinumab, depicting the frequency of cells expressing gene (percentage) and average expression per cluster (average). FIG. 3C shows following treatment with etanercept (anti-TNFα treatment), post-treated lesional (PT) PsO epidermis has lower expression of nuclear epidermal HIF1α (green) (A1, above dashed line) as compared to pre-treated lesional skin. KRT5⁺ (red), RORγt⁺ (magenta), CD3⁺ (yellow), and DAPI nuclei (blue) were shown. Dashed white lines demarcate the dermo-epithelial borders and yellow boxes denote magnified areas. Scale bars, 100 μm. Quantification of (FIG. 3D) epidermal thickness, (FIG. 3E) CD3⁺, and (FIG. 3F) activated epidermal HIF1α cells per 100 μm of epidermis from FIG. 3C (n=10 patients, N=1). Significance was determined via student paired two-tailed T Test; plots display mean+/−SEM, and each data point represents one patient.

FIGS. 4A-4G show loss of metabolic and epidermal differentiation genes following ex vivo HIF1α inhibition in human PsO lesions. FIG. 4A shows a schematic of ex vivo skin culture and treatment modalities from human PsO lesions. FIG. 4B shows volcano plots of differentially expressed genes (marked in red) between PsO+HIF1A inhibitor (inh) vs PsO+vehicle treatment (veh) and PsO+standard of care (SoC) vs PsO+veh. FIG. 4C shows number of downregulated genes in PsO+HIF1A inh and PsO+SoC vs PsO+veh. FIG. 4D shows top 25 genes downregulated in PsO+HIF1A inh as compared to healthy skin (HS) and PsO skin treatment with veh, HIF1A inh, or SoC. Scale underneath heatmap defines upregulated and downregulated genes. FIG. 4E shows a heatmap of selected genes relating to metabolism/glycolysis and epidermal development/differentiation that are downregulated in PsO+HIF1A inh as compared to PsO+veh. Scales on the right of heatmaps define upregulated and downregulated genes. FIG. 4F shows estimated marginal means for the GSVA scores of gene signatures that were downregulated in PsO+HIF1A inh (vs PsO+veh) at baseline and after 1, 4, and 12 weeks of anti-IL17 (Secukinumab) therapy or Placebo. FIG. 4G shows Secukinumab-induced changes in gene signatures that were downregulated in PsO+HIF1A inh (vs PsO+veh) in responders (R) and non-responders (NR) at 12 weeks. Data points and error bars indicate mean and SEM of the relative enrichment of DEGs; p-values below error bars represent the significance level in the change from baseline within each group at each time point, while p-values at the top indicate that treatment changes over time are significantly different between treatment groups (FIG. 4F) or R vs NR (FIG. 4G).

FIGS. 5A-5N show epidermal HIF1α is necessary for murine PsO pathology. FIG. 5A shows a schematic of imiquimod (IMQ) PsO murine model. FIG. 5B shows expression of epidermal (Keratin (K) 14, red) nuclear HIF1α (green) (identified by small round punctate staining above dashed lined) is induced following IMQ treatment. DAPI nuclei (blue) were also shown. Both (FIG. 5C) epidermal thickness and (FIG. 5D) K14⁺HIF1α⁺ cells increase following IMQ treatment. FIG. 5E shows K14⁺HIF1α⁺ cells correlate with epidermal thickness (n≥7, N=3). FIG. 5F shows a schematic of BAY 87-2243 (HIF1α inhibitor (Inh)) treatment with IMQ PsO model. Following concurrent application of BAY 87-2243 (HIF1α inhibitor, Inh) or vehicle (Veh) with IMQ, there is diminished epidermal thickness (Keratin (K) 14, red). HIF1α (green) (identified by small round punctate staining above dashed lined) and DAPI nuclei (blue) (n≥6, N=2) were shown. Quantification of (FIG. 5G) epidermal thickness, (FIG. 5H) Ki67, and (FIG. 5I) K6 thickness was shown. FIG. 5J shows following three days of IMQ, Hif1a^{EKO} mice have diminished epidermal thickness and proliferation (EdU) compared to controls (WT). EdU expression can be identified by small round punctate nuclear staining above dashed lined. EdU (white), K14 (red), and DAPI nuclei (blue) (n=7, N=3) were shown. FIG. 5K shows quantification of epidermal thickness and EdU⁺K14⁺ cells. FIG. 5L shows Hif1a^{EKO} mice have diminished Keratin (K) 6 expression (red). DAPI (blue) were shown. K6 was shown as a layer of cells above dashed line. FIG. 5M shows following five days of rmIL-23 injections, mice lacking epithelial HIF1α have diminished epidermal thickness and proliferation as compared to WT mice. EdU expression was identified by small round punctate nuclear staining above dashed line. EdU (white), K14 (red), and DAPI nuclei (blue) (n=6-7, N=2) were shown. FIG. 5N shows quantification of epidermal thickness and EdU⁺K14⁺ cells. In FIG. 5B, FIG. 5F, FIG. 5J, FIG. 5L, and FIG. 5M, white dashed lines demarcate the dermo-epithelial borders. Scale bars in FIG. 5B, FIG. 5F, FIG. 5J, FIG. 5L, and FIG. 5M, 100 μm. Significance was determined via student unpaired two-tailed T Test; plots display mean+/−SEM.

FIGS. 6A-6O show glycolysis fuels epithelial pathology and, its end product, lactate drives the inflammatory response. FIG. 6A shows volcano plot of differentially expressed genes (marked in red) between WT and Hif1a^{EKO} back skin treated for three days with IMQ (n=7, N=3). FIG. 6B shows a heatmap of selected genes relating to metabolic activity, epidermis, and immunity. Scale bars were shown on top of heatmap. Following three days of IMQ, Hif1a^{EKO} mice have diminished expression of (FIG. 6C) Glut1 (quantification in FIG. 6D) depicted by pseudocolor fire images as layer of cells above dashed line and staining quantifications (n=7, N=3). FIG. 6E shows increased expression of differentiation and metabolic genes following rmIL17A treatment of epidermal organoids (normalized to control) (n=7, N=2). FIG. 6F shows mice lacking epithelial Glut1 (Slc2a1^{EKO}) have diminished epidermal thickness (Keratin (K) 14, red) and proliferation (EdU, white) as compared to WT mice. EdU expression was identified by small round punctate nuclear staining above dashed line. FIG. 6G shows Sle2a1^{EKO} mice treated with IMQ have a decrease in dermal CD3⁺ cells as compared to WT (n=5, N=2). FIG. 6H shows reduction of dermal CD3⁺ cells (red) following concurrent application of BAY 87-2243 (HIF1α inhibitor, Inh) and IMQ (n=6, N=2). Arrows pointed to CD3⁺ cells in the images. FIG. 6I shows reduction of dermal CD3⁺ cells (red) in IMQ-treated Hif1a^{EKO} skin as compared to WT (n=6, N=3). FIG. 6J shows greater lactate expression in IL-17A-treated epithelial organoids than unstimulated control organoids (n=5, N=2). FIG. 6K shows schematic of lactate secretion assessment from ex vivo culture supernatant. Decrease in lactate secretion in IMQ-treated Slc2a1^{EKO} skin as compared to WT skin (n≥3, N=2). FIG. 6L shows schematic of LDH-A or MCT1/4 inhibitor treatment in IMQ model. Following concurrent application of sodium oxamate (LDH-A inh) or MCT1/MCT4 inhibitor (inh) with IMQ, there is diminished epidermal thickness and (FIG. 6M) dermal CD3⁺cells as compared to vehicle (veh) controls (n>6, N=3). FIG. 6N shows when LDH-A or MCT1/4 inhibitor treatment begins on day 3 of IMQ application and is concurrently applied, there is a similar decrease in dermal CD3⁺ cells as compared to vehicle (veh) control (n>6, N=3). FIG. 6O shows a schematic summarizing the findings of the study.

FIGS. 7A-7G show enrichment of Type 17 immunity and epidermal HIF1α in human inflammatory skin diseases. FIG. 7A shows UMAP visualization of combined samples (Healthy, n=53844 cells; Psoriasis (PsO), n=51093 cells; Atopic dermatitis (AD), n=32017 cells) (Reynolds et al, 2021) as per FIG. 1A. Differentiated 2 cells were marked with outline. FIG. 7B shows a dot plot of gene expression in each cluster depicting the frequency of cells expressing a given gene (percentage) and average expression per cluster (average). FIG. 7C shows Kyoto Encyclopedia of Genes and Genomes (KEGG) pathway analyses of differentially expressed genes in ST cluster 4, as denoted by FIG. 1D. Quantification of (FIG. 7D) epidermal thickness and (FIG. 7E) CD3⁺ cells per 100 μm of epidermis from FIG. 2A (n=10 patients) was shown. FIG. 7F shows epidermal HIF1α⁺ (green) cells in psoriasis (PsO), atopic dermatitis (AD), and hidradenitis suppurativa (HS) are primarily localized in the suprabasal epidermis. HIF1α expression was identified by small round punctate staining above dashed line emphasized in A2, whereas basal cells were shown in A1. Keratin (KRT) 5 (red) and DAPI nuclei (blue) were shown. Dashed white lines demarcate the dermo-epithelial borders and yellow boxes denote magnified areas. Asterisks denote background signal. Scale bars, 100 μm. FIG. 7G shows quantification of IL17RC⁺ HIF1α⁺ cells in psoriatic nonlesional (NL) and lesional (L) tissue.

FIGS. 8A-8I show loss of IL17RA/RC and HIF1α expression in therapy-responsive PsO patients. FIG. 8A shows PsO lesion pre- and post-treatment with secukinumab with full resolution of disease (PASI 100). FIG. 8B shows UMAP visualization of 779 spots colored by cluster and obtained from pre- and post-secukinumab treatment, lesional and non-lesional biopsies. FIG. 8C shows bar charts showing proportions of clusters in pre-treated and post-treated non-lesional (NL) and lesional (L) skin. FIG. 8D shows a heatmap of top marker genes in corresponding ST clusters from FIG. 7B. Lines at the bottom marked top genes per cluster from left to right. FIG. 8E shows a heatmap of additional top marker genes in corresponding ST clusters from FIG. 7B. Lines at the bottom marked top genes per cluster from left to right. FIG. 8F shows ST plots of pre-treated and post-treated non-lesional and lesional skin colored by cluster. FIG. 8G shows a dot plot of ACTB expression in each treatment group depicting the frequency of cells expressing gene (percentage) and average expression per cluster (average) for control in ST data. FIG. 8H shows representative histological images and corresponding spatial gene expression plots of HIF1A, IL17Receptor (R) A, and IL17RC (|log 2(FC)|≥1.5 and FDR<0.05) in PsO lesional samples pre- and post-treatment with secukinumab. FIG. 8I shows changes in PASI score, KRT5⁺HIF1α⁺ cells, epidermal thickness, and CD3⁺ cells in lesional (L) and post-etanercept (TNFα) treated (PT) lesions, as per therapy-responsive (PASI75) status.

FIGS. 9A-9D show differential gene expression in lesional biopsies following treatment with HIF1α inhibitor or standard of care. FIG. 9A shows comparable expression of apoptosis genes between healthy and PsO lesional skin cultures treated with vehicle (Veh), BAY 87-2243 (HIF1α inhibitor, BAY), or standard of care (calcipotriol and betamethasone dipropionate, SoC). FIG. 9B shows volcano plots of differentially expressed genes (marked in red) between healthy skin (HS)+HIF1A inhibitor (inh) vs HS+vehicle treatment (veh) and PsO+standard of care (SoC) vs HS+veh. FIG. 9C shows number of total genes in PsO+HIF1A inh and PsO+SoC vs PsO+veh. FIG. 9D shows top 50 genes upregulated in PsO+HIF1A inh as compared to healthy skin (HS) and PsO skin treatment with veh, HIF1α inh, or SoC. Scale bar to the right of the heatmap indicates upregulated and downregulated signal.

FIGS. 10A-10D show differential gene expression in lesional biopsies and murine models following treatment with HIF1α inhibitor. FIG. 10A shows estimated marginal means for the GSVA scores of upregulated gene signatures in PsO+HIF1A inh (vs PsO+veh) at baseline and after 1, 4, and 12 weeks of anti-IL17 (Secukinumab) therapy or Placebo. FIG. 10B shows Secukinumab-induced changes in upregulated gene signatures in PsO+HIF1A inh (vs PsO+veh) in responders (R) and non-responders (NR) at 12 weeks. FIG. 10C shows estimated marginal means for the GSVA scores of gene signatures downregulated (left) and upregulated (right) in imiquimod (IMQ D3)-treated Hif1a^{EKO} (vs WT) murine models at baseline and after 1, 4, and 12 weeks of anti-IL17 (Secukinumab) therapy or Placebo. FIG. 10D shows Secukinumab-induced changes in gene signatures in IMQ D3-treated HIF1α^{EKO} (vs WT) murine models in responders (R) and non-responders (NR) at 12 weeks. Data points and error bars indicate mean and 95% CI of the relative enrichment of DEGs; p-values below error bars represent the significance level in the change from baseline within each group at each time point, while p-values at the top indicate that treatment changes over time are significantly different between treatment groups (FIG. 10A and FIG. 10C) or R vs NR (FIG. 10B and FIG. 10D).

FIGS. 11A-11J show effects of HIF1α expression on murine skin pathology. FIG. 11A shows Keratin (K) 6 expression (red), discerned in cell layer above dashed line, in untreated and imiquimod (IMQ)-treated WT murine skin. DAPI (blue) were shown. FIG. 11B shows fiber probe measurement of tissue oxygen pressure (pO2) for untreated (ctr) and D6 IMQ-treated skin. FIG. 11C shows Pimonidazole staining (green) following day 0 (DO), day 3 (D3), and day 6 (D6) of IMQ. Keratin (K) 14 (red), HIF1α (white), and DAPI (blue) were shown. HIF1α was discerned as round punctate staining above dashed line. Pimonidazole (PIM) staining was defined by staining above dashed line. FIG. 11D shows IL-17ra^{-/-} mice fail to activate HIF1α following IMQ treatment. HIF1α was discerned as round punctate staining above dashed line. FIG. 11E shows global depletion of IL-17RA leads to decreased I epidermal thickness and FIG. 11F shows K14⁺HIF1α⁺ compared to wild type (WT) following IMQ (n=8, N=2). FIG. 11G shows epidermal thickness of vehicle vs HIF1α inhibitor-treated skin without IMQ. FIG. 11H shows topical application of FG-4985 (HIF1α activator, Act) on uninflamed skin for six days leads to increased K14⁺HIF1α⁺ cells but does not lead to (FIG. 11I) epidermal thickening (n=3-5, N=2). FIG. 11J shows epidermal thickness following vehicle (PBS) injection into WT and Hif1a^{EKO} ear (n=2).

FIGS. 12A-12E show HIF1α-mediated effects on inflammatory and other pathological factors. FIG. 12A shows increased expression of metabolic genes following rmIL17A treatment on WT epidermal organoids is diminished in Hif1a^{EKO} epidermal organoids (normalized to control) (n=7, N=2). FIG. 12B shows expression of differentiation genes following rmIL17A treatment of epidermal organoids (normalized to control) (n=7, N=2).

FIG. 12C shows gene expression of inflammatory mediators between IMQ-treated WT and Hif1a^{EKO} back skin. FIG. 12D shows gene expression of inflammatory mediators between healthy and PsO lesional skin cultures treated with vehicle (Veh), BAY 87-2243 (HIF1α inhibitor, BAY), or standard of care (calcipotriol and betamethasone dipropionate, SoC). FIG. 12E shows following three days of IMQ, Hif1a^{EKO} mice have diminished expression of Ndufa412, depicted by a band of cells above dashed line in pseudocolor fire images and staining quantifications (n=7, N=3).

FIGS. 13A-13E show lactate effects on inflammatory response. FIG. 13A shows IMQ-treated murine skin secretes more lactate in ex vivo culture than untreated skin. This difference is lost after treatment with 2-DG (n=5, N=2). FIG. 13B shows CD3⁺ (red) cells, marked by arrows, are diminished in mice following concurrent application of sodium oxamate (LDH-A inh) or MCT1/MCT4 inhibitor (inh) with IMQ as compared to vehicle (veh) controls. Keratin (K) 14 (green) and DAPI (blue) were shown. FIG. 13C shows when LDH-A or MCT1/4 inhibitor treatment begins on day 3 of IMQ application and is concurrently applied up to day 6, there is a decrease in epidermal thickness as compared to vehicle (veh) control (n>6, N=3). FIG. 13D shows CD3⁺ (red) cells, marked by arrows, are diminished in mice following treatment with sodium oxamate (LDH-A inh) or MCT1/MCT4 inhibitor (inh) starting at day three with IMQ as compared to vehicle (veh) controls. Keratin (K) 14 (green) and DAPI (blue) (n>6, N=3) were shown. FIG. 13E shows gene expression of inflammatory mediators between mice treated with IMQ and vehicle, LDH-A inh, or MCT1/MCT4 inh starting at day 3 of IMQ-inflammation (n=3, N=1).

FIGS. 14A-14C show the efficacy data for the HIF1α siRNAs. FIG. 14A shows the schematic overview of experiment testing topical application of HIF1α siRNAs including sequences UGAGAGAAAUGCUUACACA (SEQ ID NO: 35), GGAAAGAGAGUCAUAGAAC (SEQ ID NO: 36), UUACUGAGUUGAUGGGUUA (SEQ ID NO: 37), and UUUAAUACCCUCCGAUUUA (SEQ ID NO: 38) or control siRNA (mock) on murine skin prior to induction of imiquimod (IMQ) psoriasis murine model. FIGS. 14B-14C show representative images and quantification of epidermal thickness in HIF1α and control siRNA-treated mice. A loss of nuclear HIF1α expression (green round punctate staining of nuclei in top cell layer) in the epidermis (K14+, red) in HIF1α siRNA-treated animals correlates with reduced epidermal pathology. N=3 independent experiments with 3 mice per group.

FIG. 15 shows knockdown efficiencies of different shRNAs targeting HIF1α transcripts using a reporter cell line that measures HIF1α activity. The negative control was no amiRNA and the positive controls were shRenilla and shPten.

### DETAILED DESCRIPTION

Inflammatory epithelial conditions have long been postulated as hyperactive injury responses, co-opting similar transcriptional programs as wounds to drive epithelial dysfunction (Guenin-Mace et al., 2022). Indeed, many facets of repair are amplified in inflammatory disease including exuberant epidermal growth, hypervascularization, hyperinnervation, and immune activation. For instance, the transcription factor STAT3 is robustly expressed in wound edge proliferating epithelia and also in hyperproliferative psoriatic epithelium (Sano et al., 2005; Sano et al., 1999). A parallel factor, Hypoxia Inducible Factor 1α (HIF1α), has been identified downstream of IL-17A, which specifically controls epithelial migration in wound repair. HIFs are constitutively expressed by all metazoan cells and stabilized in the presence of hypoxia or by secondary signals such as IL-17A, TLRs, and lactate (Colegio et al., 2014; Jantsch et al., 2011; Konieczny et al., 2022; Semenza, 2003). HIF1α is specifically known to activate glycolytic programs in innate and adaptive immune cells during anti-pathogen responses and in cancer (Colegio et al., 2014; Dang et al., 2011; Jantsch et al., 2011). Unlike tissue repair or cancer, however, precisely how hyper inflamed tissues address their metabolic demands and to sustain prolonged pathological states is unclear (Eming et al., 2017). Such an understanding may reveal therapeutically targetable metabolic vulnerabilities that circumvent the global immune suppression associated with current frontline biologics.

Inflammatory skin diseases are spurred by unchecked immune-epithelial circuits. However, the specific metabolic factors involved in crosstalk and their impact on the dysregulation of these two cellular components are not well understood. Here, scRNA-seq, spatial transcriptomics, and immunofluorescence were employed across multiple disease indications to elucidate a dysfunctional epithelial state marked by HIF1α. Blocking HIF1α in human psoriasis lesions reduced disease-related gene expression. In psoriatic mouse models, epidermal specific-depletion of HIF1α or its target gene glucose transporter 1 (Glut1) ameliorated skin pathology. Glycolytic metabolism sustained epithelial hyperproliferation and differentiation, while lactate, its terminal product, was crucial for sustaining Type 17 response. Notably, inhibition of lactate dehydrogenase A or the lactate transporters MCT1/4 selectively attenuated the Type 17 response. Collectively, these findings identify therapeutically targetable metabolic vulnerabilities in inflammatory skin disease by unveiling a remarkable coordination of metabolic processes between the epithelial and immune compartments.

Here a striking enrichment of HIF1α was identified in human PsO, AD, and HS lesional skin as compared to healthy skin. Single cell and spatial transcriptomics analyses confirmed HIF1A in PsO lesional suprabasal differentiated epidermis, displaying a remarkable degree of overlap with IL-17RC expression and correlation with the presence of Th17 cells. Accordingly, resolved lesions from anti-IL-17A or anti-TNFα therapy-responsive patients showed a clear reduction in HIF1α. Inhibiting HIF1α in PsO patient biopsies reduced pathological gene expression to a greater degree than standard of care topicals, and these signatures distinguished responders from non-responders to anti-IL-17A in a clinical trial (NCT01537432) (Krueger et al., 2019). HIF1α was found to skew epithelial metabolism toward glycolysis in inflamed skin. Accordingly, loss of epidermal HIF1α or its downstream target, glucose transporter 1 (Glut1, encoded by the solute carrier family 2 member 1 (Slc2a1) gene), was sufficient to protect mice from psoriatic inflammation. HIF1α-primed glycolytic epithelia abundantly produced lactate and aggravated the cutaneous T cell inflammatory response. Surprisingly, inhibiting the lactate receptors MCT1 and MCT4 or inhibiting lactate dehydrogenase A specifically reduced the Type 17 response, revealing distinct metabolic requirements for inflamed epithelia and T cells. Overall, this data identify a distinct HIF1α-controlled epithelial state that requires glycolysis to intrinsically sustain epithelial dysfunction and potentiates the underlying inflammatory response via lactate. Appreciating the coordinated metabolic dysregulation of multiple cellular compartments in complex tissue pathologies, as shown here, has profound implications for interrupting immune-epithelial metabolic circuits to treat inflammatory epithelial diseases.

### Definitions

The term “interfering nucleic acid molecule”, as used herein, refers to a nucleic acid agent that selectively targets and inhibits the activity or expression of a product (e.g., an mRNA product) of a targeted gene (e.g., HIF1A or SLC2A1).

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or sub-clinical symptoms of the state, disorder or condition; or (2) inhibiting the state, disorder or condition, i.e., arresting, reducing, delaying or reversing the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “effective amount” refers to an amount of a compound (e.g., an inhibitor of HIF1α or an inhibitor of Glut 1) sufficient to achieve a desired effect without causing an undesirable side effect. In some cases, it may be necessary to achieve a balance between obtaining a desired effect and limiting the severity of an undesired effect. The amount of compound (e.g., an inhibitor of HIF1α or an inhibitor of Glut1) used will vary depending upon the type of active ingredient and the intended use of the composition and/or formulation of the present disclosure. The term “therapeutically effective amount” refers to the amount of a compound (e.g., an inhibitor of HIF1α or an inhibitor of Glut1) or a composition that, when administered to a subject for treating (e.g., preventing, ameliorating, or reversing) a state, disorder or condition, is sufficient to affect such treatment. The “therapeutically effective amount” will vary depending, e.g., on the compound, or analogues administered as well as the disease, its severity, and physical conditions and responsiveness of the subject to be treated.

The term “simultaneous administration” or “administer simultaneously”, as used herein, means that a first agent and second agent in a combination therapy are administered with a time separation of no more than about 15 minutes, such as no more than about any of 10, 5, or 1 minutes. When the first and second agents are administered simultaneously, the first and second agents may be contained in the same composition (e.g., a composition comprising both a first and second agent) or in separate compositions (e.g., a first agent in one composition and a second agent is contained in another composition).

As used herein, the term “sequential administration” or “administer sequentially” means that the first agent and second agent in a combination therapy are administered with a time separation of more than about 15 minutes, such as more than about any of 20, 30, 40, 50, 60, or more minutes. Either the first agent or the second agent may be administered first. The first and second agents are contained in separate compositions, which may be contained in the same or different packages or kits.

As used herein, the term “concurrent administration” or “administrated concurrently” means that the administration of the first agent and that of a second agent in a combination therapy overlap with each other.

As used herein, the terms “topical” or “topical administration”, are meant to encompass local administration of a composition as described herein, for example, as a fluid composition, to the surface of a skin or mucosal tissue of a subject without inducing any systemic effect, including parenteral, nasal, and cutaneous administration.

As used herein, a “pharmaceutical composition” refers to a composition that is pharmaceutically acceptable. As used herein, the term “pharmaceutically acceptable” refers to those compounds, compositions, materials, and/or dosage forms that are, within the scope of sound medical judgment, suitable for contact with the tissues of human beings and animals without excessive toxicity, allergic response, irritation, or other complications corresponding with a reasonable benefit/risk ratio.

As used herein, “carriers and excipients” refers to substances that are used in the formulation of the pharmaceutical compositions, and, by themselves, generally have no or little therapeutic value.

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

The term “about” or “approximately” means within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The terms “a,” “an,” and “the” do not denote a limitation of quantity, but rather denote the presence of “at least one” of the referenced item.

The term “percent sequence identity” in the context of nucleic acid sequences means the percent of residues when a first contiguous sequence is compared and aligned for maximum correspondence to a second contiguous sequence. The length of sequence identity comparison may be over a stretch of at least about nine nucleotides, usually at least about 18 nucleotides, more usually at least about 24 nucleotides, typically at least about 28 nucleotides, more typically at least about 32 nucleotides, and preferably at least about 36, 48 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA, which includes, e.g., the programs FASTA2 and FASTA3, provides alignments and percent sequence identity of the regions of the best overlap between the query and search sequences (Pearson, Methods Enzymol. 183:63-98 (1990); Pearson, Methods Mol. Biol. 132:185-219 (2000); Pearson, Methods Enzymol. 266:227-258 (1996); Pearson, J. Mol. Biol. 276:71-84 (1998); herein incorporated by reference). Unless otherwise specified, default parameters for a particular program or algorithm are used. For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference.

A reference to a nucleotide sequence encompasses its complement unless otherwise specified. Thus, a reference to a nucleic acid having a particular sequence should be understood to encompass its complementary strand, with its complementary sequence.

The term “percent sequence identity” means a ratio, expressed as a percent of the number of identical residues over the number of residues compared.

The term “substantial similarity” or “substantial sequence similarity,” when referring to a nucleic acid or fragment thereof, means that when optimally aligned with appropriate nucleotide insertions or deletions with another nucleic acid (or its complementary strand), there is nucleotide sequence identity in at least about 85%, preferably at least about 90%, 91%, 92%, 93%, 94%, 95%, 96%, 97%, 98% or 99% of the nucleotide bases, as measured by any well-known algorithm of sequence identity, such as FASTA, BLAST or Gap, as discussed above.

The practice of the present methods employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Boni-facino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ. Additional techniques are explained, e.g., in U.S. Pat. No. 7,912,698 and U.S. Patent Appl. Pub. Nos. 2011/0202322 and 2011/0307437, each of which is herein incorporated by reference in its entirety for all intended purposes.

### Methods of Treatment

In one aspect, provided herein is a method of treating an inflammatory skin disease in a subject in need thereof, comprising administering to the subject an effective amount of an inhibitor of Hypoxia Inducible Factor 1α (HIF1α).

In another aspect, provided herein is a method of treating an inflammatory skin disease in a subject in need thereof, comprising administering to the subject an effective amount of an inhibitor of glucose transporter 1 (Glut1).

In some embodiments, the inflammatory skin disease is psoriasis, atopic dermatitis, skin rash, hidradenitis suppurativa, actinic keratosis, seborrheic dermatitis, cutaneous lupus, or lichen planus.

In some embodiments, the inflammatory skin disease may be psoriasis, such as psoriasis, atopic dermatitis, skin rash, hidradenitis suppurativa, actinic keratosis, seborrheic dermatitis, cutaneous lupus, and lichen planus.

The inhibitor of HIF1α may inhibit expression or function of HIF1α protein. The inhibitor of Glut1 may inhibit expression or function of Glut1 protein.

In some embodiments, the inhibitor of HIF1α is an interfering nucleic acid molecule, a ribozyme, a gene editing molecule (e.g., CRISPR/Cas, ZFN, or TALEN), or a small molecule.

In some embodiments, the inhibitor of Glut1 is an interfering nucleic acid molecule, a ribozyme, a gene editing molecule (e.g., CRISPR/Cas, ZFN, or TALEN), or a small molecule.

Interfering nucleic acids can include a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid: oligomer heteroduplex within the target sequence (e.g., mRNA sequence). In some embodiments, the interfering nucleic acid molecule is an siRNA, an shRNA, a miRNA, or an antisense oligonucleotide.

In some embodiments, the interfering nucleic acid molecule is an siRNA, also known as short interfering RNA or silencing RNA. In some embodiments, the siRNA molecules are 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, or more base pairs in length. In some embodiments, the siRNA molecules are 8 to 40 base pairs in length, 10 to 20 base pairs in length, 10 to 30 base pairs in length, 15 to 20 base pairs in length, 19 to 23 base pairs in length, or 21 to 24 base pairs in length.

In some embodiments, an siRNA that inhibits HIF1α expression may comprise a nucleotide sequence UGAGAGAAAUGCUUACACA (SEQ ID NO: 35), GGAAAGAGAGUCAUAGAAC (SEQ ID NO: 36), UUACUGAGUUGAUGGGUUA (SEQ ID NO: 37), or UUUAAUACCCUCCGAUUUA (SEQ ID NO: 38).

In some embodiments, an siRNA that inhibits Glut1 expression may comprise a nucleotide sequence UGAGAGAAAUGCUUACACA (SEQ ID NO: 35), GGAAAGAGAGUCAUAGAAC (SEQ ID NO: 36), UUACUGAGUUGAUGGGUUA (SEQ ID NO: 37), or UUUAAUACCCUCCGAUUUA (SEQ ID NO: 38).

In some embodiments, the methods described herein further comprises administering to the subject an effective amount of one or more siRNA nucleotides.

In some embodiments, the siRNA may be chemically modified.

The siRNA may be chemically modified within the nucleic acid backbone, the ribose sugar moiety, and/or the nucleobase.

The backbone modifications may include the incorporation of phosphorothioate linkages, in which one of the non-bridging oxygen atoms is replaced with sulfur and/or a peptide nucleic acid (PNA), in which a pseudo peptide polymer backbone substitutes the standard phosphodiester backbone of the RNA, morpholino backbones (see U.S. Pat. No. 5,034,506); amide backbones (see De Mesmacker et al. Ace. Chem. Res. 1995, 28:366-374); or MMI or methylene (methylimino) backbones.

The siRNA molecule may comprise a phosphorothioate or other modified internucleotide linkage. The modified internucleotide linkages may comprise phosphorus-containing linkages. Examples of phosphorus-containing linkages include, but are not limited to, aminoalkylphosphotriesters, phosphotriesters, chiral phosphorothioates, phosphorothioates, phosphorodithioates, methyl and other alkyl phosphonates comprising 3′ alkylene phosphonates and chiral phosphonates, phosphoramidates comprising 3′-amino phosphoramidate and aminoalkylphosphoramidates, phosphinates, thionoalkylphosphonates, thionophosphoramidates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

The siRNA molecule may comprise a phosphorothioate internucleoside linkage(s) between two or more nucleotides. The siRNA molecule may comprise a phosphorothioate internucleoside linkage(s) between all nucleotides. The siRNA molecule may comprise modified internucleotide linkages at the first, second, and/or third internucleoside linkage at the 5′ or 3′ end of the siRNA molecule.

The ribose sugar modifications may include 2′-ribose substitutions (e.g., 2′-O-methyl, 2′-O-methoxyethyl, a 2′-deoxy, 2′-fluoro, 2′-O-aminopropyl, 2′-O-dimethylaminocthyl, 2′-O-dimethylaminopropyl, 2′-O-dimethylaminoethyloxyethyl, and/or 2′-O—N-methylacetamido), creation of bridged nucleic acids (e.g., locked nucleic acid (LNA), 2′,4′-constrained 2′-O-ethyl bridged nucleic acid, and/or 2′-0,4′-C-ethylene bridged nucleic acid), and/or creation of a phosphorodiamidate morpholino oligonucleotide (i.e., the five-membered ribose sugar is replaced by a six-membered morpholine ring). In some embodiments, each nucleotide of the siRNA molecule can a modified nucleotide (e.g., a 2′-modified nucleotide). In some embodiments, each nucleotide of the siRNA molecule consists of a phosphorodiamidate morpholino.

Nucleobases can be conventional bases (A, G, C, T, U), analogs thereof (e.g., modified uridines such as 5-methoxyuridine, pseudouridine, or NI-methylpseudouridine, or others); inosine; derivatives of purines or pyrimidines (e.g., N4-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, 6-O-methylguanine, 4-thio-pyrimidines, 4-amino-pyrimidines, 4-dimethylhydrazine-pyrimidines, and 4-O-alkyl-pyrimidines. Nucleic acids can include one or more “abasic” residues where the backbone includes no nitrogenous base for position(s) of the polymer.

In some embodiments, the nucleobase modifications may include a pyrimidine methylation, such as a 5-methylcytidine or a 5-methyluridine, an abasic nucleotide, or an inverted abasic residues.

An siRNA molecule described herein may comprise one or more 2′-4′ bicyclic nucleosides in which the ribose ring may comprise a bridge moiety, e.g., connecting two atoms in the ring (e.g., connecting the 2′-O atom to the 4′-C atom via an ethylene (ENA) bridge, a methylene (LNA) bridge, or a(S)-constrained ethyl (cEt) bridge).

In addition, the siRNA molecule may be modified or include nucleoside surrogates. Single-stranded regions of an siRNA molecule may be modified or include nucleoside surrogates, e.g., the unpaired region or regions of a hairpin structure, or a region which links two complementary regions, can have modifications or nucleoside surrogates. Modifications may also include those that stabilize one or more 3′- or 5′-terminus of an siRNA molecule, e.g., against exonucleases, or favor the antisense siRNA agent to enter into RNA-induced silencing complex (RISC). Modifications can include C3 (or C6, C7, C12)amino linkers, thiol linkers, carboxyl linkers, non-nucleotidic spacers (C3, C6, C9, C12, abasic, tri ethylene glycol, hexaethylene glycol), special biotin or fluorescein reagents that come as phosphoramidites and that have another DMT-protected hydroxyl group, allowing multiple couplings during RNA synthesis.

The siRNA molecule may comprise a mix of nucleosides of different kinds. For example, the siRNA molecule may comprise a mix of deoxyribonucleosides or ribonucleosides and 2′-O-methyl modified nucleosides. A siRNA described herein may comprise a mix of 2′-fluoro modified nucleosides and 2′-O-methyl modified nucleosides. An siRNA described herein may comprise a mix of 2′-4′ bicyclic nucleosides and 2′-fluoro, 2′-O-methoxyethyl, or 2′-O-methyl modified nucleosides. An siRNA described herein may comprise a mix of non-bicyclic 2′-modified nucleosides (e.g., 2′-fluoro, 2′-O-methoxyethyl, or 2′-O-methyl) and 2′-4′ bicyclic nucleosides (e.g., ENA, cEt, LNA). An siRNA described herein may comprise a mix of 2′-deoxyribonucleosides or ribonucleosides and 2′-fluoro modified nucleosides.

In some embodiments, the interfering nucleic acid molecule is an shRNA, also known as short hairpin RNA or small hairpin RNA. In some embodiments, the shRNA molecules are 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, or more base pairs in length. In some embodiments, the shRNA molecules are 8 to 40 base pairs in length, 10 to 30 base pairs in length, 20 to 30 base pairs in length, 20 to 25 base pairs in length, 21 to 25 base pairs in length, or 21 to 24 base pairs in length.

In some embodiments, an shRNA that inhibits HIF1α expression may comprise a nucleotide sequence TTAACTTGATCCAAAGCTCTGA (SEQ ID NO: 39), TGAGTAAAATCAAACACACTGT (SEQ ID NO: 40), TAATATTCATAAATTGAGCGGC (SEQ ID NO: 41), TTATATATGACAGTTGCTTGAG (SEQ ID NO: 42), TCTGAGTAATTCTTCACCCTGC (SEQ ID NO: 43), TAACTTGATCCAAAGCTCTGAG (SEQ ID NO: 44), TATATATGACAGTTGCTTGAGT (SEQ ID NO: 45), TCAGGTGAACTTTGTCTAGTGC (SEQ ID NO: 46), ATGAGTAAAATCAAACACACTG (SEQ ID NO: 47), TAAAATCAAACACACTGTGTCC (SEQ ID NO: 48), AATATTCATAAATTGAGCGGCC (SEQ ID NO: 49), TTTGGCAAGCATCCTGTACTGT (SEQ ID NO: 50), TGAACTTTGTCTAGTGCTTCCA (SEQ ID NO: 51), ATAATATTCATAAATTGAGCGG (SEQ ID NO: 52), TATGACAGTTGCTTGAGTTTCA (SEQ ID NO: 53), TTGGCAAGCATCCTGTACTGTC (SEQ ID NO: 54), or TATATTCCTAAAATAATGCTTC (SEQ ID NO: 55).

In some embodiments, an shRNA that inhibits Glut1 expression may comprise a nucleotide sequence TTAACTTGATCCAAAGCTCTGA (SEQ ID NO: 39), TGAGTAAAATCAAACACACTGT (SEQ ID NO: 40), TAATATTCATAAATTGAGCGGC (SEQ ID NO: 41), TTATATATGACAGTTGCTTGAG (SEQ ID NO: 42), TCTGAGTAATTCTTCACCCTGC (SEQ ID NO: 43), TAACTTGATCCAAAGCTCTGAG (SEQ ID NO: 44), TATATATGACAGTTGCTTGAGT (SEQ ID NO: 45), TCAGGTGAACTTTGTCTAGTGC (SEQ ID NO: 46), ATGAGTAAAATCAAACACACTG (SEQ ID NO: 47), TAAAATCAAACACACTGTGTCC (SEQ ID NO: 48), AATATTCATAAATTGAGCGGCC (SEQ ID NO: 49), TTTGGCAAGCATCCTGTACTGT (SEQ ID NO: 50), TGAACTTTGTCTAGTGCTTCCA (SEQ ID NO: 51), ATAATATTCATAAATTGAGCGG (SEQ ID NO: 52), TATGACAGTTGCTTGAGTTTCA (SEQ ID NO: 53), TTGGCAAGCATCCTGTACTGTC (SEQ ID NO: 54), or TATATTCCTAAAATAATGCTTC (SEQ ID NO: 55).

In some embodiments, the methods described herein further comprises administering to the subject an effective amount of one or more shRNA nucleotides.

In some embodiments, the shRNA may be chemically modified.

The shRNA may be chemically modified within the nucleic acid backbone, the ribose sugar moiety, and/or the nucleobase.

The backbone modifications may include the incorporation of phosphorothioate linkages, in which one of the non-bridging oxygen atoms is replaced with sulfur and/or a peptide nucleic acid (PNA), in which a pseudo peptide polymer backbone substitutes the standard phosphodiester backbone of the RNA, morpholino backbones (see U.S. Pat. No. 5,034,506); amide backbones (see De Mesmacker et al. Ace. Chem. Res. 1995, 28:366-374); or MMI or methylene (methylimino) backbones.

The shRNA molecule may comprise a phosphorothioate or other modified internucleotide linkage. The modified internucleotide linkages may comprise phosphorus-containing linkages. Examples of phosphorus-containing linkages include, but are not limited to, aminoalkylphosphotriesters, phosphotriesters, chiral phosphorothioates, phosphorothioates, phosphorodithioates, methyl and other alkyl phosphonates comprising 3′ alkylene phosphonates and chiral phosphonates, phosphoramidates comprising 3′-amino phosphoramidate and aminoalkylphosphoramidates, phosphinates, thionoalkylphosphonates, thionophosphoramidates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

The shRNA molecule may comprise a phosphorothioate internucleoside linkage(s) between two or more nucleotides. The shRNA molecule may comprise a phosphorothioate internucleoside linkage(s) between all nucleotides. The shRNA molecule may comprise modified internucleotide linkages at the first, second, and/or third internucleoside linkage at the 5′ or 3′ end of the shRNA molecule.

The ribose sugar modifications may include 2′-ribose substitutions (e.g., 2′-O-methyl, 2′-O-methoxyethyl, a 2′-deoxy, 2′-fluoro, 2′-O-aminopropyl, 2′-O-dimethylaminocthyl, 2′-O-dimethylaminopropyl, 2′-O-dimethylaminoethyloxyethyl, and/or 2′-O—N-methylacetamido), creation of bridged nucleic acids (e.g., locked nucleic acid (LNA), 2′,4′-constrained 2′-O-ethyl bridged nucleic acid, and/or 2′-0,4′-C-ethylene bridged nucleic acid), and/or creation of a phosphorodiamidate morpholino oligonucleotide (i.e., the five-membered ribose sugar is replaced by a six-membered morpholine ring). In some embodiments, each nucleotide of the shRNA molecule can a modified nucleotide (e.g., a 2′-modified nucleotide). In some embodiments, each nucleotide of the shRNA molecule consists of a phosphorodiamidate morpholino.

Nucleobases can be conventional bases (A, G, C, T, U), analogs thereof (e.g., modified uridines such as 5-methoxyuridine, pseudouridine, or NI-methylpseudouridine, or others); inosine; derivatives of purines or pyrimidines (e.g., N4-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, 6-O-methylguanine, 4-thio-pyrimidines, 4-amino-pyrimidines, 4-dimethylhydrazine-pyrimidines, and 4-O-alkyl-pyrimidines. Nucleic acids can include one or more “abasic” residues where the backbone includes no nitrogenous base for position(s) of the polymer.

In some embodiments, the nucleobase modifications may include a pyrimidine methylation, such as a 5-methylcytidine or a 5-methyluridine, an abasic nucleotide, or an inverted abasic residues.

An shRNA molecule described herein may comprise one or more 2′-4′ bicyclic nucleosides in which the ribose ring may comprise a bridge moiety, e.g., connecting two atoms in the ring (e.g., connecting the 2′-O atom to the 4′-C atom via an ethylene (ENA) bridge, a methylene (LNA) bridge, or a(S)-constrained ethyl (cEt) bridge).

In addition, the shRNA molecule may be modified or include nucleoside surrogates. Single-stranded regions of an shRNA molecule may be modified or include nucleoside surrogates, e.g., the unpaired region or regions of a hairpin structure, or a region which links two complementary regions, can have modifications or nucleoside surrogates. Modifications may also include those that stabilize one or more 3′- or 5′-terminus of an shRNA molecule or one or more hairpins of an shRNA molecule, e.g., against exonucleases or endonucleases, respectively. Modifications can include C3 (or C6, C7, C12)amino linkers, thiol linkers, carboxyl linkers, non-nucleotidic spacers (C3, C6, C9, C12, abasic, tri ethylene glycol, hexaethylene glycol), special biotin or fluorescein reagents that come as phosphoramidites and that have another DMT-protected hydroxyl group, allowing multiple couplings during RNA synthesis.

The shRNA molecule may comprise a mix of nucleosides of different kinds. For example, the shRNA molecule may comprise a mix of deoxyribonucleosides or ribonucleosides and 2′-O-methyl modified nucleosides. A shRNA described herein may comprise a mix of 2′-fluoro modified nucleosides and 2′-O-methyl modified nucleosides. An shRNA described herein may comprise a mix of 2′-4′ bicyclic nucleosides and 2′-fluoro, 2′-O-methoxyethyl, or 2′-O-methyl modified nucleosides. An shRNA described herein may comprise a mix of non-bicyclic 2′-modified nucleosides (e.g., 2′-fluoro, 2′-O-methoxyethyl, or 2′-O-methyl) and 2′-4′ bicyclic nucleosides (e.g., ENA, cEt, LNA). An shRNA described herein may comprise a mix of 2′-deoxyribonucleosides or ribonucleosides and 2′-fluoro modified nucleosides.

In some embodiments, the small molecule that inhibits HIF1α expression is selected from Mitoquinone (MitoQ) mesylate, TLC-388 HCl (Lipotecan), HS-111, IDF-11774, LW-1564, NXC-828, BAY-87-2243, Camptothecin (CPT), DFN-529 (RES 529), FG-2216, 2-Methoxyestradiol (2-ME2, 2-MeO-E2), AG-311, BACPTDP, BAY-97-2243, DD-001, Drupanol, GBH-1a, LS-081, Palomids, PX-478, RY-10-4, SR-16388, XL-388, and YC-1 (Lificiguat).

In some embodiments, the small molecule that inhibits Glut1 expression is selected from a flavone, a flavonoid, an isoflavone, genistein, myricetin, quercetin, morin, rhamnetin, isorhamnetin, biochanin A, a lavendustin, a tyrphostin, a tyrosine-kinase inhibitor, methyl 2,5-dihydroxycinnamate, gossypol, a methylxanthine, pentoxifylline, caffeine, theophylline, phloretin, a polyphenol, resveratrol, NDGA, kaempferol, curcumin, cytochalasin B, STF-31, WZB117, BAY-876, CG-5, fasentin, apigenin, trehalose, silibinin, ritonavir, MC-4, naringenin, isoquercetin, xanthohumol, bezielle, (+)-cryptocaryone, melatonin, PUG-1, a chromopynone, rapaglutin A, EF24, a ketoxime, a polyphenolic ester, a pyrazolo-pyrimidine, a quinazoline, and a phenylalanine amide.

In some embodiments, the methods described herein further comprises administering to the subject an effective amount of one or more small molecules.

The inhibitor of HIF1α may be administered topically to an affected skin area. In some embodiments, the inhibitor of HIF1α is administered to the suprabasal layer of the epidermis in the affected skin area.

The inhibitor of Glut1 may be administered topically to an affected skin area. In some embodiments, the inhibitor of Glut1 is administered to the basal layer of the epidermis in the affected skin area.

In some embodiments, the methods described herein further comprises administering to the subject an effective amount of one or more anti-psoriasis agents.

In some embodiments, the anti-psoriasis agent is selected from creams, ointments and other products for the skin (e.g., corticosteroids, coal tar, anthralin, vitamin D analogues, retinoids, and calcineurin inhibitors), light therapy (e.g., sunlight, artificial UVB light, and artificial UVA light), and pills and injections (e.g., retinoids, methotrexate, cyclosporine, and biologics), for example, as described in “Psoriasis treatment options”, Mayo Clinic, 15 Feb. 2023, which is incorporated herein by reference in its entirety.

The one or more anti-psoriasis agents may be administered simultaneously or sequentially with the inhibitor of HIF1α. The one or more anti-psoriasis agents may also be administered concurrently with the inhibitor of HIF1α.

The one or more anti-psoriasis agents may be administered simultaneously or sequentially with the inhibitor of Glut1. The one or more anti-psoriasis agents may also be administered concurrently with the inhibitor of Glut1.

### Topical Formulations

The inhibitor of HIF1α as described herein may be formulated for topical administration, for example, in liquid dosage form or semi-solid dosage form. Thus, a high level of stability of the active agent in solution has to be ensured during the processes of transport, storage, and use.

The inhibitor of Glut1 as described herein may be formulated for topical administration, for example, in liquid dosage form or semi-solid dosage form. Thus, a high level of stability of the active agent in solution has to be ensured during the processes of transport, storage, and use.

In some embodiments, the inhibitor of HIF1α (e.g., small molecules or interfering nucleic acid molecules such as siRNAs or shRNAs) is formulated in nanoparticles or liposomes. The nanoparticles or liposomes may facilitate targeted delivery of the inhibitor of HIF1α to the suprabasal layer of the epidermis.

In some embodiments, the inhibitor of Glut1 (e.g., small molecules or interfering nucleic acid molecules such as siRNAs or shRNAs) is formulated in nanoparticles or liposomes. The nanoparticles or liposomes may facilitate targeted delivery of the inhibitor of Glut1 to the basal layer of the epidermis.

In some embodiments, the methods described herein further comprises administering to the subject an effective amount of one or more siRNA nucleotides. In some embodiments, the methods described herein further comprises administering to the subject an effective amount of one or more shRNA nucleotides. In some embodiments, the methods described herein further comprises administering to the subject an effective amount of one or more small molecules.

In some embodiments, the inhibitor of HIF1α or the inhibitor of Glut1 (e.g., small molecules or interfering nucleic acid molecules such as siRNAs or shRNAs) described herein may be optionally modified and/or may use a penetration enhancing carrier system to be delivered through the stratum corneum of the skin, for example, as described in Aldawsari, et. al., Current Pharmaceutical Design, 2015, 21 and/or Roberts, et. al., Nature Reviews, Drug Discovery, 2020, 19, 673-694, each of which is incorporated herein by reference in its entirety.

The interfering nucleic acid molecules (e.g., siRNAs or shRNAs) described herein, optionally modified as described above, may be covalently conjugated to, form a complex with, and/or be encapsulated by various topical delivery-promoting moieties as described herein.

The carrier system, comprising the interfering nucleic acid molecule (e.g., siRNA or shRNA) with the topical delivery-promoting moieties, may have the ability to cross the stratum corneum to allow delivery to the skin target cells (e.g., suprabasal cells) or to the basal cells, and further, it may have the ability to improve the uptake of the interfering nucleic acid molecule (e.g., siRNA or shRNA) into the cytoplasm before degradation.

The interfering nucleic acid molecules (e.g., siRNAs or shRNAs) described herein may be conjugated to aptamers, antibodies, sugars (e.g., N-acetylgalactosamine), and/or lipids (e.g., cholesterol, α-tocopherol, and/or a long-chain fatty acid).

The interfering nucleic acid molecules (e.g., siRNAs or shRNAs) described herein may be incorporated into a lipid-based system, such as liposomes and solid lipid nanoparticles encapsulating the interfering nucleic acid molecule (e.g., siRNA or shRNA).

The liposomes may be cationic liposomes. The cationic liposomes may have a diameter of about 5 nm, about 10 nm, about 20 nm, about 25 nm, about 30 nm, about 40 nm, about 50 nm, about 60 nm, about 70 nm, about 75 nm, about 80 nm, about 90 nm, about 100 nm, about 125 nm, or about 150 nm. In one embodiment, the cationic liposomes may have a diameter of about 50 nm.

The cationic liposomes may have a diameter of about 5 nm to 150 nm, about 10 nm to 150 nm, about 20 nm to 150 nm, about 25 nm to 150 nm, about 30 nm to 150 nm, about 40 nm to 150 nm, about 50 nm to 150 nm, about 60 nm to 150 nm, about 70 nm to 150 nm, about 75 nm to 150 nm, about 80 nm to 150 nm, about 90 nm to 150 nm, about 100 nm to 150 nm, about 125 nm to 150 nm, about 5 nm to 125 nm, about 10 nm to 125 nm, about 20 nm to 125 nm, about 25 nm to 125 nm, about 30 nm to 125 nm, about 40 nm to 125 nm, about 50 nm to 125 nm, about 60 nm to 125 nm, about 70 nm to 125 nm, about 75 nm to 125 nm, about 80 nm to 125 nm, about 90 nm to 125 nm, about 100 nm to 125 nm, about 5 nm to 100 nm, about 10 nm to 100 nm, about 20 nm to 100 nm, about 25 nm to 100 nm, about 30 nm to 100 nm, about 40 nm to 100 nm, about 50 nm to 100 nm, about 60 nm to 100 nm, about 70 nm to 100 nm, about 75 nm to 100 nm, about 80 nm to 100 nm, about 90 nm to 100 nm, about 5 nm to 90 nm, about 10 nm to 90 nm, about 20 nm to 90 nm, about 25 nm to 90 nm, about 30 nm to 90 nm, about 40 nm to 90 nm, about 50 nm to 90 nm, about 60 nm to 90 nm, about 70 nm to 90 nm, about 75 nm to 90 nm, about 80 nm to 90 nm, about 5 nm to 80 nm, about 10 nm to 80 nm, about 20 nm to 80 nm, about 25 nm to 80 nm, about 30 nm to 80 nm, about 40 nm to 80 nm, about 50 nm to 80 nm, about 60 nm to 80 nm, about 70 nm to 80 nm, about 75 nm to 80 nm, about 5 nm to 75 nm, about 10 nm to 75 nm, about 20 nm to 75 nm, about 25 nm to 75 nm, about 30 nm to 75 nm, about 40 nm to 75 nm, about 50 nm to 75 nm, about 60 nm to 75 nm, about 70 nm to 75 nm, about 5 nm to 70 nm, about 10 nm to 70 nm, about 20 nm to 70 nm, about 25 nm to 70 nm, about 30 nm to 70 nm, about 40 nm to 70 nm, about 50 nm to 70 nm, about 60 nm to 70 nm, about 5 nm to 60 nm, about 10 nm to 60 nm, about 20 nm to 60 nm, about 25 nm to 60 nm, about 30 nm to 60 nm, about 40 nm to 60 nm, about 50 nm to 60 nm, about 5 nm to 50 nm, about 10 nm to 50 nm, about 20 nm to 50 nm, about 25 nm to 50 nm, about 30 nm to 50 nm, about 40 nm to 50 nm, about 5 nm to 40 nm, about 10 nm to 40 nm, about 20 nm to 40 nm, about 25 nm to 40 nm, about 30 nm to 40 nm, about 5 nm to 30 nm, about 10 nm to 30 nm, about 20 nm to 30 nm, about 25 nm to 30 nm, about 5 nm to 25 nm, about 10 nm to 25 nm, about 20 nm to 25 nm, about 5 nm to 20 nm, about 10 nm to 20 nm, or about 5 nm to 10 nm.

The solid lipid nanoparticles may comprise four components: the encapsulated therapeutic agent (i.e., the siRNA or shRNA), an emulsifier, a solid lipid, and water. The solid lipid nanoparticles may be prepared using the liquid form of the lipid(s) via an oil/water emulsion with the solid state of the lipid(s). The solid lipid nanoparticles may comprise about 0.01%, about 0.05%, about 0.1%, about 0.5%, about 1%, about 2%, about 5%, about 10%, about 15%, about 20%, about 25%, about 30%, about 40%, or about 50% w/w solid lipid in aqueous medium. In one embodiment, the solid lipid nanoparticles may comprise 0.1 to 30% w/w solid lipid in aqueous medium.

The solid lipid nanoparticles may comprise about 0.01% to 50%, about 0.05% to 50%, about 0.1% to 50%, about 0.5% to 50%, about 1% to 50%, about 2% to 50%, about 5% to 50%, about 10% to 50%, about 15% to 50%, about 20% to 50%, about 25% to 50%, about 30% to 50%, about 40% to 50%, about 0.01% to 40%, about 0.05% to 40%, about 0.1% to 40%, about 0.5% to 40%, about 1% to 40%, about 2% to 40%, about 5% to 40%, about 10% to 40%, about 15% to 40%, about 20% to 40%, about 25% to 40%, about 30% to 40%, about 0.01% to 30%, about 0.05% to 30%, about 0.1% to 30%, about 0.5% to 30%, about 1% to 30%, about 2% to 30%, about 5% to 30%, about 10% to 30%, about 15% to 30%, about 20% to 30%, about 25% to 30%, about 0.01% to 25%, about 0.05% to 25%, about 0.1% to 25%, about 0.5% to 25%, about 1% to 25%, about 2% to 25%, about 5% to 25%, about 10% to 25%, about 15% to 25%, about 20% to 25%, about 0.01% to 20%, about 0.05% to 20%, about 0.1% to 20%, about 0.5% to 20%, about 1% to 20%, about 2% to 20%, about 5% to 20%, about 10% to 20%, about 15% to 20%, about 0.01% to 15%, about 0.05% to 15%, about 0.1% to 15%, about 0.5% to 15%, about 1% to 15%, about 2% to 15%, about 5% to 15%, about 10% to 15%, about 0.01% to 10%, about 0.05% to 10%, about 0.1% to 10%, about 0.5% to 10%, about 1% to 10%, about 2% to 10%, about 5% to 10%, about 0.01% to 5%, about 0.05% to 5%, about 0.1% to 5%, about 0.5% to 5%, about 1% to 5%, about 2% to 5%, about 0.01% to 2%, about 0.05% to 2%, about 0.1% to 2%, about 0.5% to 2%, about 1% to 2%, about 0.01% to 1%, about 0.05% to 1%, about 0.1% to 1%, about 0.5% to 1%, about 0.01% to 0.5%, about 0.05% to 0.5%, about 0.1% to 0.5%, about 0.01% to 0.1%, about 0.05% to 0.1%, or about 0.01% to 0.05% w/w solid lipid in aqueous medium.

The solid lipid nanoparticles may have a size of about 10 nm, about 25 nm, about 40 nm, about 75 nm, about 150 nm, about 250 nm, about 500 nm, about 750 nm, about 1 μm, about 1.5 μm, or about 3 μm. In one embodiment, the solid lipid nanoparticles may have a size of 40 nm to 1 μm.

The solid lipid nanoparticles may have a size of about 10 nm to about 3 μm, about 25 nm to about 3 μm, about 40 nm to about 3 μm, about 75 nm to about 3 μm, about 150 nm to about 3 μm, about 250 nm to about 3 μm, about 500 nm to about 3 μm, about 750 nm to about 3 μm, about 1 μm to about 3 μm, about 1.5 μm to about 3 μm, about 10 nm to about 1.5 μm, about 25 nm to about 1.5 μm, about 40 nm to about 1.5 μm, about 75 nm to about 1.5 μm, about 150 nm to about 1.5 μm, about 250 nm to about 1.5 μm, about 500 nm to about 1.5 μm, about 750 nm to about 1.5 μm, about 1 μm to about 1.5 μm, about 10 nm to about 1 μm, about 25 nm to about 1 μm, about 40 nm to about 1 μm, about 75 nm to about 1 μm, about 150 nm to about 1 μm, about 250 nm to about 1 μm, about 500 nm to about 1 μm, about 750 nm to about 1 μm, about 10 nm to about 750 nm, about 25 nm to about 750 nm, about 40 nm to about 750 nm, about 75 nm to about 750 nm, about 150 nm to about 750 nm, about 250 nm to about 750 nm, about 500 nm to about 750 nm, about 10 nm to about 500 nm, about 25 nm to about 500 nm, about 40 nm to about 500 nm, about 75 nm to about 500 nm, about 150 nm to about 500 nm, about 250 nm to about 500 nm, about 10 nm to about 250 nm, about 25 nm to about 250 nm, about 40 nm to about 250 nm, about 75 nm to about 250 nm, about 150 nm to about 250 nm, about 10 nm to about 150 nm, about 25 nm to about 150 nm, about 40 nm to about 150 nm, about 75 nm to about 150 nm, about 10 nm to about 75 nm, about 25 nm to about 75 nm, about 40 nm to about 75 nm, about 10 nm to about 40 nm, about 25 nm to about 40 nm, or about 10 nm to about 25 nm.

The solid lipid nanoparticles may be surface modified, for example, conjugated with polyethylene glycol (PEG), peptides, or other ligands that confer cell-specific targeting.

The interfering nucleic acid molecules (e.g., siRNAs or shRNAs) described herein may be encapsulated into exosomes. The exosomes may have a diameter of about 25 nm, about 50 nm, about 75 nm, about 100 nm, about 150 nm, about 200 nm, or about 300 nm. In one embodiment, the exosomes may have a diameter of about 100 nm.

The exosomes may have a diameter of about 25 nm to about 300 nm, about 50 nm to about 300 nm, about 75 nm to about 300 nm, about 100 nm to about 300 nm, about 150 nm to about 300 nm, about 200 nm to about 300 nm, about 25 nm to about 200 nm, about 50 nm to about 200 nm, about 75 nm to about 200 nm, about 100 nm to about 200 nm, about 150 nm to about 200 nm, about 25 nm to about 150 nm, about 50 nm to about 150 nm, about 75 nm to about 150 nm, about 100 nm to about 150 nm, about 25 nm to about 100 nm, about 50 nm to about 100 nm, about 75 nm to about 100 nm, about 25 nm to about 75 nm, about 50 nm to about 75 nm, or about 25 nm to about 50 nm.

The exosomes may be surface modified, for example, conjugated with surface ligands, such as peptides or antigens.

The interfering nucleic acid molecules (e.g., siRNAs or shRNAs) described herein may be incorporated into a polymer-based system via encapsulation or complex formation. The polymer-based system may use polymers such as PLGA, cyclodextrin, and/or chitosan.

The PLGA may be modified, for example, by conjugation with polyethyleneimine. The PLGA may be in the form of microspheres.

The cyclodextrin may be modified, for example, by conjugation with an imidazole group.

The chitosan can be a chitosan derivative, such as but not limited to, chitosan aspartate, chitosan glutamate, chitosan acetate, chitosan hydrochloride, chitosan hydroxy benzotriazole, and/or chitosan thiamine pyrophosphate. The chitosan may be modified, for example, by conjugation with PEG. The chitosan complex may use a weight ratio of about 10, about 20, about 25, about 32, about 40, about 50, or about 75 between siRNA or shRNA and chitosan. In one embodiment, the chitosan complex may use a weight ratio of 32 between siRNA or shRNA and chitosan. The chitosan complex may use a weight ratio of about 10 to about 75, about 20 to about 75, about 25 to about 75, about 32 to about 75, about 40 to about 75, about 50 to about 75, about 10 to about 50, about 20 to about 50, about 25 to about 50, about 32 to about 50, about 40 to about 50, about 10 to about 40, about 20 to about 40, about 25 to about 40, about 32 to about 40, about 10 to about 32, about 20 to about 32, about 25 to about 32, about 10 to about 25, about 20 to about 25, or about 10 to about 20 between siRNA or shRNA and chitosan.

The polymer-based system may incorporate the interfering nucleic acid molecules (e.g., siRNAs or shRNAs) into single-walled carbon nanotubes, optionally linked to succinated polyethylenimine.

The interfering nucleic acid molecules (e.g., siRNAs or shRNAs) described herein may be incorporated into a nanoparticle conjugate system. The nanoparticle conjugate system may combine a lipid and polymer. The nanoparticle conjugate system may contain a nanoparticle of a hydrophobic polymer (e.g., PLGA) in the core and a combination of a hydrophilic polymer (e.g., PEG) with a lipid layer (e.g., DOPC) in the shell.

The nanoparticle conjugate system may be made of particles with a size of about 50 nm, about 100 nm, about 150 nm, about 165 nm, about 180 nm, about 200 nm, about 300 nm, or about 500 nm.

The nanoparticle conjugate system may be made of particles with a size of about 50 nm to about 500 nm, about 100 nm to about 500 nm, about 150 nm to about 500 nm, about 165 nm to about 500 nm, about 180 nm to about 500 nm, about 200 nm to about 500 nm, about 300 nm to about 500 nm, about 50 nm to about 300 nm, about 100 nm to about 300 nm, about 150 nm to about 300 nm, about 165 nm to about 300 nm, about 180 nm to about 300 nm, about 200 nm to about 300 nm, about 50 nm to about 200 nm, about 100 nm to about 200 nm, about 150 nm to about 200 nm, about 165 nm to about 200 nm, about 180 nm to about 200 nm, about 50 nm to about 180 nm, about 100 nm to about 180 nm, about 150 nm to about 180 nm, about 165 nm to about 180 nm, about 50 nm to about 165 nm, about 100 nm to about 165 nm, about 150 nm to about 165 nm, about 50 nm to about 150 nm, about 100 nm to about 150 nm, or about 50 nm to about 100 nm. In one embodiment, the nanoparticle conjugate system may be made of particles with a size of 150-180 nm.

The nanoparticle conjugate system may be a spherical nucleic acid nanoparticle conjugate. The spherical nucleic acid nanoparticle conjugate may comprise a hydrophobic core, such as gold, silica or other materials, in the center of a dense shell of oriented, covalently immobilized nucleic acid molecule (e.g., siRNA or shRNA). The spherical nucleic acid nanoparticle conjugate can be applied topically in a moisturizer or phosphate-buffered saline.

The interfering nucleic acid molecules (e.g., siRNAs or shRNAs) described herein may be incorporated into a dendrimer. Dendrimers may be made of poly(amidoamine) dendrimers, poly(propylene imine) dendrimers, carbosilane dendrimers, and/or poly(L-lysine) dendrimers.

The poly(amidoamine) dendrimers may have a core of ammonia, ethylenediamine, or cyclodextrin.

The interfering nucleic acid molecules (e.g., siRNAs or shRNAs) described herein may be incorporated into a polycation polymer, such as poly-L-lysine and/or polyethyleneimine. The poly-L-lysine may be incorporated into mesoporous silica nanoparticles to form a carrier. The polyethylenimine may exist in branched and/or linear form. The polyethyleneimine may be used in combination with siRNA or shRNA conjugated with another moiety (e.g., PEG).

The interfering nucleic acid molecules (e.g., siRNAs or shRNAs) described herein may be conjugated with or encapsulated by peptides, including cell penetrating peptides. The cell penetrating peptides may include trans-activating transcriptional activator (TAT), penetratin, PEP-1, transportan, MPG, PepFect6, RVG-9R, Xentry-KALA, and/or polymers of basic amino acids (e.g., arginine and lysine).

The cell penetrating peptides can deliver the interfering nucleic acid molecule (e.g., siRNA or shRNA) in the form of nanoparticles that may be between 70-200 nm in size or may be below 70 nm in size.

The interfering nucleic acid molecules (e.g., siRNAs) described herein may be incorporated into an Accell™ siRNA/Dharmacon-based formulation. The Accell™ siRNA/Dharmacon-based formulation may include addition of a small lipophile to the siRNA duplex or modification of the siRNA backbone.

The interfering nucleic acid molecules (e.g., shRNAs) described herein may be incorporated into an SMARTvector™ lentiviral shRNA/Dharmacon-based formulation. The SMARTvector™ lentiviral shRNA/Dharmacon-based formulation may include addition of a fluorescent reporter gene or a drug-selection marker gene.

The interfering nucleic acid molecules (e.g., siRNAs or shRNAs) described herein may be encapsulated by a nanosome, for example, a surfactant-ethanol-cholesterolosome. The surfactant-ethanol-cholesterolosome may be prepared using 1,2-dioleoyl-3-trimethylammonium-propane (DOTAP), cholesterol, sodium cholate, and ethanol.

The surfactant-ethanol-cholesterolosome-siRNA or surfactant-ethanol-cholesterolosome-shRNA complex may have a diameter of about 25 nm, about 50 nm, about 75 nm, about 100 nm, about 150 nm, about 200 nm, or about 300 nm. In one embodiment, the surfactant-ethanol-cholesterolosome-siRNA or surfactant-ethanol-cholesterolosome-shRNA complex may have a diameter of about 100 nm.

The surfactant-ethanol-cholesterolosome-siRNA or surfactant-ethanol-cholesterolosome-shRNA complex may have a diameter of about 25 nm to about 300 nm, about 50 nm to about 300 nm, about 75 nm to about 300 nm, about 100 nm to about 300 nm, about 150 nm to about 300 nm, about 200 nm to about 300 nm, about 25 nm to about 200 nm, about 50 nm to about 200 nm, about 75 nm to about 200 nm, about 100 nm to about 200 nm, about 150 nm to about 200 nm, about 25 nm to about 150 nm, about 50 nm to about 150 nm, about 75 nm to about 150 nm, about 100 nm to about 150 nm, about 25 nm to about 100 nm, about 50 nm to about 100 nm, about 75 nm to about 100 nm, about 25 nm to about 75 nm, about 50 nm to about 75 nm, or about 25 nm to about 50 nm.

The interfering nucleic acid molecules (e.g., siRNAs or shRNAs) described herein may be used in combination with a cream-emulsified CD86.

The interfering nucleic acid molecules (e.g., siRNAs or shRNAs) described herein may be encapsulated by a DNA nanostructure. The DNA nanostructure may be a size of about 5 nm, about 10 nm, about 15 nm, about 20 nm, about 30 nm, about 40 nm, about 50 nm, or about 60 nm. In one embodiment, the DNA nanostructure may be a size of about 20 nm.

The DNA nanostructure may be a size of about 5 nm to about 60 nm, about 10 nm to about 60 nm, about 15 nm to about 60 nm, about 20 nm to about 60 nm, about 30 nm to about 60 nm, about 40 nm to about 60 nm, about 50 nm to about 60 nm, about 5 nm to about 50 nm, about 10 nm to about 50 nm, about 15 nm to about 50 nm, about 20 nm to about 50 nm, about 30 nm to about 50 nm, about 40 nm to about 50 nm, about 5 nm to about 40 nm, about 10 nm to about 40 nm, about 15 nm to about 40 nm, about 20 nm to about 40 nm, about 30 nm to about 40 nm, about 5 nm to about 30 nm, about 10 nm to about 30 nm, about 15 nm to about 30 nm, about 20 nm to about 30 nm, about 5 nm to about 20 nm, about 10 nm to about 20 nm, about 15 nm to about 20 nm, about 5 nm to about 15 nm, about 10 nm to about 15 nm, or about 5 nm to about 10 nm.

The methods of siRNA delivery described herein may include a peptide enhancer, with a sequence of AC-TGSTQHQ-CG (SEQ ID NO: 73) (the “−” s indicate a disulfide bridge between the Cysteine residues at the second and tenth positions) to increase penetration of siRNA into the skin.

The inhibitors of HIF1α or the inhibitors of Glut1 of the disclosure provided herein may also be incorporated into topically applied vehicles, such as salves or ointments, which have both a soothing effect on the skin as well as a means for administering the active ingredient directly to the affected area.

The carrier in a topical formulation for the active ingredient may be either in sprayable or non-sprayable form.

Non-sprayable forms can be liquid forms, semi-solid forms, or solid forms comprising a carrier indigenous to topical application and can have a dynamic viscosity greater than that of water. Suitable formulations include, but are not limited to, solutions, pastes, lotions, oils, tinctures, gels, suspensions, gel-like solutions or gel-like suspensions, emulsions, creams, ointments, powders, liniments, salves, foams, nasal drops, and the like, or any other formulation known to a person skilled in the art. These may be optionally sterilized or mixed with auxiliary agents, e.g., preservatives, stabilizers, wetting agents, buffers, or salts for influencing osmotic pressure and the like. Vehicles for non-sprayable topical preparations can include epicutaneous, percutaneous, and transdermal patches; ointment bases, e.g., polyethylene glycol-1000 (PEG-1000); conventional creams such as HEB cream; gels; as well as petroleum jelly, such as petrolatum, wax, such as lanolin, and the like.

Also suitable for topical application are sprayable aerosol preparations wherein the active ingredient, optionally in combination with a solid or liquid inert carrier material, is packaged in a squeeze bottle or in admixture with a pressurized volatile, normally gaseous propellant. The aerosol may be a nasal spray, or the like, or any other formulation known to a person skilled in the art. The aerosol preparations can contain solvents, buffers, surfactants, perfumes, and/or antioxidants in addition to the compounds of the disclosure.

The inhibitors of HIF1α or the inhibitors of Glut1 as described herein may be formulated in a solid dosage form such as lyophilizates, which may be combined with an appropriate fluid before administration. This formulation may be applied through the nose or skin. Examples include sprays, liquids, gels, ointments, including emulsions, and aerosols.

The dosage forms as described herein, serve as carriers for the therapeutic active ingredient that is topically delivered. Examples for an appropriate route of administration are topical administration by way of the skin and mucosa, e.g., nasal mucosa, buccal tissue, vagina, rectal tissue, cornea, urethral membrane, external car lining, etc.

The compositions may be pharmaceutical compositions. The composition as described herein optionally comprises one or more pharmaceutically acceptable carriers or excipients.

Typical excipients include solvents, surfactants, antioxidants, oils, salts, metal-ions, anti-bacterial agents and other preservatives; buffering agents; chelating agents; coloring, flavoring and diluting agents; agents for adjusting tonicity; emulsifying and suspending agents; thickening agents; and other substances with pharmaceutical applications. Non-limiting examples for pharmaceutically acceptable carriers and excipients suitable for use in the compositions as described herein are antioxidants; gel-forming, specifically hydrogel-forming substances; buffering agents; surfactants and salts used for tonicifying and enhancing colloidal stability; and special ligands, such as metal ions for use in the enzymatic active area.

Antioxidants can include substances that inhibit or delay the oxidation of biologically relevant molecules, e.g., by quenching free radicals or by chelation of redox metals. Example antioxidants include methionine and cysteine.

Hydrogel-forming substances refer to substances formed when an organic polymer, either natural or synthetic, is crosslinked via covalent, ionic, or hydrogen bonds to create a three-dimensional structure that entraps or bonds with water molecules and/or activating fluid, for example, aqueous fluid. Example hydrogel-forming substances include starch, hydroxyethyl cellulose, alginate, carmellose, and Poloxamers.

Poloxamers include block copolymers of poly(ethylene oxide) and poly(propylene oxide), known as non-ionic surfactants that, in high concentrations, form aqueous gels that transition from a low to a high viscous state upon an increase in temperature, known as the process of “thermal gelation”. Poloxamers are also commonly used as surfactants in protein formulation, and thereby reduce aggregation and air-water interface interaction. An example Poloxamer is Poloxamer 188.

The composition as described herein may comprise one or more piperazine- or morpholine-containing zwitterionic buffering substance, for example, 2-(4-(2-hydroxyethyl)-1-piperazinyl)-ethane sulfonic acid (HEPES), and optionally one or more pharmaceutically acceptable carriers or excipients as defined herein. Additional examples for pharmaceutically acceptable carriers or excipients are CaCl₂), NaCl, and Arginine-HCl.

The pH value of the composition as described herein can be in the range of about pH 4 to about 8. For example, the pH value of the composition as described herein can be about 4, about 4.5, about 5, about 5.5, about 6, about 6.5, about 7, about 7.5, or about 8. The pH value of the composition as described herein can about 4 to about 8, about 4.5 to about 8, about 5 to about 8, about 5.5 to about 8, about 6 to about 8, about 6.5 to about 8, about 7 to about 8, about 7.5 to about 8, about 4 to about 7.5, about 4.5 to about 7.5, about 5 to about 7.5, about 5.5 to about 7.5, about 6 to about 7.5, about 6.5 to about 7.5, about 7 to about 7.5, about 4 to about 7, about 4.5 to about 7, about 5 to about 7, about 5.5 to about 7, about 6 to about 7, about 6.5 to about 7, about 4 to about 6.5, about 4.5 to about 6.5, about 5 to about 6.5, about 5.5 to about 6.5, about 6 to about 6.5, about 4 to about 6, about 4.5 to about 6, about 5 to about 6, about 5.5 to about 6, about 4 to about 5.5, about 4.5 to about 5.5, about 5 to about 5.5, about 4 to about 5, about 4.5 to about 5, or about 4 to about 4.5.

A thickening agent can be a substance which, when added to various blends of non-aqueous and aqueous solutions comprising the composition as described herein, increases the viscosity of said solution without substantially affecting the biological activity of the active ingredients present or the chemical and physical stability of the composition.

Gel-forming substances can be polymers that are able to form three-dimensional networks upon cross-linking and the subsequent absorption of water.

Examples of thickening and swelling agents include, but are not limited to, methyl cellulose, hydroxypropyl methylcellulose (short form: HPMC or hypromellose), hydroxypropyl cellulose, carboxymethyl cellulose, hydroxyethyl cellulose, xanthan gums, alginates, Xellan gums, pectins, Gummi arabicum, and other polysaccharide-based gel-formers or starches.

The compositions and formulations of the composition as described herein as an active ingredient are applied in an effective amount when used in prophylaxis and therapy.

In some embodiments, the formulation described herein further comprises one or more anti-psoriasis agents. In some embodiments, the anti-psoriasis agent is selected from creams, ointments and other products for the skin (e.g., corticosteroids, coal tar, anthralin, vitamin D analogues, retinoids, and calcineurin inhibitors), light therapy (e.g., sunlight, artificial UVB light, and artificial UVA light), and pills and injections (e.g., retinoids, methotrexate, cyclosporine, and biologics), for example, as described in “Psoriasis treatment options”, Mayo Clinic, 15 Feb. 2023, which is incorporated herein by reference in its entirety.

The amount of active ingredient used will vary depending upon the intended use of the composition and/or formulation; the type of active ingredient used; the age, health, and weight of the recipient; type of concurrent treatment, if any; frequency of treatment; and the nature of the effect desired.

### EXAMPLES

The following examples are provided to further describe some of the embodiments disclosed herein. The examples are intended to illustrate, not to limit, the disclosed embodiments.

### Example 1. HIF1α Marks an Aberrant Epithelial State in Human Inflammatory Skin Diseases

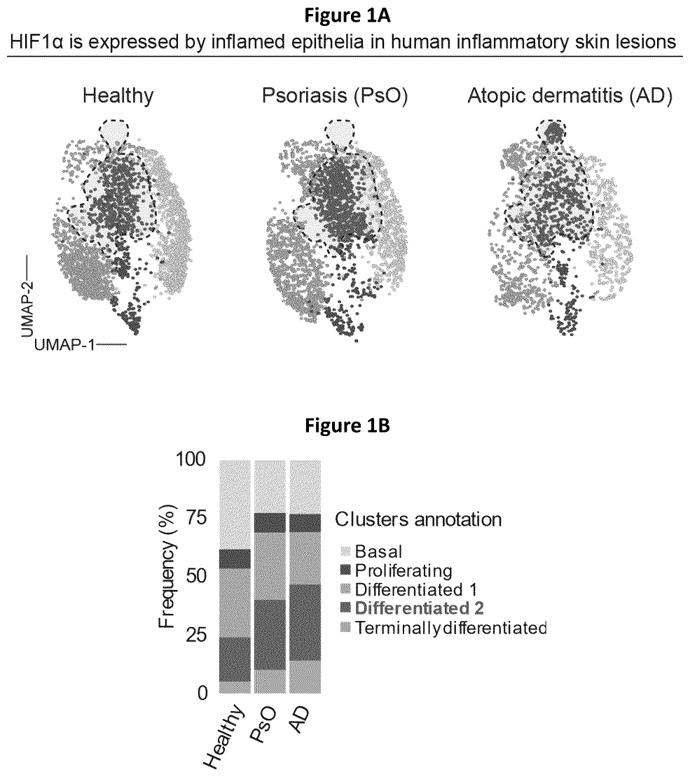

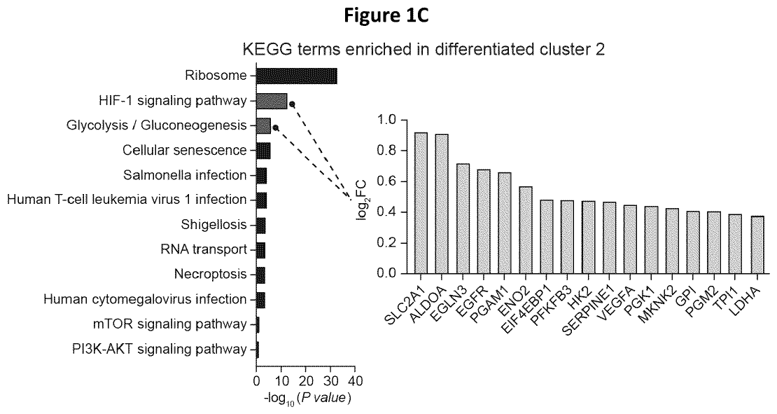

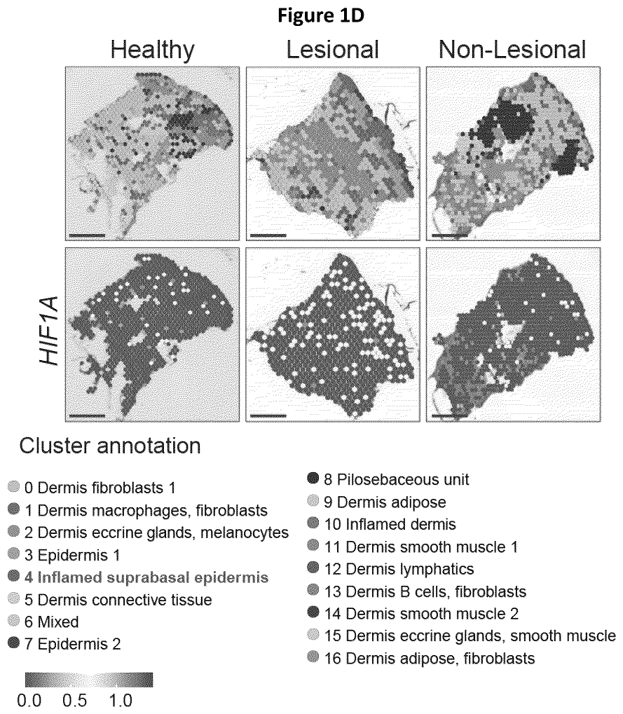

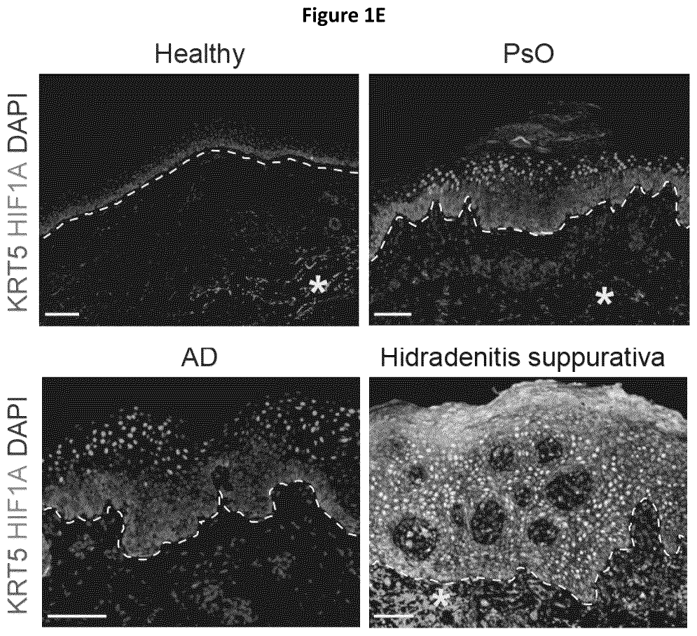

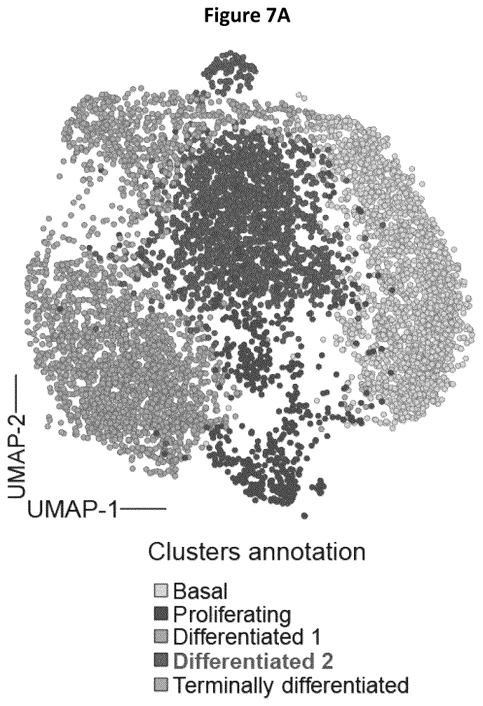

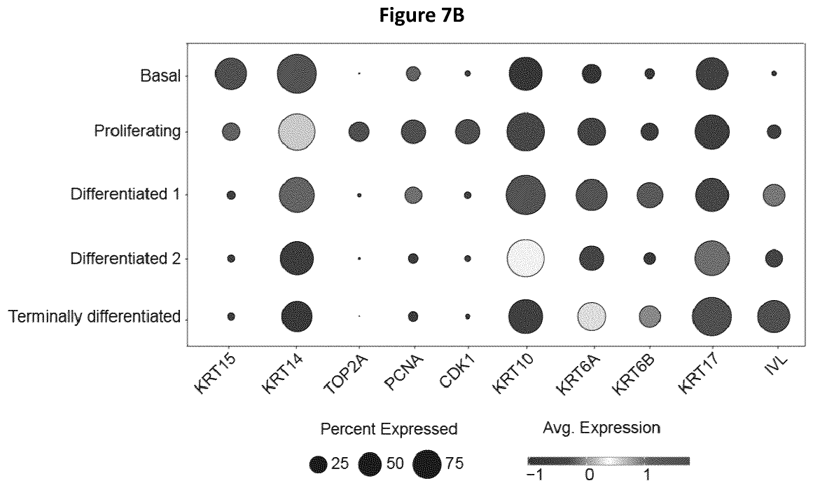

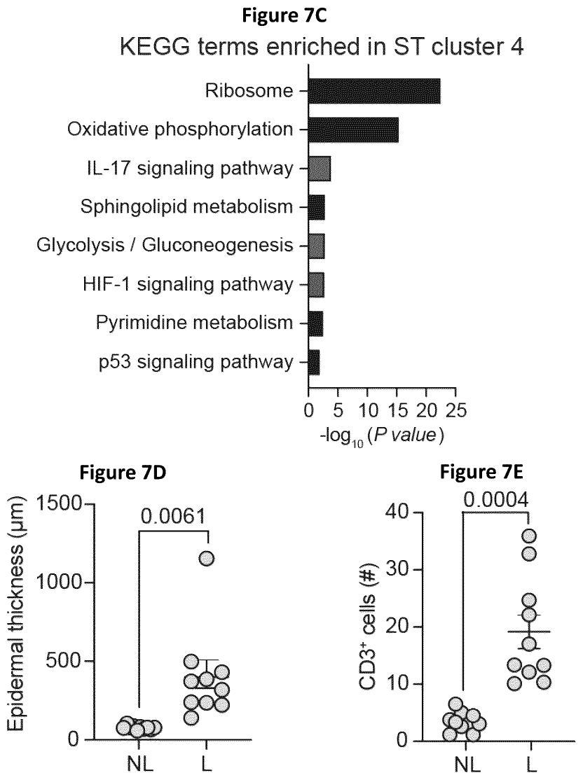

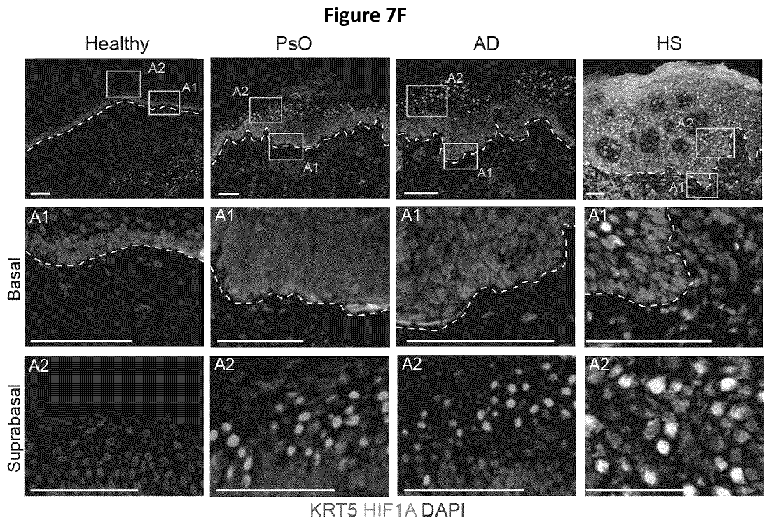

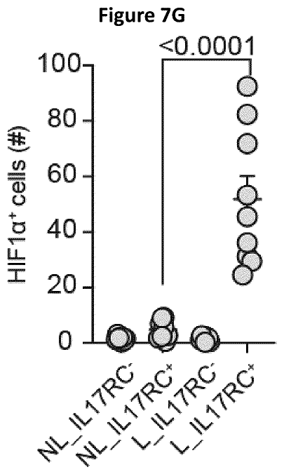

To delineate the molecular changes and master regulators of epidermal pathology in inflammatory skin disease, first publicly available single cell transcriptomics datasets (ENA-ERP116319) from the human cell atlas were analyzed. The epithelial compartment of healthy, PsO, and AD skin was of focus (FIGS. 1A and 7A). Epithelial-specific sub clustering revealed 5 different populations, including basal stem cell cluster (KRT14, KRT15), proliferating cell cluster (TOP2, PCNA, CDKN1), differentiated cluster 1 (KRT10), differentiated cell cluster 2 (KRT10, KRT17), and terminally differentiated cell cluster (IVL) (FIG. 7B). Of these, differentiated cell cluster 2 was enriched in both PsO and AD as compared to healthy (FIGS. 1A and 1B). Therefore, differentially expressed genes (FDR<0.05) were evaluated in differentiated cluster 2 relative to other clusters in healthy and inflamed epithelium. Kyoto Encyclopedia of Genes and Genomes (KEGG) pathways analysis revealed a striking enrichment of HIF1α signaling and glycolysis/gluconeogenesis genes (FIG. 1C) (Kanchisa and Goto, 2000). The genes represented in these KEGG terms included both glucose transporters and glycolytic enzymes (SLC2A1, GPI, HK2, LDHA) and other HIF1α targets involved in repair, inflammation, and cancer (VEGFA, EGFR) (Canavese et al., 2010; Dai et al., 2022).

Spatial transcriptomics (ST) is a powerful technology that allows for precise mapping of gene expression and cellular ecosystems in complex tissues (Moncada et al., 2020). Importantly, this platform captures cell types that are fragile or lack nuclei and are often lost to tissue dissociation for single cell analysis, including various differentiated epithelial populations which can be underrepresented in scRNA-seq data. A ST dataset of 25 healthy and PsO lesional and non-lesional skin samples was utilized (GSE202011) (Castillo et al., 2023). Consistent with the scRNA-seq data, the ST analysis discerned an inflammatory suprabasal epidermal population (cluster 4) enriched specifically in PsO lesional skin (FIG. 1D). Additionally, IL-17 signaling, glycolysis/gluconeogenesis, and HIF1α signaling pathways were all enriched in this PsO-specific population relative to all other clusters in healthy and PsO skin (FIG. 7C).

HIFs are constitutively expressed and degraded in the cytoplasm of all metazoan cells; however, upon their activation either by hypoxia or other secondary signals such as IL-17A, they translocate to the nucleus and enact their transcriptional function (Konieczny et al., 2022; Semenza, 2012). The prominent enrichment of HIF1A and its downstream targets in these omics data prompted study to corroborate whether HIF1α protein was activated in human inflammatory skin disease. Indeed, HIF1α was robustly expressed and localized to the nucleus of PsO lesional epidermis (FIG. 1E). This same pattern of epidermal HIF1α activation was also evident in AD and HS lesional skin. By contrast, healthy human epidermis had a notable absence of either cytoplasmic or nuclear HIF1α signal (FIG. 1E).

### Example 2. A Striking Pattern of Type 17 Immunity and Epidermal HIF1α Co-Activation in PsO

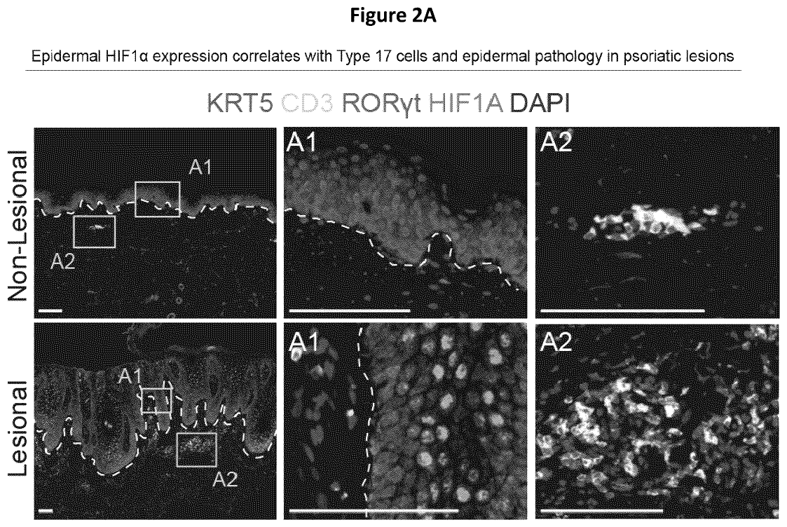

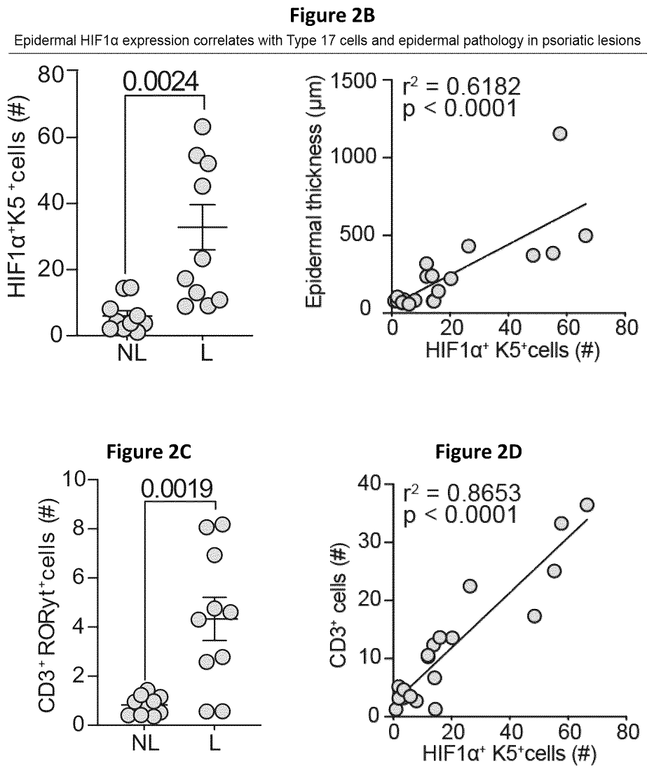

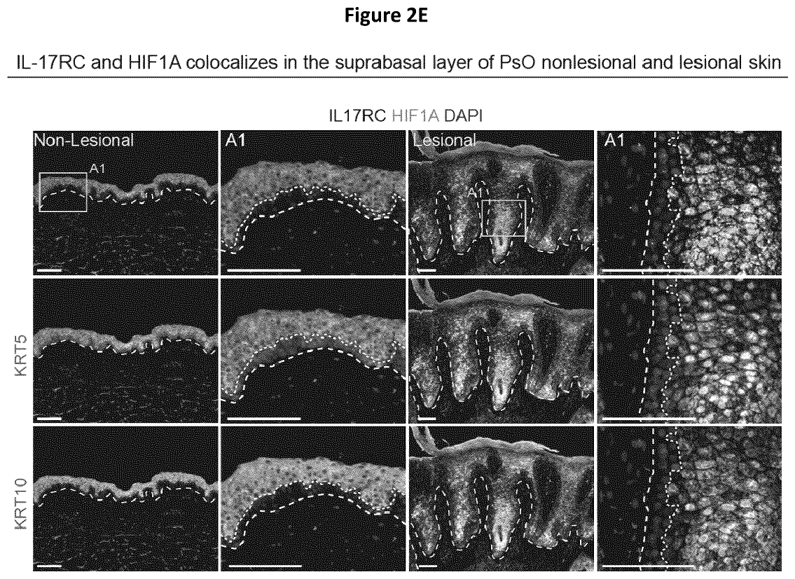

Given the well-established role of Type 17 immunity in driving psoriatic pathology, next the relationship between HIF1α activation and Type 17 cells was evaluated in a cohort of matched lesional and non-lesional samples from PsO patients. Immunofluorescence analysis revealed a positive correlation between epidermal HIF1α expression and epidermal pathology as measured by thickness of the Keratin (KRT) 5 layer (r²=0.6182) (FIGS. 2A-2B and 7D) and also with CD3⁺ cellular infiltrates (r²=0.8653) (FIGS. 2A, 2C-2D, and 7E).

The epithelium is comprised of functionally heterogeneous cell states consisting of basal stem and suprabasal differentiated cells. The relative contribution of these cell types in sensing and responding to inflammatory mediators in skin disease is unclear. The collected omics data clearly pointed to the suprabasal (differentiated) epithelium as a dominant target of inflammation in PsO. Accordingly, in human PsO lesions, HIF1α was exclusively activated (nuclear) in the Keratin 5⁺ Keratin 10⁺ double positive suprabasal layer but not Keratin 5⁺ single positive basal epithelium (FIG. 2E). This same pattern of activation was evident in AD and HS suprabasal epithelium (FIG. 7F), suggesting that HIF1α marks a common pathological state across a number of epithelial inflammatory diseases.

To further probe whether HIF1α activation may be uniquely marking IL-17A responsive epithelial populations, next the correlation of HIF1α activation with expression of IL-17 Receptor C (RC), a common receptor for IL-17A and F was evaluated (McGeachy et al., 2019). Surprisingly, in both healthy and PsO lesional human skin, IL-17RC was expressed in Keratin 10⁺ suprabasal epithelium, but not in the Keratin 5⁺ basal progenitor population (FIG. 2E). Notably, suprabasal epithelium in PsO lesions had a striking pattern of HIF1α and IL-17RC co-expression (100% concordance) (FIG. 7G), supporting the notion that epithelial HIF1α activation is a barometer of IL-17 signaling and downstream pathology.

### Example 3. Anti-IL-17A or Anti-TNFα Therapy-Responsiveness is Associated with Epidermal HIF1α Loss

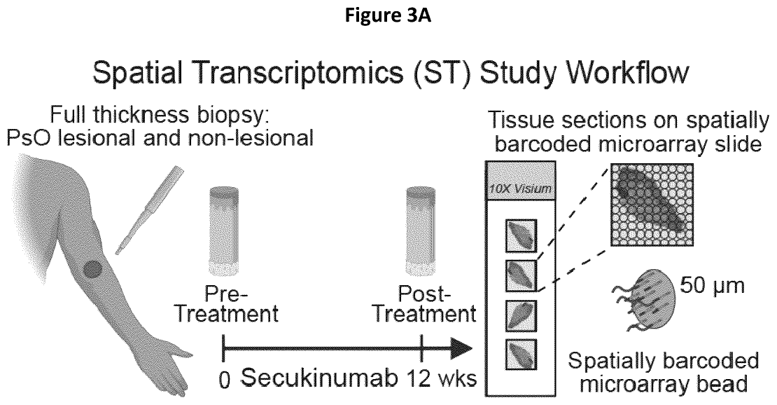

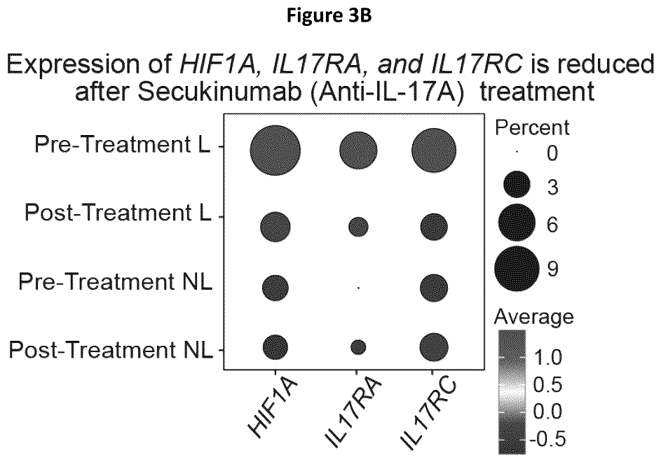

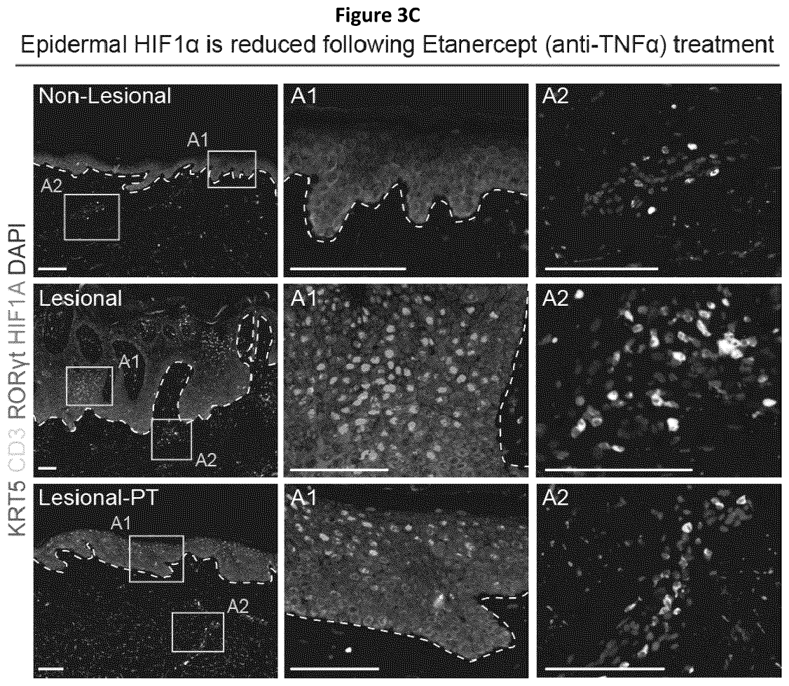

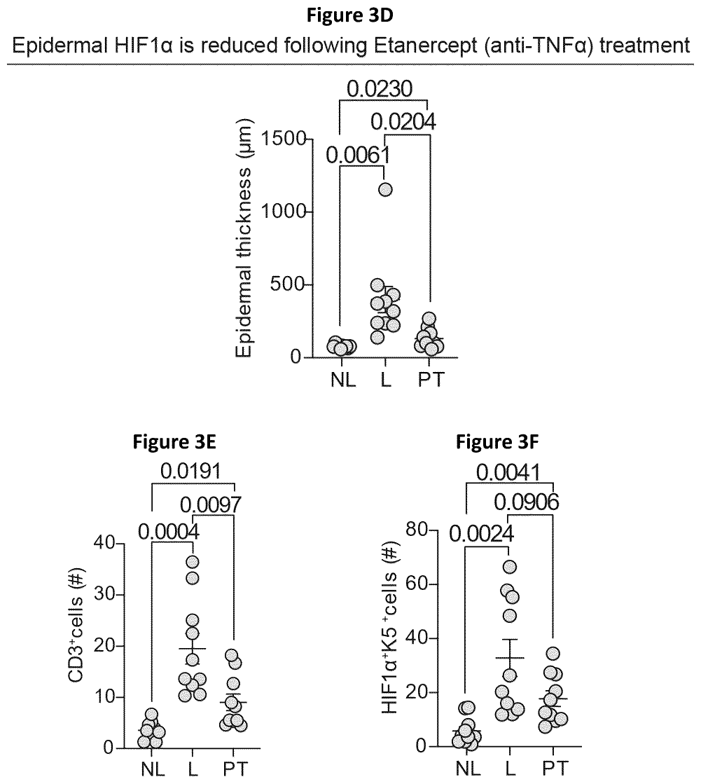

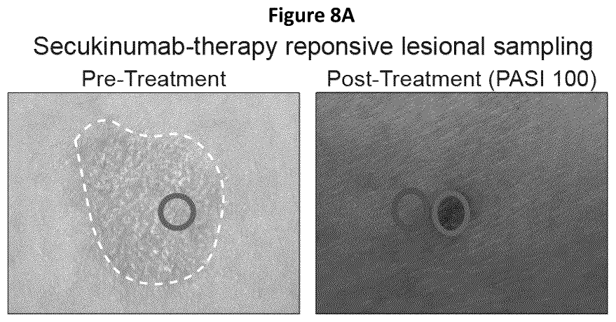

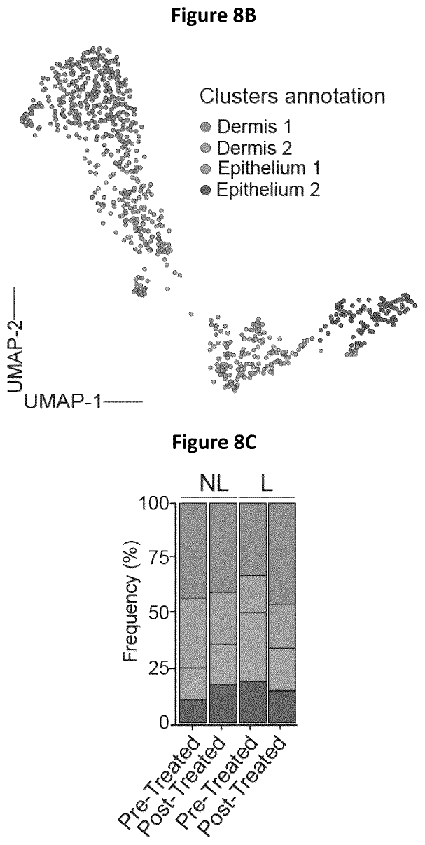

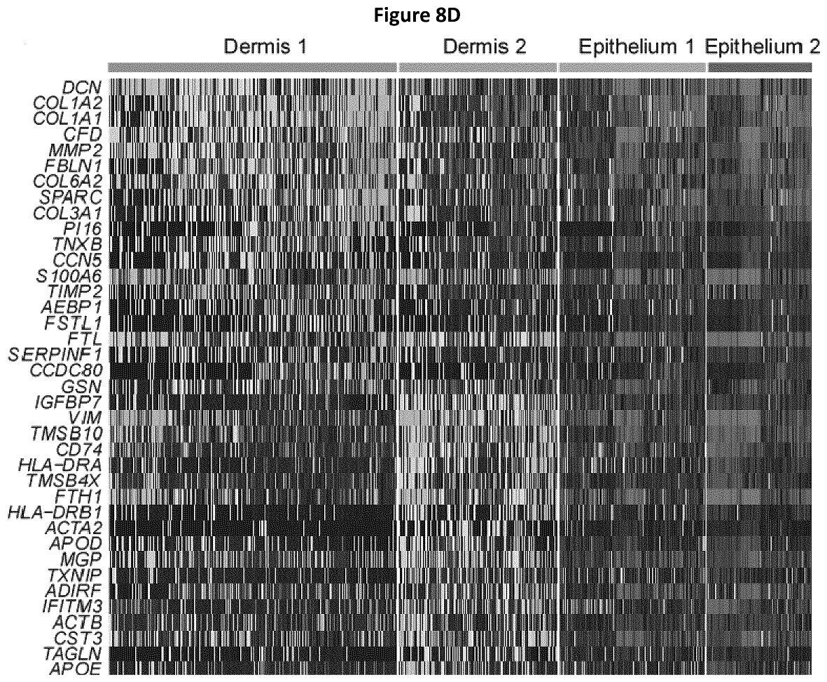

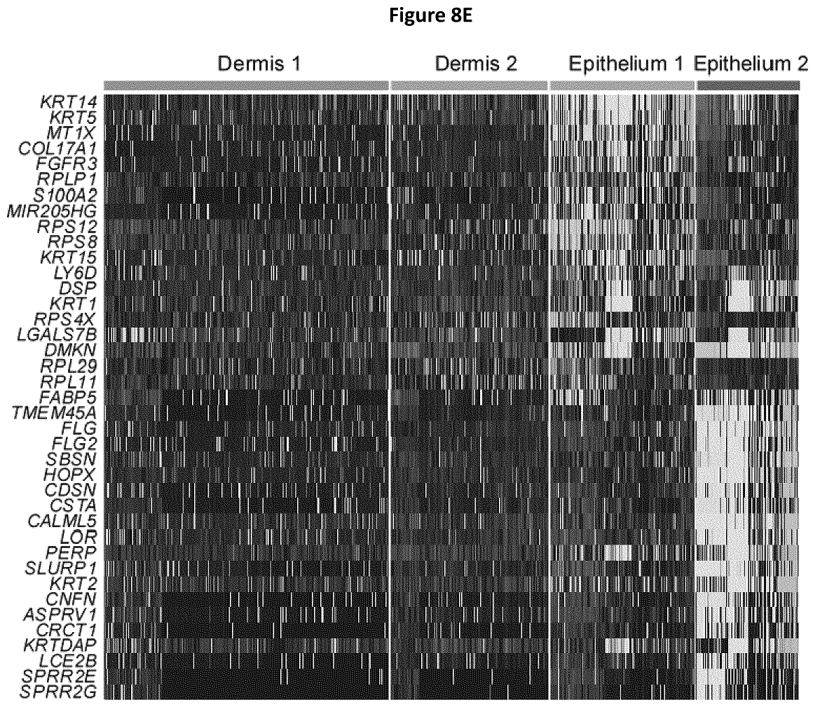

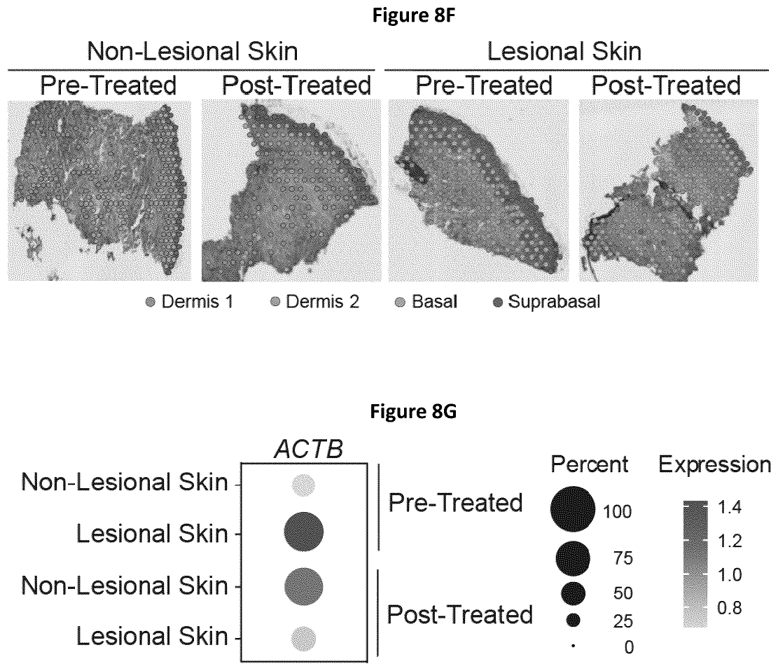

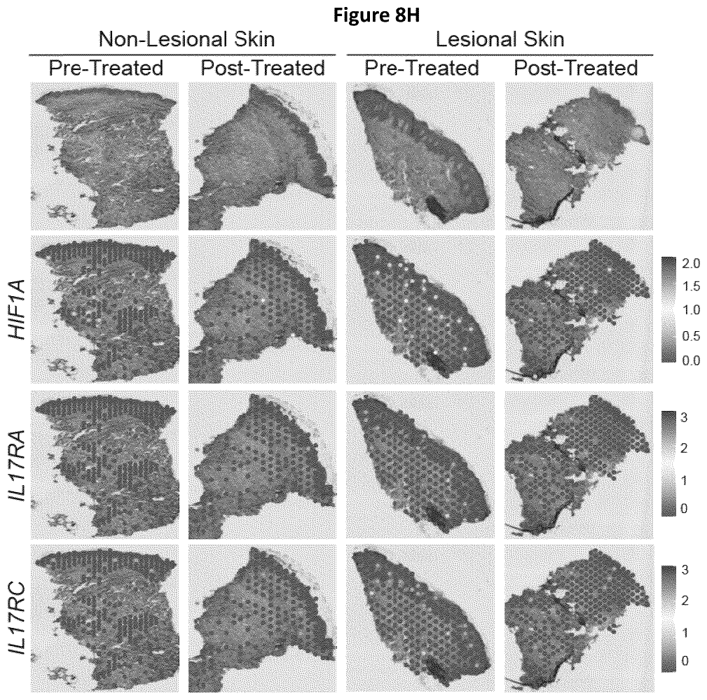

IL-17A blocking therapies are lauded for their clinical success, often achieving complete remission of skin plaques within 6 months of treatment (Ghoreschi et al., 2021). This remarkable clinical response provided a unique opportunity to examine if HIF1α levels normalize with successful therapy. Therefore, pre- and post-treatment biopsies were obtained from a secukinumab (anti-IL-17A) treated PsO patient who achieved complete clinical remission (Psoriatic Area and Severity Score (PASI) 100, or 100% psoriasis plaque clearance) (FIG. 3A and Table 1). Pre- and 12-weeks post-therapy biopsies from both lesional and non-lesional skin were taken <0.5 cm apart from the same patient, ensuring that post-treatment biopsies were taken from resolved lesions and not adjacent uninvolved skin (FIG. 8A).

TABLE 1
Clinical metadata from pre- and post- secukinumab-treated patient.

| ST ID | ST-15 L | ST-15 NL | ST-15 AL | ST-15 ANL |
|---|---|---|---|---|
| Age | 33 | 33 | 33 | 33 |
| Sex | F | F | F | F |
| Race/Ethnicity | White | White | White | White |
| Site | Left lateral | Right lateral | Left lateral | Right lateral |
| | thigh | thigh | thigh | thigh |
| Date biopsied | Sep. 25, 2020 | Sep. 25, 2020 | Jan. 8, 2021 | Jan. 8, 2021 |
| Diagnosis | Psoriatic | PsA | PsA | PsA |
| | arthritis (PsA) | | | |
| Body surface area (BSA) | 8 | 8 | 0 | 0 |
| with psoriasis (%) | | | | |
| PASI | 5 | 5 | 0 | 0 |
| Photo-type | 2 | 2 | 2 | 2 |
| Swollen joint count (SJC) | 11 | 11 | 0 | 0 |
| Tender joint count (TJC) | 12 | 12 | 0 | 0 |
| Dactylitis | 4 | 4 | 0 | 0 |
| Enthesitis | 1 | 1 | 0 | 0 |
| Deformity | 0 | 0 | 0 | 0 |
| Phys GA (global) | 5 | 5 | 1 | 1 |
| Meds at the time of biopsy | None | None | Secukinumab | Secukinumab |

Combined ST analysis of matched pre- and post-treatment samples from the same patient revealed 4 distinct spatial regions, of which the basal epidermal cluster was enriched in lesional pre-treatment skin (FIGS. 8B-8G). When compared to contralateral non-lesional skin, the PsO lesion was enriched for HIF1A, IL17RA, and IL17RC (FIGS. 3B and 8H). Notably, following therapy, expression of HIF1A, IL17RA, and IL17RC in resolved skin (post-treatment L) were comparable to that of non-lesional skin (FIGS. 3B and 8F-8G). As a complementary analysis, HIF1α protein expression was measured in a cohort of 10 patients treated with etanercept (anti-TNFα) that achieved response (PASI 75 or greater) at 12 weeks (Table 2) (Stinco et al., 2019). Etanercept-responsive patients had reduced epidermal hyperplasia (FIGS. 3C and D). Consistent with diminished HIF1A expression post-secukinumab, expression of epidermal HIF1α was also reduced following etanercept treatment in responsive patients (FIGS. 3C, 3F, and 8I). Etanercept has been reported to diminish Th17-mediated release of IL-17 in responsive patients, suggesting that diminished HIF1α in etanercept may also be linked to reduction of IL-17 following TNFα blockade. Indeed, etanercept responsive patients also exhibited a reduction in cutaneous Type 17 cells (FIGS. 3C and E) (Zaba et al., 2007). Collectively, these analyses make a compelling argument for exuberant IL-17A signaling and subsequent HIF1α induction in fueling a pathological epithelial state that is resolved following successful biologic therapy, irrespective of cytokine target.

TABLE 2
Clinical metrics of PsO patients over course
of etanercept (anti-TNFα) therapy.

| | | | | Static |
|---|---|---|---|---|
| | | | | Physician's |
| | | | | Global |
| | | | | Assessment |
| Patient | Week | PASI | BSA | (SPGA) |
| Patient 28 | Week 0 | 11 | 9 | 3 |
| | Week 2 | 9.5 | 9 | 3 |
| | Week 6 | 7.4 | 8 | 3 |
| | Week 12 | 4.5 | 6 | 2 |
| Patient 29 | Week 0 | 11.6 | 28 | 3 |
| | Week 2 | 3.9 | 22 | 2 |
| | Week 6 | 2.7 | 4 | 1 |
| | Week 12 | 2.7 | 3 | 1 |
| Patient 30 | Week 0 | 10.3 | 20 | 3 |
| | Week 2 | 8.1 | 15 | 2 |
| | Week 6 | 4.8 | 15 | 2 |
| | Week 12 | 2.4 | 10 | 1 |
| Patient 31 | Week 0 | 10.1 | 13 | 3 |
| | Week 2 | 9.4 | 11 | 3 |
| | Week 6 | 8.4 | 11 | 3 |
| | Week 12 | 11.6 | 15 | 3 |
| Patient 32 | Week 0 | 16.1 | 13 | 4 |
| | Week 2 | 14.3 | 12 | 4 |
| | Week 6 | 4.4 | 8 | 2 |
| | Week 12 | 3.2 | 3 | 1 |
| Patient 33 | Week 0 | 34.4 | 80 | 4 |
| | Week 2 | 31.5 | 76 | 3 |
| | Week 6 | 15 | 39 | 2 |
| | Week 12 | 12 | 27 | 2 |
| Patient 35 | Week 0 | 19.4 | 31 | 3 |
| | Week 2 | 17.4 | 29 | 3 |
| | Week 6 | 6.6 | 25 | 2 |
| | Week 12 | 0 | 1 | 2 |
| Patient 38 | Week 0 | 15.8 | 25 | 3 |
| | Week 2 | 18.8 | 15 | 4 |
| | Week 6 | 12.3 | 13 | 2 |
| | Week 12 | 2 | 7 | 2 |
| Patient 39 | Week 0 | 15 | 20 | 3 |
| | Week 2 | 10.7 | 19 | 3 |
| | Week 6 | 8.2 | 18 | 3 |
| | Week 12 | 8.4 | 18 | 3 |
| Patient 40 | Week 0 | 12 | 20 | 3 |
| | Week 2 | 10.8 | 20 | 3 |
| | Week 6 | 4.5 | 16 | 2 |
| | Week 12 | 4.5 | 8 | 1 |

### Example 4, Ex Vivo HIF1α Inhibition of PsO Lesions Modulates Metabolism and has a Greater Effect than the Topical Standard of Care

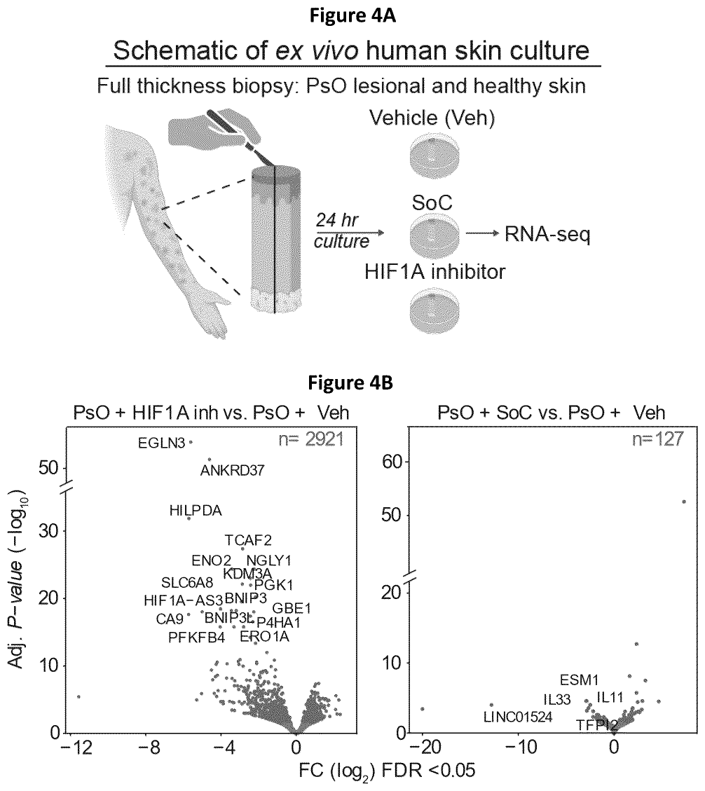

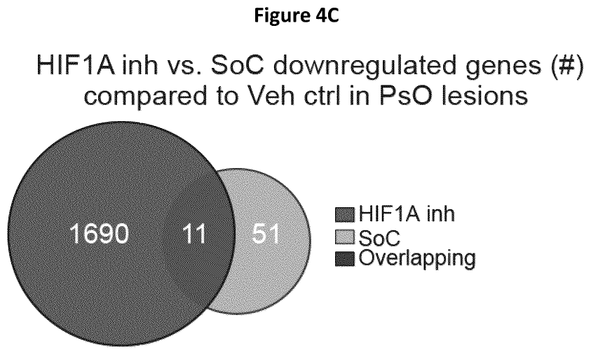

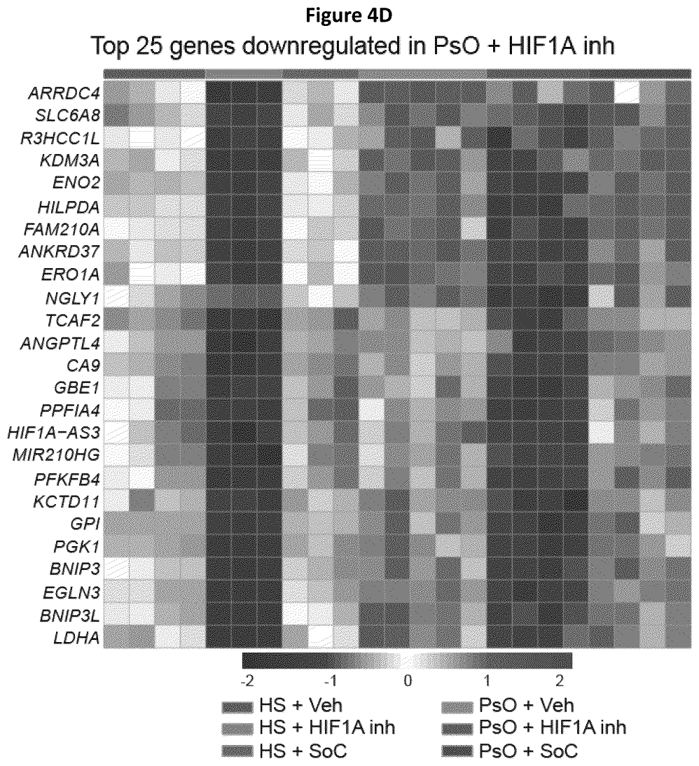

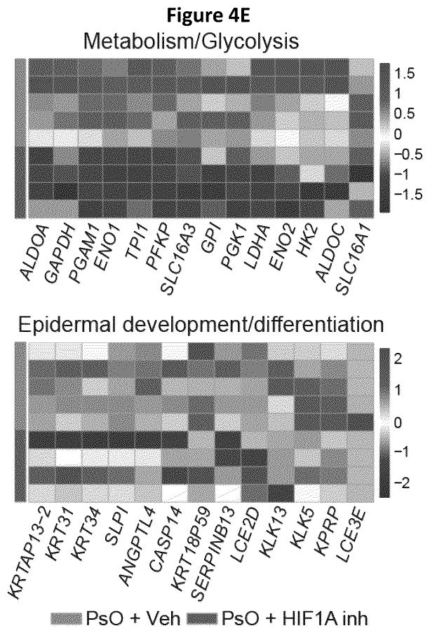

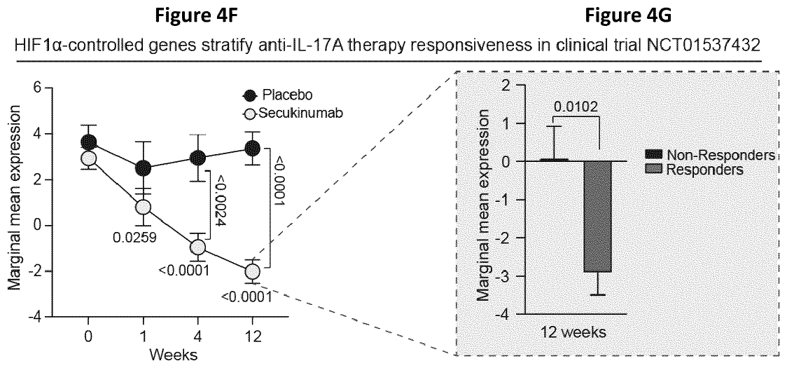

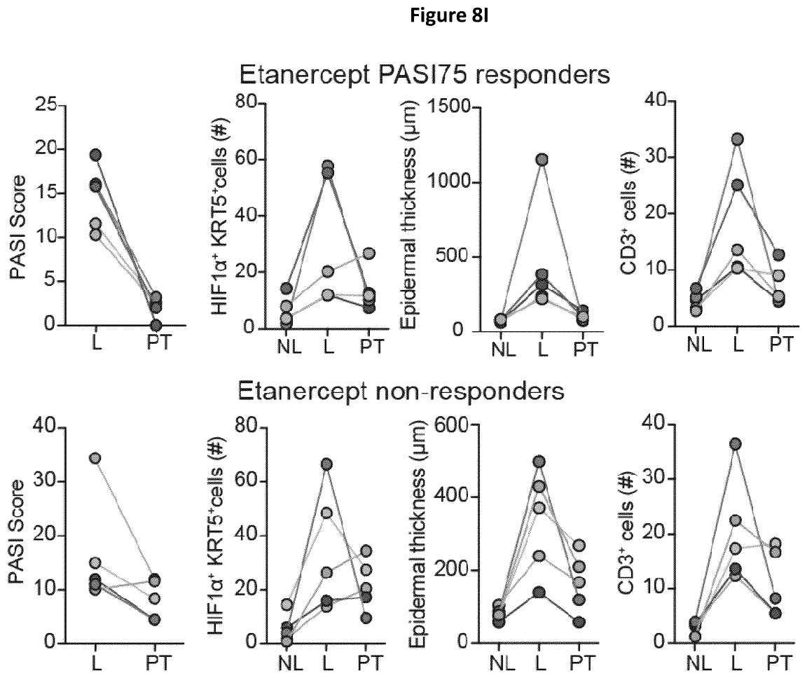

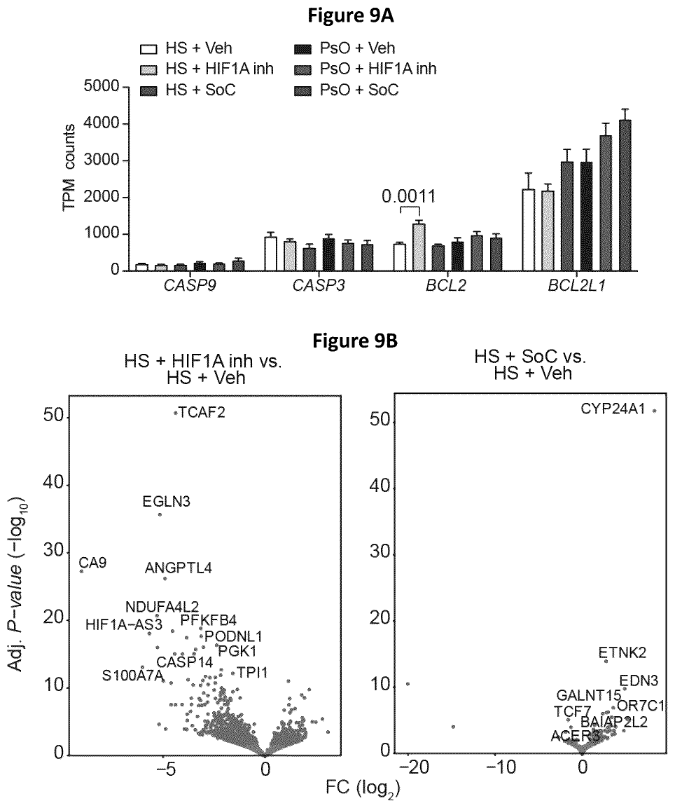

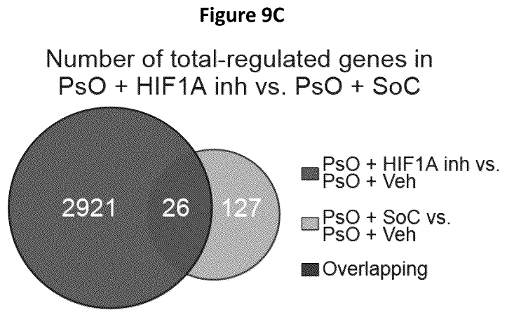

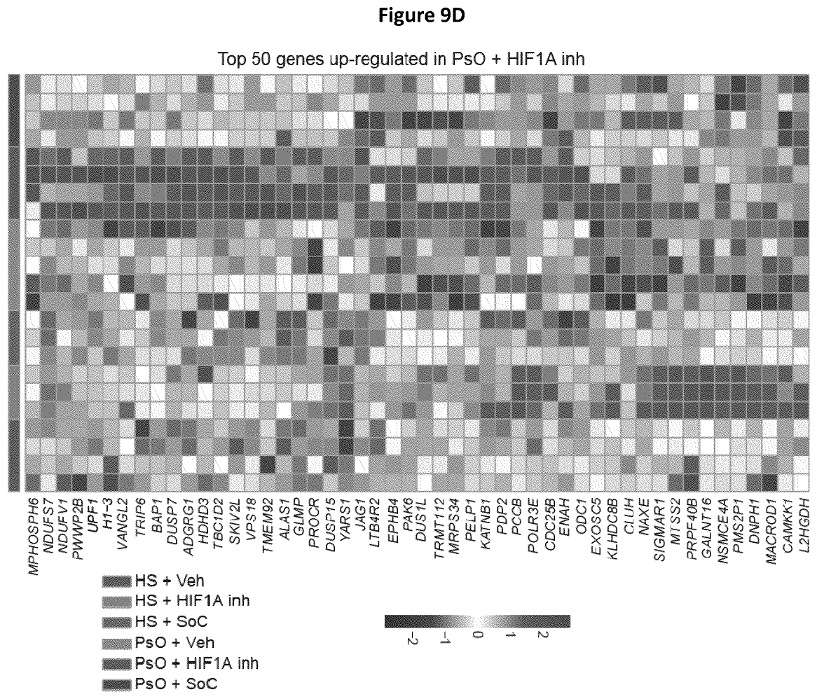

The notable loss of epidermal HIF1α in biologic therapy-responsive patients raised the tantalizing possibility that this factor may be a key orchestrator of disease. Therefore, an ex vivo “trial in a dish” system was devised to test the effects of HIF1α inhibition on PsO lesional human skin. Lesional biopsies were obtained from 5 PsO patients that were either therapy-naïve or non-responsive to biologic therapy (Table 3). Each patient biopsy was divided prior to treatment with either vehicle control, a HIF1α inhibitor (BAY 87-2243), or topical standard of care (SoC, calcipotriene and betamethasone dipropionate) for 24 hours in culture (FIG. 4A) (Ellinghaus et al., 2013; Rogalski, 2015). Surgical foreskin discards were used as healthy control skin and subject to the same treatment regimens. All samples underwent bulk transcriptomics analysis (RNA-seq) to determine global changes to tissues induced by HIF1α inhibition or SoC. It was confirmed that the treatment conditions did not alter apoptosis genes expression in healthy and PsO cultures, indicating that changes in gene expression in downstream analysis were not a result of cell death following treatment (FIG. 9A).

TABLE 3
Clinical metadata from biopsied PsO patients for ex vivo culture.

| ID | PREP-2001-V1 | PREP-2006 | Micra-1009 | Micra-1010 | Micra-1012 |
|---|---|---|---|---|---|
| Age | 68 | 35 | 26 | 52 | 36 |
| Sex | M | F | M | M | M |
| Race/ | White | Hispanic | Hispanic | Caucasian | Caucasian |
| Ethnicity | | | | non-Hispanic | non-Hispanic |
| Site | Left flank | Left distal leg × | Left elbow × | Right leg | Right leg |
| | | 2 lesional sites | 2 lesional sites | | |
| Diagnosis | PsO | PsO | PsA | PsA | PsA |
| Clinical site | Center for | CMC | CMC | CMC | CMC |
| | Musculoskeletal | | | | |
| | Care (CMC) | | | | |
| Biopsy size | 4 | 4 × 2 sites | 4 × 2 sites | 4 × 1 site | 4 × 2 sites |
| BSA (%) | 50 | 2.5 | 1 | 5 | 6 |
| PASI | 26 | 2.8 | 1.9 | 3.5 | 8.2 |
| Photo-type | 2 | 4 | 4 | 2 | 2 |
| Meds at the | None | None | Certolizumab | Adalimumab, | Izekizumab |
| time of biopsy | | | | ustekinumab | |
| Biologic | Never | Skyrizi | Adalimumab | Secukinumab, | Ustekinumab |
| history | | | | golimumab | |

Differential gene expression analysis between vehicle and treated lesional biopsies revealed a pronounced effect of HIF1α inhibition that was notably greater than SoC in both lesional and healthy skin conditions (FIGS. 4B and 9B-9C). Compared to vehicle control, HIF1α inhibition of PsO lesional skin resulted in 2921 differentially expressed genes (DEGs), while SoC resulted in a much smaller response with only 127 DEGS (FDR<0.05) (FIGS. 4B-4C). Genes associated with glycolytic metabolism (ENO2, PGK, GPI, PFKB4), STAT3 activation (KDM3A, ANGPTL4), and epithelial development/differentiation (KRT31, KRT34, CASP14, KLK5) were strikingly downregulated following BAY 87-2243 treatment (FIGS. 4D-4E).

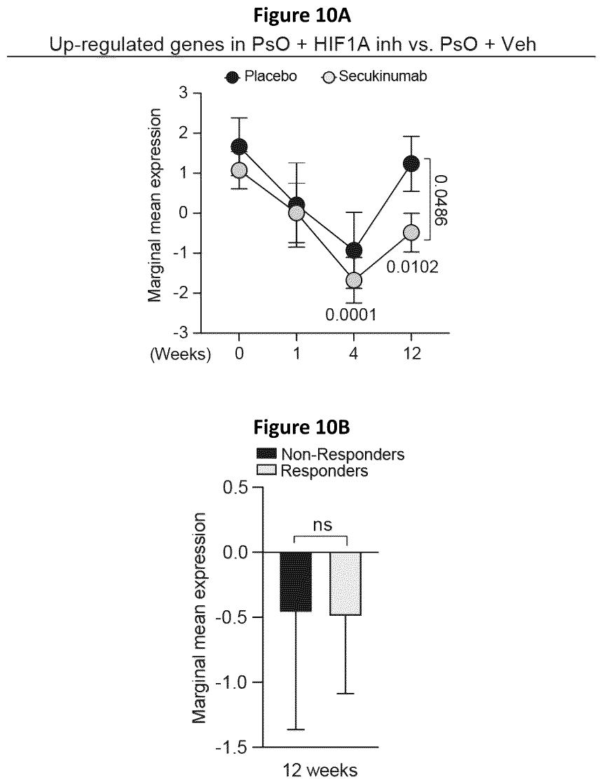

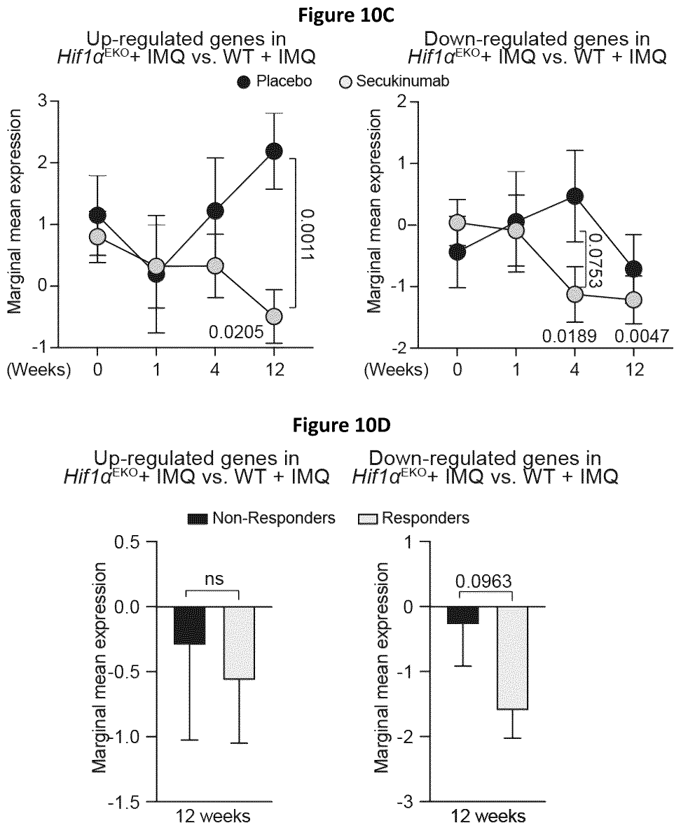

The dramatic transcriptional response following BAY 87-2243 treatment prompted investigation of the role of HIF1α-modulated genes on PsO disease pathogenesis. Toward this end, HIF1α-modulated genes were projected onto a longitudinal analysis of the transcriptional response from 33 patients following either placebo (9 patients) or anti-IL-17A blocking (secukinumab) therapy (24 patients) (NCT01537432) (Krueger et al., 2019). Genes upregulated following HIF1α inhibition, relative to vehicle control, did not distinguish between secukinumab responders or non-responders (FIGS. 9D and 10A-10B). By contrast, the downregulated gene signatures significantly distinguished the anti-IL-17A treatment group from placebo (FIG. 4F). Intriguingly, changes in these genes were observed at 1-week post therapy, even before clinical response, and were most pronounced at 12 weeks when lesions resolved. Furthermore, at 12 weeks, the transcriptional signatures of therapy-responsive (PASI 75) versus non-responsive patients significantly stratified by the HIF1α-inhibitor downregulated gene signature, underscoring the importance of this program in driving skin pathology downstream of IL-17A (FIG. 4G).

### Example 5. Epidermal HIF1α is Necessary for Psoriatic Pathology in Murine Models

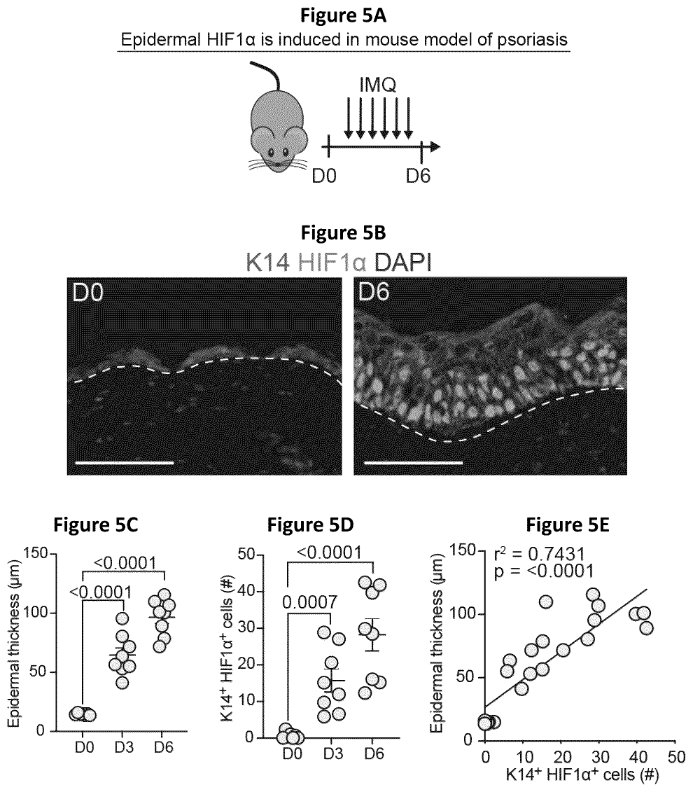

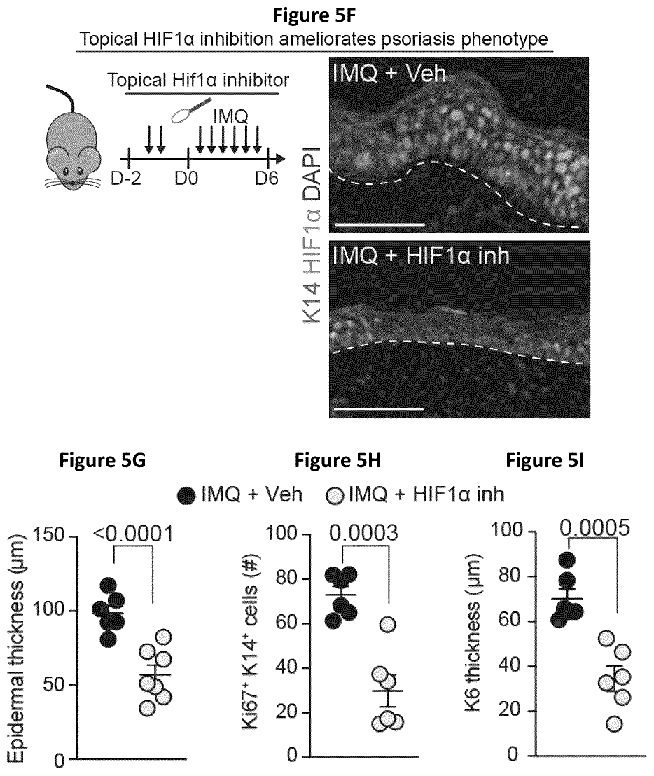

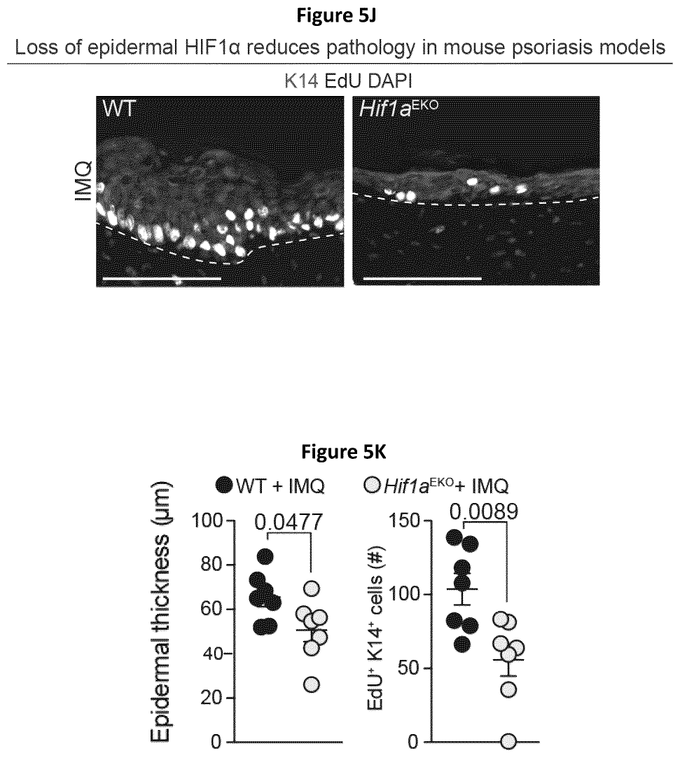

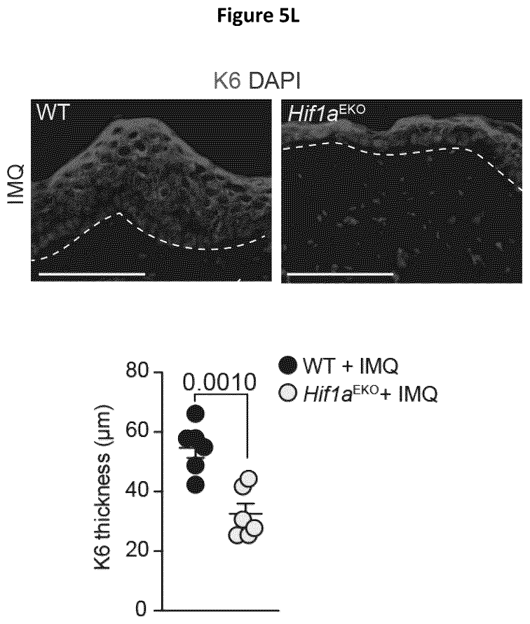

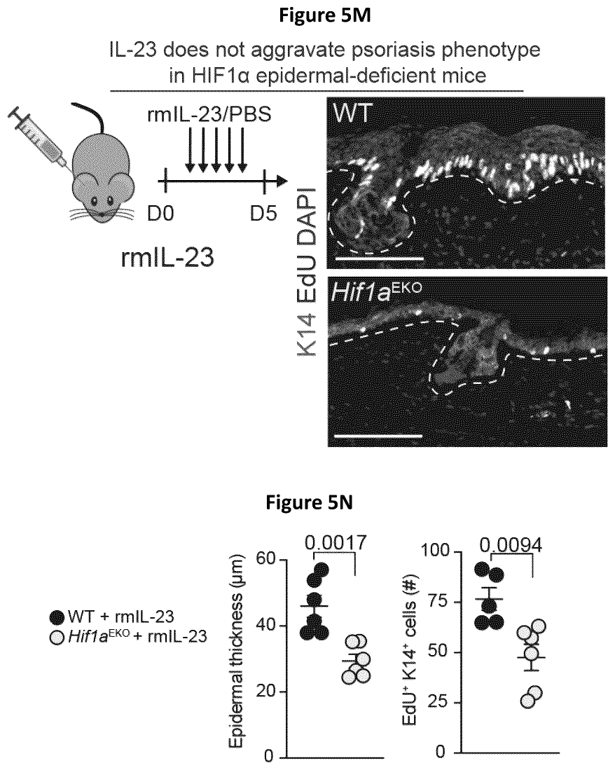

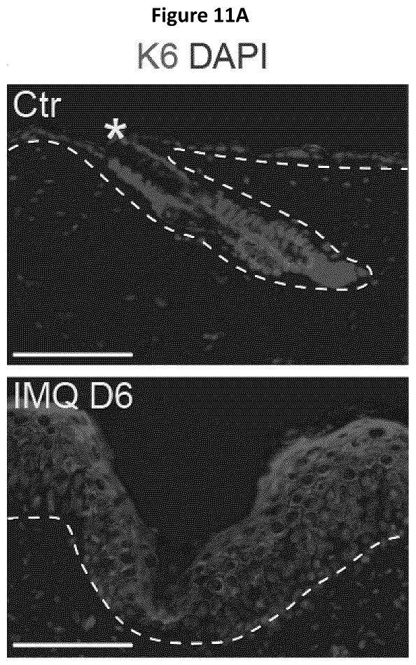

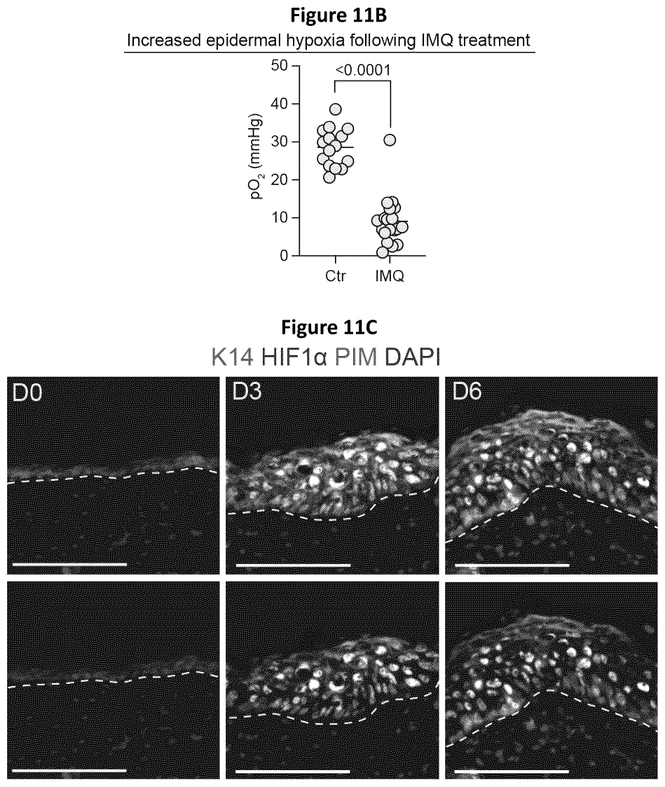

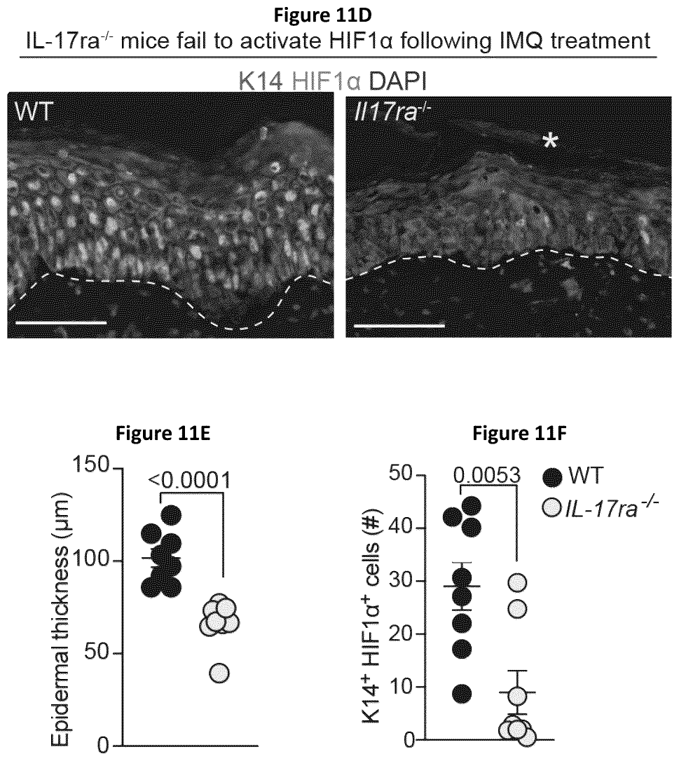

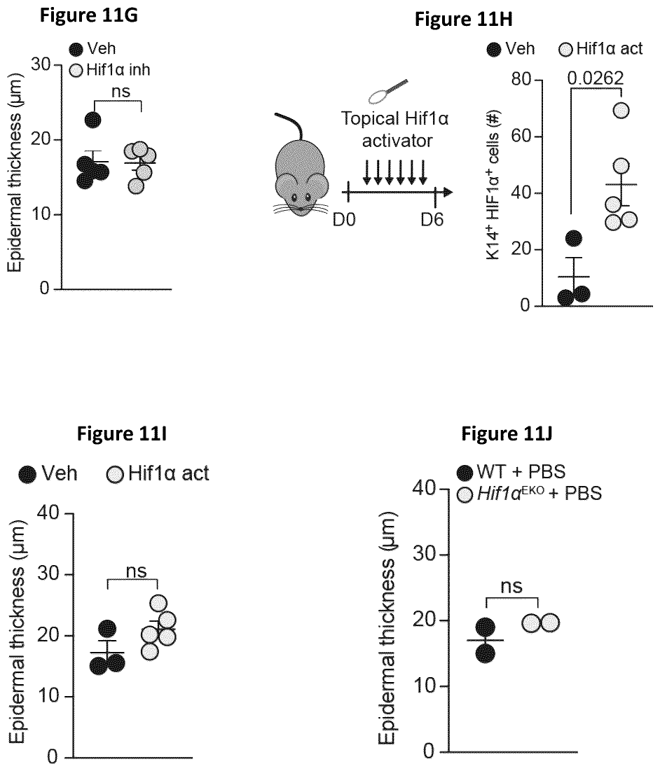

The human studies thus far pointed to HIF1α-controlled metabolic remodeling as a key pathological effect of exuberant IL-17A signaling. To probe a causal role for epithelial HIF1α activation in driving disease pathology, a well-established mouse model of psoriatic-like inflammation induced by topical application of a TLR-7 agonist, imiquimod (IMQ) was used (van der Fits et al., 2009). The dysfunctional epithelial state identified in the human samples was marked by Keratin 6 expression in IMQ-treated mice (FIG. 11A). Importantly, IMQ-treated epidermis robustly upregulated HIF1α at the onset of pathology (day 3 of treatment), which was heightened at the peak of disease (day 6) (FIGS. 5A-5E). However, in contrast to human PsO skin, in which HIF1α expression was restricted to suprabasal epidermis, murine epidermis expressed HIF1α in both basal and differentiated cell compartments, indicating some species-specific differences in the pattern of HIF1α activation (FIG. 5B).

Next tissue oxygen levels were measured with a fiberoptic probe and a hypoxic microenvironment in IMQ-treated skin was uncovered (FIG. 11B). However, immunofluorescence imaging with a chemical probe, pimonidazole (PIM), revealed that HIF1α expression was not exclusive to PIM⁺ areas of hypoxia (FIG. 11C). This was particularly evident at the peak of inflammation when PIM signal was enriched in only the uppermost epidermal layers while HIF1α was robustly activated throughout the epidermis. Therefore, it was examined whether IL-17A signaling is a key trigger of HIF1α in PsO, consistent with previous findings in epithelial repair (Konieczny et al., 2022). Indeed, mice globally deficient in IL-17RA were unable to activate HIF1α with a corresponding decrease in epithelial pathology (FIGS. 11D-11F).

The aforementioned findings suggest that epidermal HIF1α activation downstream of IL-17A is a central amplifier of epidermal dysfunction in PsO. Therefore, it was tested whether topical HIF1α inhibition with BAY 87-2243 could alter IMQ-induced inflammatory pathology in vivo (FIG. 5F) (Ellinghaus et al., 2013). Remarkably, HIF1α inhibition protected mice from epidermal pathology, as BAY 87-2243-treated mice had significantly reduced epidermal thickness, proliferation, and inflammatory Keratin 6 expression (FIGS. 5G-5I). Conversely, vehicle control-treated epidermal thickness was not altered by BAY 87-2243 treatment (FIG. 11G).

To examine if HIF1α is sufficient to promote epithelial pathology in the absence of inflammation, a HIF1α activator, FG-4592, was used (Wu et al., 2016). Topical FG-4592 application effectively heightened epidermal HIF1α levels (FIG. 11H). Yet, there was no observation of significant differences in epidermal thickness or any overt signs of skin inflammation (FIG. 11I), indicating that in the absence of an inflammatory milieu, HIF1α alone does potentiate disease.

Next, to assess the necessity of epidermal HIF1α in fueling disease, an epithelial-specific HIF1α knockout mouse (Keratin (K) 14Cre; Hif1a^{fl/fl}, herein referred to as Hif1a^{EKO}) was employed. Without inflammation, loss of HIF1α did not alter epidermal thickness (FIG. 11J), consistent with the findings that modulating HIF1α activity in healthy skin does not in and of itself lead to inflammation. Remarkably, following IMQ, Hif1a^{EKO} had significant decrease in epidermal thickness, hyperproliferation (as measured by EdU incorporation), and epidermal dysfunction (Keratin 6 expression) (FIGS. 5J-5L). These findings were corroborated in an IL-23-induced model of PsO (FIG. 5M) (Chan et al., 2006). When compared to wildtype controls, Hif1a^{EKO} mice had a marked reduction in epidermal thickness and proliferation following intradermal recombinant murine (rm) IL-23 administration (FIG. 5N). Collectively, these data support the notion that epidermal HIF1α is a key node involved in translating IL-17A signaling and pathologically remodeling epithelia.

### Example 6. HIF1α Controls PsO Pathology Via Epidermal Intrinsic and Microenvironmental Metabolic Modulation

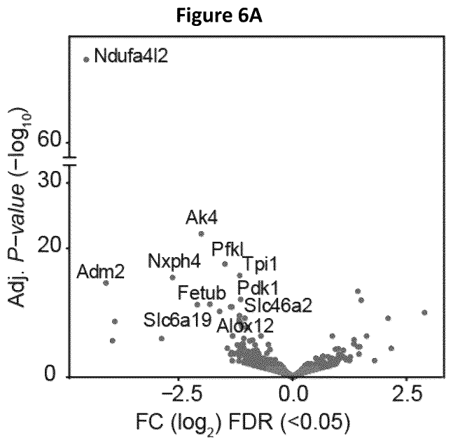

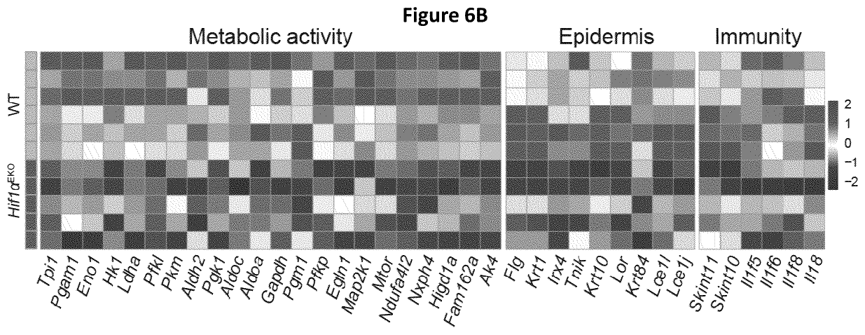

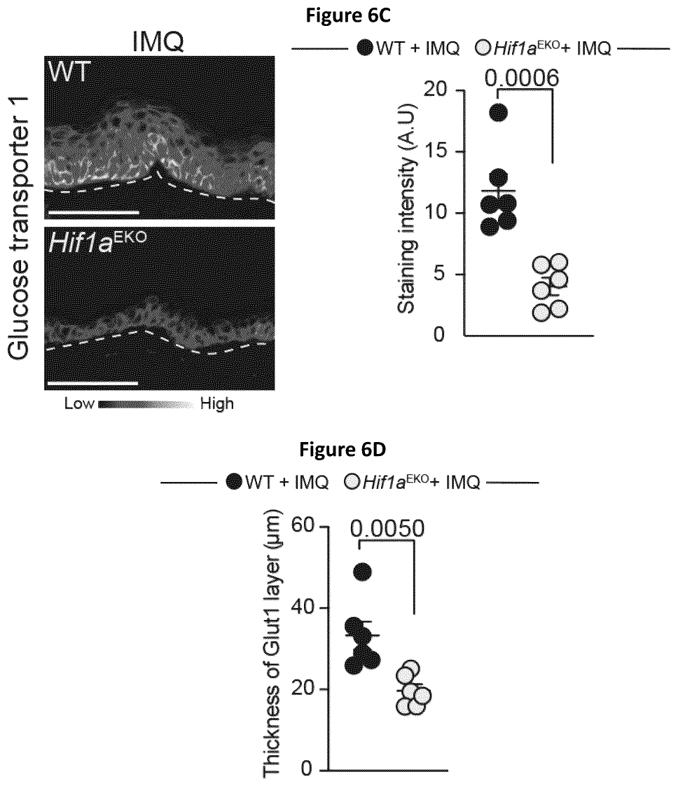

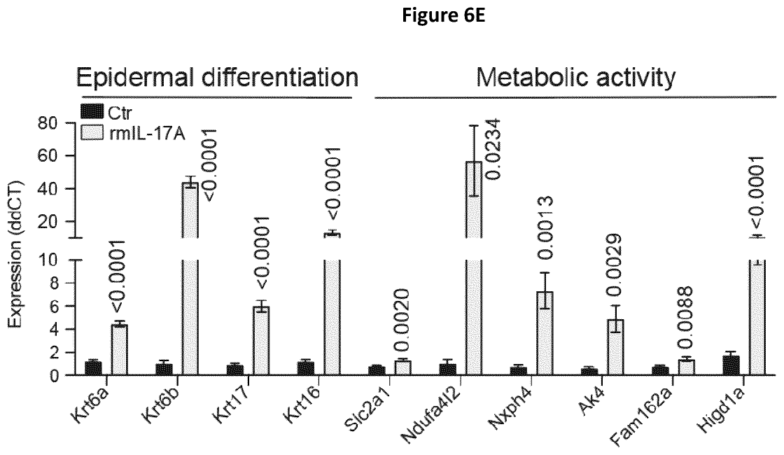

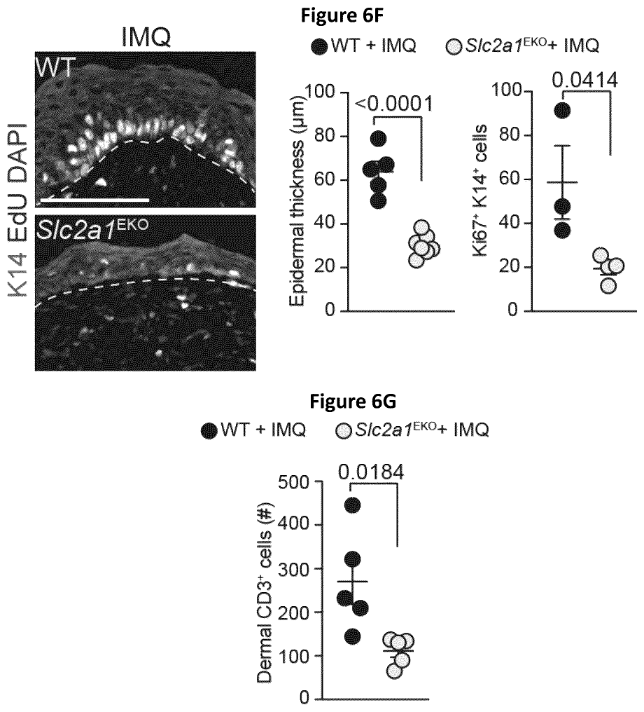

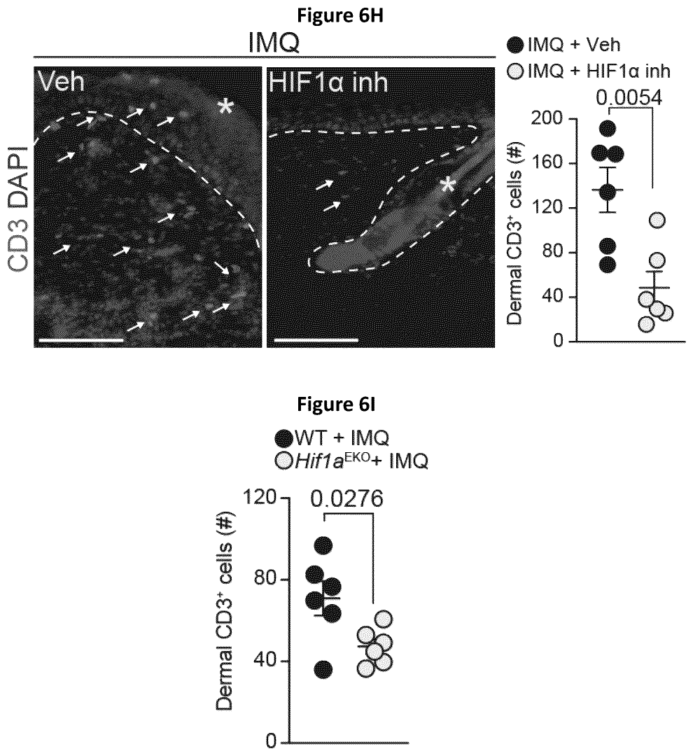

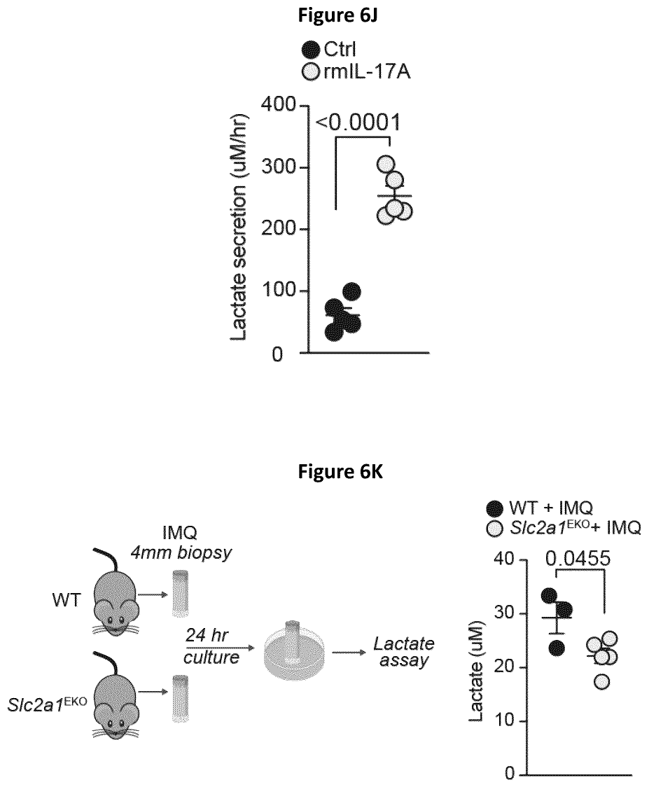

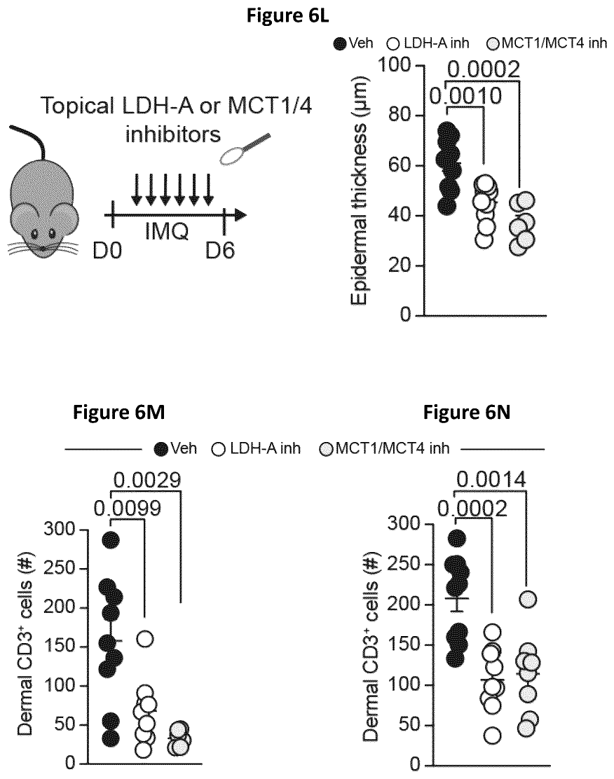

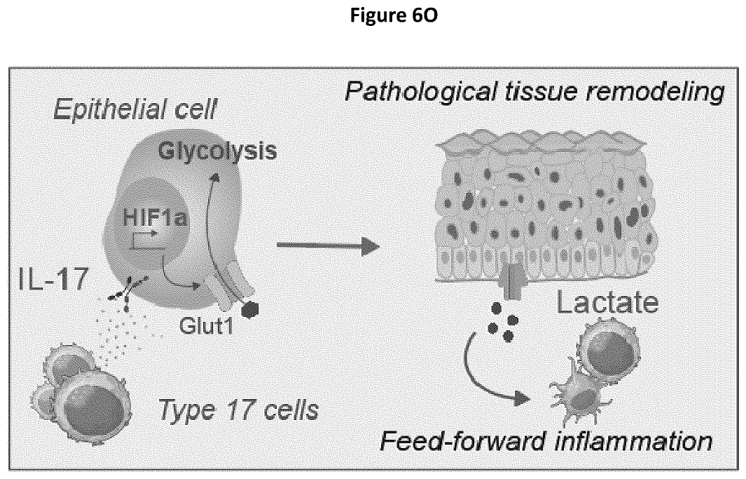

To delve deeper into molecular mechanism, next global gene expression of Hif1a^{EKO} and WT skin was evaluated following IMQ treatment using bulk tissue RNA-seq. Comparative bioinformatics revealed that Hif1a^{EKO} and WT skin had 387 DEGs (<0.5 FDR) (FIG. 6A). Similar to DEGs from human PsO lesions treated with HIF1α inhibitor (FIG. 4C), albeit to a lower extent, the DEGs from Hif1a^{EKO} and WT murine skin also separated secukinumab-treated patients from placebo controls (FIGS. 10C-10D). By examining the genes that were downregulated in Hifa^{EKO} relative to WT controls, reduced expression was observed for transcripts associated with glycolysis (Eno1, HK1, Ldha, Pfkp), mitochondrial function (Ndufa412, Nxph4, Higd1a, Fam162a), epidermal differentiation (Krt1, Krt10, Flg, Lor, Lceli), and immunity (Skint11, Skint10, Illf5, Illf6, Illf8, Il18) (FIG. 6B).

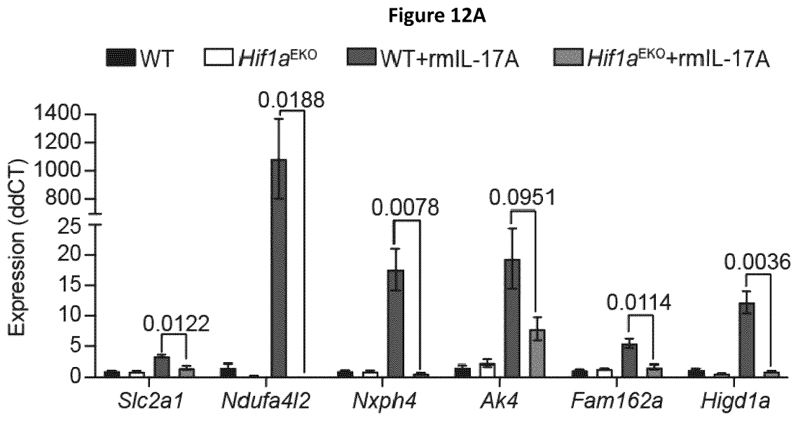

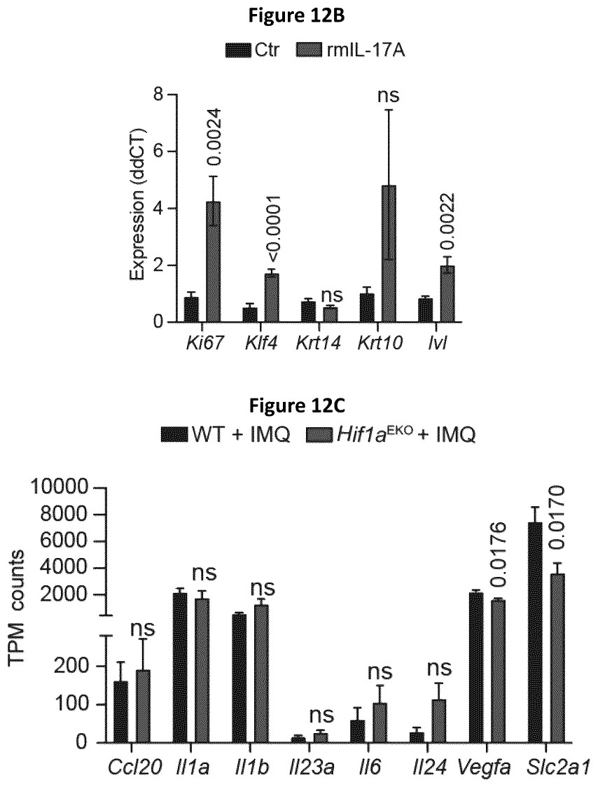

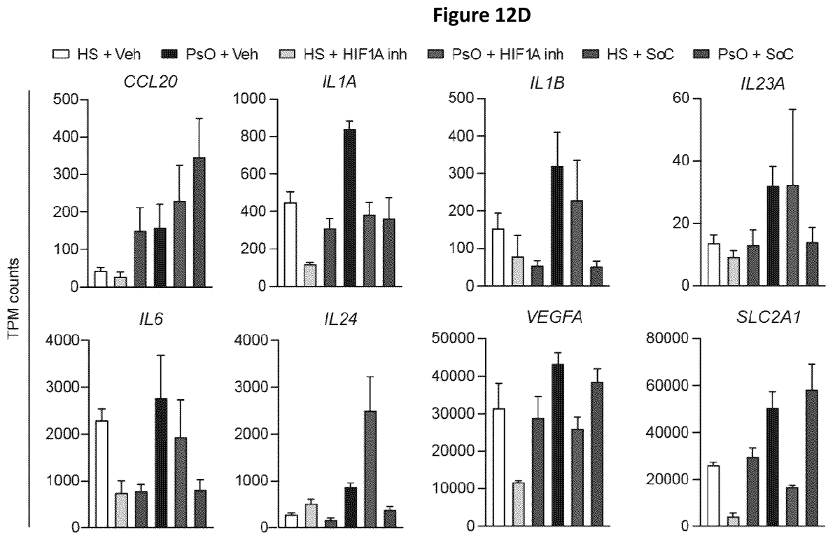

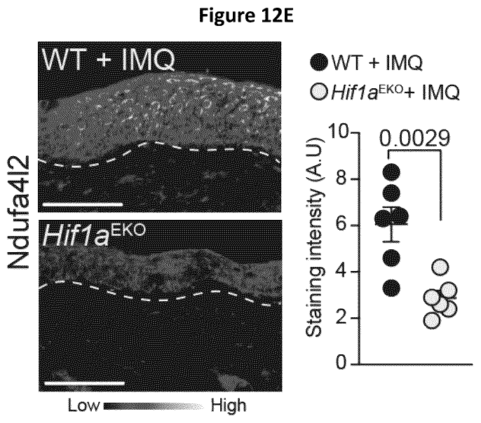

To functionally validate the HIF1α regulated-metabolic and differentiation defects in the metabolic program of PsO epithelia, first protein expression was examined of two key metabolism-associated target genes, glucose transporter 1 (Glut1, Slc2a1) and NDUFA4L2, a subunit of the ubiquinone enzyme, in vivo. Wildtype mice treated with IMQ had membrane-associated expression pattern of Glut1 in the lower layers of the epidermis and cytoplasmic expression of the mitochondrial complex 1 protein NDUFA4L2 in the upper epidermal layers (FIGS. 6C-6D and 12E). Hif1a^{EKO} animals, on the other hand, has significantly lower expression of Glut1 and NDUFA4L2 (FIGS. 6C-6D and 12E). Also was examined the response of inflammation in a primary organoid system made exclusively of murine skin epithelia without any accessory cells following IL-17A stimulation (FIG. 6E). It was found that rmIL-17A treatment in a primary epithelia organoid system recapitulated the epidermal dysfunction (Krt6a, Krt6b, Krt17) and glycolysis programs of PsO epidermis. This heightened expression of differentiation and metabolic genes was notably missing in Hif1a^{EKO} organoids (FIG. 12A-12B). These findings suggest that HIF1α-mediated epidermal intrinsic metabolic rewiring is at the crux of the PsO disease program.

To assess the epidermal intrinsic role of glycolytic metabolism, epidermal-specific Glut1-deficient animals (Keratin (K) 14Cre; Slc2a1^{fl/fl}, herein referred to as Slc2a1^{EKO}) were generated. Remarkably, Slc2a1^{EKO} mice were entirely protected from IMQ pathology and significantly reduced epidermal thickness and proliferation (FIG. 6F). These striking results raised the tantalizing possibility that modulating epithelial metabolism would be sufficient to curtail the immune response. Consistent with the feed forward epithelial-immune circuit model, topical HIF1α inhibition (BAY 87-2243), Hif1a^{EKO} mice, and Slc2a JEKO mice all had significantly lower numbers of dermal T cells compared to their respective controls (FIGS. 6G-6I). Thus, it was sought to determine the precise means by which such crosstalk with immunity may occur. Toward this end, the expression of factors important in driving type 17 immunity that drives PsO inflammation were evaluated. These include key chemokines and cytokines CCL20, IL-1, IL-6, and IL-23 that recruit and sustain Type 17 cell function in the skin (McGeachy et al., 2019). Surprisingly, and in contrast to canonical HIF targets (Vegfa, Slc2a1), Ccl20, Il1a, Il1b, 116, 1123a transcripts were not differentially expressed between Hif1a^{EKO} and WT skin (FIG. 12C). Nor were transcripts of factors proposed to be controlled by HIF1α in cultured epithelium following acute hypoxia, such as Il24 (FIG. 12C).

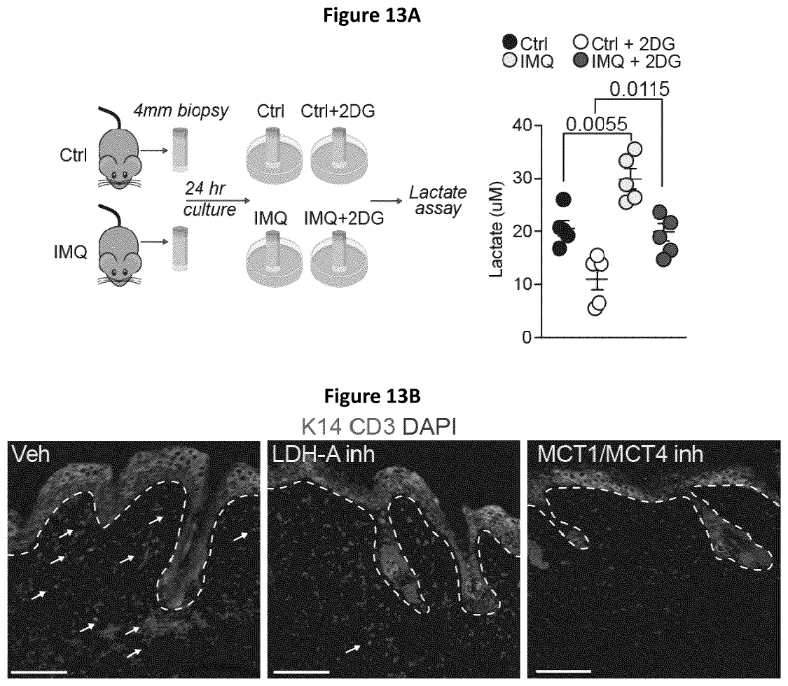

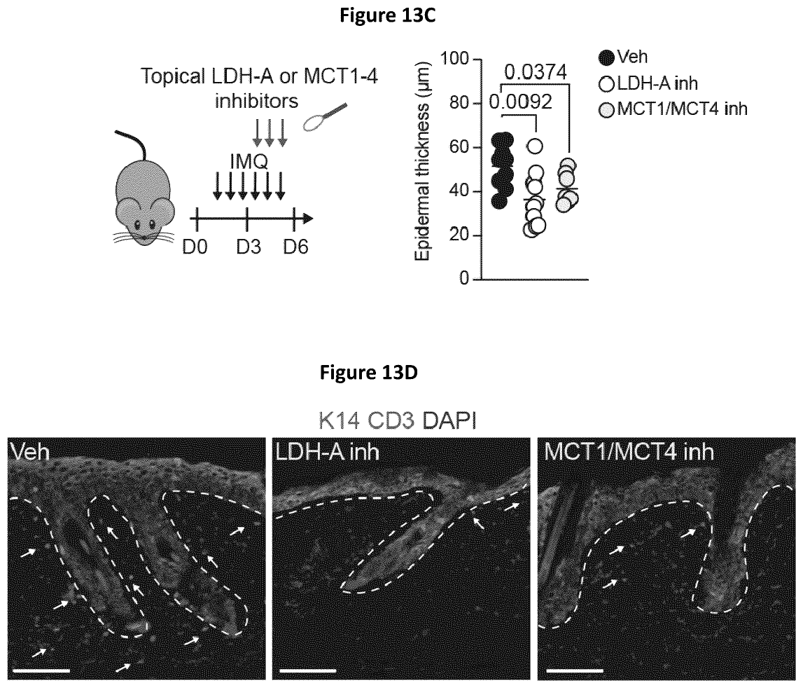

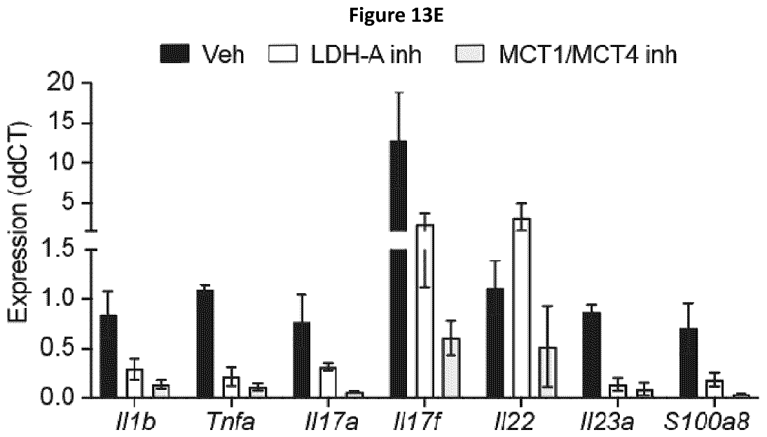

It was therefore hypothesized that a metabolite undetectable by the transcriptomics analysis could be mediating the pathological effects of epidermal HIF1α-induced glycolysis. Lactate, a terminal end product of glycolytic metabolism, was highly produced following IL-17A treatment of organoids compared to controls (FIG. 6J) (Konieczny et al., 2022; Rabinowitz and Enerback, 2020). In addition, the expression of both lactate transporters SLC16A1 (MCT1) and SLC16A3 (MCT4) was significantly reduced following HIF1α inhibition of human PsO, but not standard of care (FIG. 4E). To examine lactate levels in inflamed skin, full thickness biopsies of IMQ-treated or control skin were cultured for 24 hours, in the presence or absence of glycolysis inhibitor 2DG, and measured lactate in the culture supernatant (FIG. 13A). Consistent with the organoid data, IMQ-treated skin culture supernatant had significantly higher levels of lactate than control skin, which was diminished upon treatment with 2DG (FIG. 13A). This production of lactate was largely driven by epithelial-specific glycolysis as IMQ-treated Slc2a1^{EKO} ex vivo cultures had significant reduction in lactate production compared to controls (FIG. 6K). To test whether limiting lactate itself could modulate cutaneous Type 17 response, it was sought to specifically limit lactate production and transport during IMQ-induced inflammation. Thus, topical treatment was performed with sodium oxamate (lactate dehydrogenase-A inhibitor) or syrosingopine (lactate transporters MCT1 and MCT4 inhibitor) during the course of IMQ treatment. Remarkably, inhibition of lactate production was sufficient to limit the cutaneous T cell numbers, Type 17 cytokines (Il17a, Il22, Il23a, Il1b, Tnfa) and epithelial thickness (FIGS. 6L-6M and 13B). It was also confirmed that interventional treatment with these inhibitors after establishment of inflammation at day 3 of IMQ treatment led to a decrease in the cutaneous Type 17 immunity (FIGS. 6N and 13C-13D). Taken together, these findings reveal epidermal HIF1α-induced glycolysis, and its substrate lactate, as the Achilles heel of inflammatory diseases and underscore the utility of therapeutically targeting metabolic pathways to modulate skin inflammation (FIG. 6O).

Unchecked crosstalk between immune and epithelial cells is known to drive inflammatory diseases. The inflammatory mediators of this communique, such as IL-23, IL-17A, and TNFα, have been extensively investigated and are the focus of therapeutic modulation (Ghoreschi et al., 2021). Yet, precisely how these inflammatory signals are interpreted by epithelia to induce pathology and if targeting epithelial programs alone is sufficient to curb disease is unclear. Here HIF1α was identified as a key regulator of epidermal dysfunction downstream of IL-17A signaling in PsO. In addition, it was found that HIF1α is also markedly activated in AD and HS, suggesting that this transcription factor may control epidermal pathological states across a range of inflammatory diseases associated with epithelial dysfunction.

Strikingly, both anti-IL-17A and anti-TNFα responsive patients downmodulated HIF1α, underscoring a role for this factor as a potential biomarker of therapeutic responsiveness. To move beyond association, HIF1α inhibition of primary PsO lesions was compared to the current SoC topical therapy, calcipotriene and betamethasone dipropionate (Kontochristopoulos et al., 2016). HIF1α inhibition resulted in a dramatic dampening of metabolic and epidermal genes, while SoC altered a much smaller fraction of factors. Thus, targeting HIF1α may also result in faster plaque clearance compared to SoC, which takes on average eight weeks and is only effective in 65-75% of patients.

The parallels between inflammatory remodeling of tissue and development remodeling are well appreciated, as developmental programs resurface in infections, inflammatory diseases, and cancers (Reynolds et al., 2021). Indeed, HIFs are essential for establishing the epidermal barrier in utero, and in the absence of HIF1α, HIF2a compensates in the developing epidermis (Wong et al., 2015). By contrast, a non-redundant role for HIF1α in inflammatory disease was found. In addition, Hif1a^{EKO} mice had no overt signs of barrier dysfunction or inflammation previously observed in the HIF1α/HIF2α double knockouts (Wong et al., 2015). Many of the genes controlled developmentally by HIF1α including terminal epidermal differentiation proteins, Flg and Lor, are augmented in IMQ inflammation in a HIF1α-dependent manner. In addition, dysplastic keratins and metabolic programs were also heightened in inflamed epidermis, indicating a highly context-specific transcriptional program induced by HIF1α. Accordingly, HIF1α hyperactivation alone is insufficient to instigate inflammatory disease, and so it was determined that its presence within the epithelium is necessary in two mouse models of psoriasiform inflammation.

In both mice and humans, HIF1α-controlled transcriptional programs, including glycolysis, clearly stratified patients who had received anti-IL-17A therapy from placebo and further delineated clinical responders from non-responders. These data raised the tantalizing possibility that inhibiting glycolysis in the epidermis could ameliorate disease via intrinsic modulation of epithelium. Indeed, inhibiting glycolysis alone restrained epidermal dysfunction and IMQ-mediated pathology. Given that HIF1α inhibition also led to diminished inflammation, it was questioned precisely how inhibiting metabolism in the epithelium could alter the behavior of inflammatory T cells. Because canonical regulators of Type 17 cells were not altered in the transcriptomics analysis, it was postulated that this effect may be due to a metabolic mediator.

Cancer cells famously rewire their metabolism through a phenomenon called the Warburg effect toward glycolysis to sustain their proliferative state. As such, the effects of glycolytic byproducts, in particular lactate, on tumor immune function have extensively been studied. Tumor cell-derived lactate signals directly into macrophages to direct their polarization to arginine-producing M2-like state (Colegio et al., 2014). Similarly, work from the Delgoff lab identified tumor-derived lactate as a source of fuel for tumor-associated regulatory T cells (Watson et al., 2021). Thus, lactate directs an immune-suppressive microenvironment in cancer. By contrast, limiting epithelial glycolysis in PsO curbed the inflammatory response, suggesting that lactate may also play a contextual role in promoting inflammation.

T cells are exquisitely sensitive to environment nutrient availability and adapt their metabolism to utilize physiological carbon sources for fuel. Effector CD8⁺ T cells, for instance, preferentially use lactate over glucose as a substrate for the TCA cycle (Kaymak et al., 2022). Th17 cells are known to be highly glycolic and high glucose environments can even induce Th17 cells contributing to autoimmune pathology, raising the question of how limiting epithelial glycolysis curbed the Type 17 inflammatory response (Wagner et al., 2021; Wu et al., 2020). It was found that HIF1α robustly augmented epithelial expression glucose transporter 1 (Glut1), and a transcriptional program of glycolysis, that is functionally necessary for epithelial pathology. Thus, inflamed epithelia, which outnumber T cells 10:1 in human PsO lesions, may preferentially consume glucose, creating a glucose limiting microenvironment in inflamed skin. Similarly, fibroblasts stimulated with IL-17 are known to rapidly upregulated glycolysis programs in inflamed lymph nodes and tumors, suggesting that immune cells must compete with the surrounding parenchymal cells for fuel (Majumder et al., 2019). Thus, like Tregs and CD8 T cells, Type 17 immune cells may have evolved also to use alternate carbon sources (e.g., lactate), to sustain their function in glucose limiting environments. Indeed, it was found that lactate was abundant in IMQ-inflamed skin and inhibiting lactate dehydrogenase a (LDHA) or the lactate transporters MCT1/4 protected mice from IMQ-induced inflammation. Future studies will help determine if the effects of lactate are mediated by direct effects on skin Type 17 cells and/or via antigen-presenting cells and if Type 17 immune cells can use lactate as a fuel source or if lactate exposure aggravates the inflammatory transcriptional response.

Overall, these findings underscore the pathological co-opting of the IL-17A-HIF1α signaling axis in PsO and further reveal that the program of glycolysis fuels disease states, both by intrinsically modulating epithelial behavior and extrinsically by augmenting T cell responses. This study establishes the proof of concept that modulating metabolic programs is sufficient to treat inflammatory disease and underscores the role of metabolic mediators in pathological immune-epithelial communication.

### Example 7. Testing Topical HIF1α siRNA in Skin Inflammation

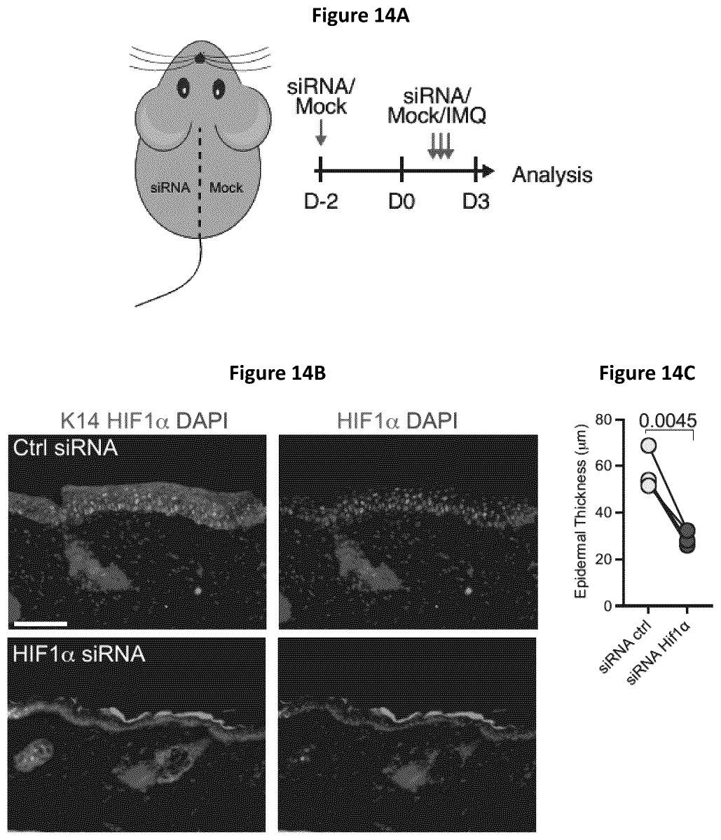

Murine SMART-pool HIF1α siRNA were purchased from Dharmacon (Sequences: UGAGAGAAAUGCUUACACA (SEQ ID NO: 35), GGAAAGAGAGUCAUAGAAC (SEQ ID NO: 36), UUACUGAGUUGAUGGGUUA (SEQ ID NO: 37), UUUAAUACCCUCCGAUUUA (SEQ ID NO: 38)). For topical application, 2.5-5 nM of each HIF1α or control non-targeting siRNA were combined with Lipofectamine 3000 and Vanicream and applied to dorsal skin of mice every 2 days. Mice were then subject to inflammation-inducing imiquimod (FIG. 14A). Skin was analyzed for signs of over pathology (thickening, flakiness, redness) and microscopic epidermal and immune pathology. In vivo HIF1α knockdown efficacy in the suprabasal epidermis was tested using immunofluorescence analysis of HIF1α protein (FIGS. 14B-14C). A loss of nuclear HIF1α expression (green round nuclei) was exhibited in the epidermis (K14+, red) in HIF1α siRNA-treated animals that correlates with reduced epidermal pathology.

### Example 8. Testing HIF1α Silencing by shRNA

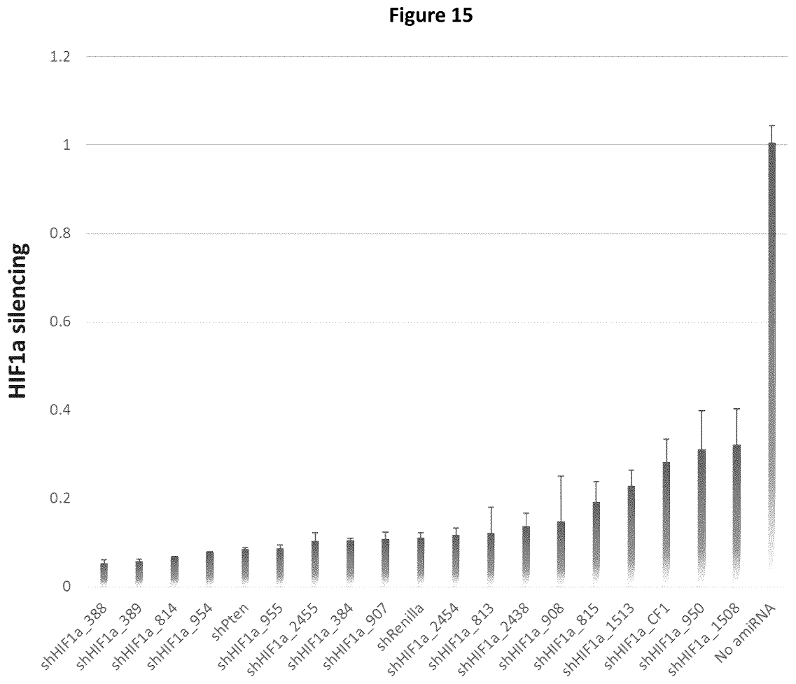

FIG. 15 shows shRNA knockdown efficiency of HIF1α using an HIF1α response element (HRE) Renilla luciferase reporter cell line. Briefly, shRNA efficacy in targeting HIF1α transcripts was assessed based on reporter activity relative to the negative control (no amiRNA). shRenilla and shPten were used as positive controls. Plots displayed reporter activity of controls and HIF1α shRNAs. The DNA templates for the antisense guide sequences in the shRNAs tested include: shHIF1a_2455 (TTAACTTGATCCAAAGCTCTGA, SEQ ID NO: 39), shHIF1a_388

shHIF1a_2455
(TTAACTTGATCCAAAGCTCTGA, SEQ ID NO: 39),
shHIF1a_388
(TGAGTAAAATCAAACACACTGT, SEQ ID NO: 40),
shHIFla_814
(TAATATTCATAAATTGAGCGGC, SEQ ID NO: 41),
shHIFla_955
(TTATATATGACAGTTGCTTGAG, SEQ ID NO: 42),
shHIFla_2438
(TCTGAGTAATTCTTCACCCTGC, SEQ ID NO: 43),
shHIFla_2454
(TAACTTGATCCAAAGCTCTGAG, SEQ ID NO: 44),
shHIFla_954
(TATATATGACAGTTGCTTGAGT, SEQ ID NO: 45),
shHIFla_1513
(TCAGGTGAACTTTGTCTAGTGC, SEQ ID NO: 46),
shHIF1a_389
(ATGAGTAAAATCAAACACACTG, SEQ ID NO: 47),
shHIF1a_384
(TAAAATCAAACACACTGTGTCC, SEQ ID NO: 48),
shHIF1a_813
(AATATTCATAAATTGAGCGGCC, SEQ ID NO: 49),
shHIFla_908
(TTTGGCAAGCATCCTGTACTGT, SEQ ID NO: 50),
shHIF1a_1508
(TGAACTTTGTCTAGTGCTTCCA, SEQ ID NO: 51),
shHIF1a_815
(ATAATATTCATAAATTGAGCGG, SEQ ID NO: 52),
shHIF1a_950
(TATGACAGTTGCTTGAGTTTCA, SEQ ID NO: 53),
shHIF1a_907
(TTGGCAAGCATCCTGTACTGTC, SEQ ID NO: 54),
and
shHIF1a_CF1
(TATATTCCTAAAATAATGCTTC, SEQ ID NO: 55).

Below are the methods used in the Examples described above.

Animals. The following mouse strains were purchased from The Jackson Laboratory: C57BL/6, Tg (KRT14-crc)1Amc/J, B6.129-Hif1a^{tm3Rsjo}/J, Slc2a1^{tm1.1Stma}/AbelJ. C57BL/6 mice were obtained from either Jackson Laboratories or Taconic Biosciences. Il17ra^{-/-} mice were obtained from Amgen Bioscience via an MTA. All animal studies were approved by NYU Grossman School of Medicine's Institutional Animal Care and Use Committee (IACUC). Mice were bred and maintained at New York University Langone Health Center under specific pathogen-free conditions in Association for Assessment and Accreditation of Laboratory Animal Care (AAALAC)-accredited facilities as well as housed in accordance with procedures outline in the National Research Council's Guide for the Care and Use of Laboratory Animals. Experiments were performed with IACUC-approved protocols. Age- and sex-matched controls were used in all experiments presented. Where appropriate, mice were randomly assigned to groups.

Human skin samples. Healthy, lesional, and non-lesional human skin samples were collected from donors whose characteristics are listed in Table 1, 2, and 3. Following anesthetization with 2% lidocaine injection, 4 mm full-thickness punch biopsies were performed on lesional skin, and matched non-lesional skin located contralaterally. In healthy volunteers, skin biopsy samples were obtained from the trunk and/or extremities. Post-therapy biopsies from secukinumab-treated patients were taken 5 to 10 mm away from the original biopsy site within the original lesion area, discerned by hyperpigmentation, and at contralateral non-lesional skin.

Human skin culture. 4 mm full-thickness punch biopsies from lesional psoriatic skin and healthy skin (foreskin) were cut into two pieces, secured with matrigel in 24-well plate and cultured within a maximum of 24-hours after removal from the patient. Each biopsy piece was subjected to different treatment conditions: (1) DMSO vehicle, (2) 10 μM of BAY87-2243, and (3) 500 nM of calcipotriol and 5 μM of betamethasone dipropionate (standard of care). The tissue was cultured in 24-well plates in 500 μl of Dulbecco's Modified Eagle Medium (DMEM) with 10% Fetal Bovine Serum (FBS). After 24 hours of culture, tissues were flash-frozen in liquid nitrogen and stored in −80° C. until RNA extraction. Total RNA was extracted using the RNeasy Fibrous Tissue Mini Kit (Qiagen) per manufacturer's instructions. Libraries were generated with Ribo-Zero Plus rRNA Depletion Kit (Illumina) and sequenced on Novaseq 6000 system with 100-bp paired-end reads.

Murine models of psoriatic inflammation. Topical imiquimod application: For imiquimod studies, seven- to eight-week-old mice in telogen (resting phase of hair cycle) were anesthetized with isoflurane prior to hair removal and imiquimod treatment on dorsal skin. 50 mg of imiquimod cream (5%, Perrigo) was applied to shaved dorsal skin, and application was repeated up to six days as previously described (van der Fits et al., 2009). Intradermal rmIL-23 injection: For recombinant murine (rm) IL-23 studies, the ear pinnae in 8-week-old mice were intradermally injected with either rmIL-23 (500 ng in 20 μl of PBS, carrier-free, PeproTech) or PBS control for five days.

Animal drug treatments. EdU pulse: Mice were injected intraperitoneally with EdU (50 μg/g) (Thermo Scientific) three hours prior to sacrifice. PIM HCl: Pimonidazole Hydrochloride (PIM HCl, hypoxyprobe) was injected intraperitoneally into mice (20 mg/kg) two hours prior to sacrifice. BAY 87-2243: BAY 87-2243 (2 mg/ml, Selleck Chemicals) was first dissolved in DMSO and then diluted in a water-based gel. This gel was topically applied to dorsal skin daily starting two days prior to imiquimod application and continuing in parallel with imiquimod application until analysis. Sodium Oxamate: Sodium oxamate (70 mg/ml, Cayman Chemicals) was first dissolved in H₂O and then diluted in a water-based gel. This gel was topically applied to dorsal skin daily starting either on the first or fourth day of imiquimod application and continuing in parallel with imiquimod application until analysis. Syrosingopine: Syrosingopine (10 mg/ml, Cayman Chemicals) was first dissolved in DMSO and then diluted in a water-based gel. This gel was topically applied to dorsal skin daily starting either on the first or fourth day of imiquimod application and continuing in parallel with imiquimod application until analysis.

Human skin immunofluorescence and image analyses. Four-micron paraffin-embedded sections were stained either with hematoxylin and eosin or with Akoya Biosciences® Opal™ multiplex automation kit on a Leica BondRX® autostainer, according to manufacturers' instructions. Semi-automated image acquisition was performed on a Vectra® Polaris multispectral imaging system. After whole slide scanning at 20× the tissue was manually outlined to select fields for spectral unmixing using InForm® version 2.4.11 software from Akoya Biosciences.

Murine skin immunofluorescence and image analyses. Immunofluorescence staining protocols were adapted from previously described methods (Konieczny et al., 2022). Tissue was fixed in 4% paraformaldehyde for 1 hour at 4° C., washed three times with PBS, and switched to 30% sucrose overnight. Following subsequent washes with PBS, tissue was embedded in optimal cutting temperature medium (OCT), frozen, cut into 10 μm thick sections, and placed on superfrost plus slides. Sections were blocked and stained with primary and secondary fluorescence-conjugated antibody. Nuclei were stained with DAPI. EdU reactions were performed according to the manufacturer's directions (Life Technologies). Pimonidazole Hydrochloride (PIM HCl) was detected by pretreating slides with a mouse-on-mouse immunodetection kit (Vector Laboratories) and then staining with biotin-anti-PIM adduct antibodies as per the manufacturer's guidelines (Hypoxyprobe). Data were analyzed using ImageJ software. Epidermal thickness was quantified by measuring K14⁺ thickness with an in-house ImageJ macro. Thickness of keratin 6 (K6) and glucose transporter 1 (Glut1) layer was quantified by measuring K6⁺K14⁺ and Glut1⁺K14⁺ thickness with an ImageJ macro. The number of CD3 and EdU positive cells was quantified using ImageJ software and normalized per dermis area and length of epidermis, respectively.

ImageJ was used to quantify Ndufa412 and glucose transporter 1 (Glut1) integrated density, a well-established method of measuring fluorescence intensity that accounts for differences in the area of the signal in epidermis. Images are shown with the pseudocolor Fire from ImageJ after they were contrasted equally and background was uniformly removed. For the complete list of immunofluorescence antibodies used, see Table 4.

TABLE 4
Key Resources.

| | | |
|---|---|---|
| REAGENT or RESOURCE | SOURCE | IDENTIFIER |

Antibodies

| | | |
|---|---|---|
| Rabbit Monoclonal anti-HIF1a | Cell Signaling | Cat# 36169S; |
| (clone D1S7W) | Technology | RRID: |
| | | AB_2799095 |
| Chicken Polyclonal | BioLegend | Cat# 906004, |
| anti-Keratin14 | | RRID: |
| | | AB_2616962 |
| Rabbit Monoclonal anti-CD3 | Abcam | Cat# ab16669; |
| (clone SP7) | | RRID: |
| | | AB_443425 |
| Rabbit Polyclonal anti-Keratin 6A | BioLegend | Cat# 905702; |
| (clone Poly19057) | | RRID: |
| | | AB_2734680 |
| Rat Monoclonal anti-Ki-67 | Thermo Fisher | Cat# 14-5698-80, |
| (clone SolA15) | Scientific | RRID: |
| | | AB_10853185 |
| Rat Monoclonal anti-I-A/I-E | BioLegend | Cat# 107601; |
| (clone M5/114.15.2) | | RRID: |
| | | AB_313316 |
| Rabbit Polyclonal anti-Keratin 10 | BioLegend | Cat# 905403; |
| | | RRID: |
| | | AB_2749902 |
| Rabbit Polyclonal human anti-IL17RC | LifeSpan | Cat#: LS-C400522 |
| | Biosciences | |
| Rabbit Monoclonal Cytokeratin 5 | Abcam | Cat#: ab52635; |
| (clone EP1601Y) | | RRID: |
| | | AB_1159462 |
| Rat Monoclonal anti-ROR gamma | Thermo Fisher | Cat# 14-6988-82, |
| (t) (clone AFKJS-9) | Scientific | RRID: |
| | | AB_1834475 |
| Rabbit Polyclonal anti-Ndufa4l2 | Thermo Fisher | Cat#: PA5-120264; |
| | Scientific | RRID: |
| | | AB_2913836 |
| Rabbit anti-Glut1 | Alpha Diagnostic | Cat# GT11-A, |
| | International | RRID: |
| | | AB_2895172 |

Biological samples

| | | |
|---|---|---|
| Skin Biopsies | NYU | N/A |
| Skin Biopsies | University of | N/A |
| | Michigan | |

Chemicals, peptides, and recombinant proteins

| | | |
|---|---|---|
| Imiquimod Cream, 5% | Perrigo | 45802-368-62 |
| BAY 87-2243 | Selleck Chemicals | S7309; CAS: |
| | | 1227158-85-1 |
| Calcipotriol | MedChem Express | HY-10001 |
| Betamethasone dipropionate | MedChem Express | HY-13571 |
| 2-Deoxy-D-glucose | Sigma-Aldrich | D8375; CAS: 154-17-6 |
| Recombinant Mouse IL-23 | R&D Systems | 1887-ML; |
| | | Accession #: |
| | | P43432 (p40) & |
| | | Q9EQ14 (p19) |
| Recombinant Mouse IL-17 | Peprotech | 200-17; |
| | | Accession #: Q16552 |
| Recombinant Mouse IL-22 | Peprotech | 210-22; |
| | | Accession #: Q9JJY9 |
| Sodium Oxamate | Cayman Chemicals | 19057; CAS: 565-73-1 |
| Syrosingopine | MedChem Express | HY-N4115 |

Critical commercial assays

| | | |
|---|---|---|
| Click-iT ™ EdU Cell Proliferation Kit | Thermo Fisher | C10340 |
| for Imaging, Alexa Fluor ™ 647 dye | Scientific | |
| M.O.M. ® (Mouse on Mouse) Blocking | Vector Laboratories | MKB-2213-1 |
| Reagent | | |
| Hypoxyprobe Kit | Hypoxyprobe | HP1-100Kit |
| Akoya Biosciences ® Opal ™ multiplex | Leica | ARD1001EA |
| automation kit | | |
| Leica Biosystems epitope retrieval 2 | Leica | AR9640 |
| solution | | |
| Prolong Gold Antifade Reagent | Invitrogen | P36930 |
| Visium Spatial Gene Expression Slide | 10X Genomics | 1000184 |
| & Reagent Kit | | |
| L-Lactate Assay Kit | Abcam | ab65331 |

Deposited data

| | | |
|---|---|---|
| Spatial transcriptomics of psoriatic | Castillo et al, in | Castillo et al, |
| lesions | press | in press |
| scRNA sequencing of human skin biopsies | Reynolds et al, 2021 | doi: |
| from healthy, psoriasis, and eczema | | 10.1126/science.ab |
| | | a6500 |
| RNA sequencing from psoriatic lesional | Krueger et al, 2019 | doi: 10.1016/ |
| biopsies from patients undergoing | | j.jaci.2019.04.029 |
| secukinumab or placebo treatment | | |

Experimental models: Organisms/strains

| | | |
|---|---|---|
| Mouse: C57BL/6J | The Jackson | JAX: 000664; |
| | Laboratory | RRID: IMSR_JAX: |
| | | 000664 |
| Mouse: C57BL/6J | Taconic Biosciences | B6-F |
| Mouse: Tg(KRT14-cre)1Amc/J | The Jackson | Jax: 004782; |
| | Laboratory | RRID: IMSR_JAX: |
| | | 004782 |
| Mouse: B6.129-Hif1a^{tm3Rsjo}/J | The Jackson | Jax: 007561; |
| | Laboratory | RRID: IMSR_JAX: |
| | | 007561 |
| Mouse: Slc2a1^{tml.1Stma}/AbelJ | The Jackson | Jax: 031871; |
| | Laboratory | RRID: IMSR_JAX: |
| | | 031871 |
| Mouse: IL17ra^{-/-} | Amgen Bioscience | MTA |

Oligonucleotides

| | | |
|---|---|---|
| Primers: see Table 5 | Described herein | N/A |

Software and algorithms

| | | |
|---|---|---|
| ImageJ | Schneider et al.⁷ | imagej.nih.gov/ij/ |
| R 4.2.2 | R Core Team (2022) | r-project.org/ |
| Bowtie2 | Langmead et al, 2009 | bowtie- |
| | | bio.sourceforge.net/ |
| | | bowtie2/index.shtml |
| Seurat v4.0 (R Package) | Stuart et al., 2019 | satijalab.org/seurat/ |
| EnrichR | Chen et al, 2013 | doi: 10.1186/ |
| | | 1471-2105-14-128. |
| InForm ® version 2.4.11 | Akoya Biosciences | |
| Bioconductor-GSVA | Hanzelmann et al, 2013 | bioconductor.org/ |
| | | packages/release/ |
| | | bioc/html/GSVA.html |
| Nmle | CRAN | cran.r-project.org/ |
| | | web/packages/nlme/ |
| | | index.html |
| harmony (R package) | Korsunsky et al., 2019 | github.com/ |
| | | immunogenomics/ |
| | | harmony |
| pheatmap v1.0.12 (R Package) | Kolde R., 2019 | CRAN.R-project.org/ |
| | | package = pheatmap |
| SCPubr | Blanco-Carmano E., 2022 | doi.org/10.1101/ |
| | | 2022.02.28.482303 |
| STAR v2.7.3a | Dobin et al., 2013 | code.google.com/p/ |
| | | rna-star/ |
| trimmomatic v0.36 | Bolger et al., 2014 | doi.org/10.1093/ |
| | | bioinformatics/btu170 |
| subread v1.6.3 | Liao et al., 2013 | doi.org/10.1093/ |
| | | nar/gkt214 |
| Seq-N-Slide | Dolgavel I. | igordot.github.io/sns |
| DESeq2 (R Package) | Love et al., 2014 | bioconductor.org/ |
| | | packages/release/ |
| | | bioc/html/DESeq2.html |

Analysis of publicly available scRNA-seq data. Publicly available single cell RNA seq data of healthy and inflamed skin was downloaded from the European Nucleotide Archive (ENA-ERP116319) in h5ad format and converted to h5seurat for downstream analysis in R. The full data set was rerun with the Seurat pipeline and harmonized by donor ID. Data was then subset to include only Keratinocyte subtypes and clustered at a resolution of 0.12 The top 40 Principal Components were used for clustering. The FindAllMarkers function was used to find markers for 7 generated clusters, after which 2 clusters were merged to give a final total of 5 clusters.

Spatial transcriptomics. Sample processing: Tissues were embedded in OCT and frozen in liquid nitrogen-chilled isopentane within 15 minutes of harvesting. 10 μm-thick cryosections were mounted onto the ST array slides. Tissue sections were fixed in methanol at −20° C. and then stained with hematoxylin and eosin. Brightfield images were taken on a Leica AT2 wide slide scanner at 20× resolution to be used downstream for gene mapping. The slides were inserted into cassettes that ensured leakproof wells for adding reagents. The tissues were permeabilized with Permeabilization Enzyme for 5 minutes at 37° C. as established by the tissue optimization protocol. RT Master Mix was added to the permeabilized tissue sections and incubated for 45 minutes at 53° C. Spatially barcoded full-length cDNA from poly-adenylated mRNA were generated followed by second strand synthesis at 650C for 15 minutes. The cDNA from each capture area was denatured and transferred to a corresponding tube for amplification and library construction. Samples were then sequenced on Illumina NovaSeq 6000. Spot selection and image alignment: Following probe cleavage, fluorescent images were taken on a Hamamatsu NanoZoomer whole slide fluorescence scanner. Brightfield and fluorescent images were manually aligned with Adobe Photoshop CS6 to identify the spatial orientation of the array with the tissue. Sequencing alignment and annotation: Sequencing output and histology images were processed with Space Ranger software (10× Genomics Visium). The Space Ranger mkfastq function was used for sample demultiplexing and converting spatial barcodes and reads into FASTQ format. The Space Ranger count function was then used to align reads to human genome (hg38) and further align the transcriptome with the microscopic slide image to generate barcode/UMI counts, feature spot matrices, cluster data, and gene-expression analyses. Clustering analysis of ST data: The Seurat R package was used for further clustering analyses. Samples were filtered out to remove spots with low depth of coverage (less than 200 detectable genes). Additionally, spots with >10% mitochondrial gene expression were filtered when clustering. To account for sequencing depth variance across tissue spots while controlling for technical artifacts, SCTransform was used to normalize gene expression by spots (Hafemeister & Satija, 2019). Samples were individually SCTransformed and then merged together. Merged samples were then batch corrected with Harmony integration and a resolution of 0.3 was used for clustering, Dimensionality reduction and clustering were run by performing PCA to generate graph-based cell clusters, and the top 40 PCs with a resolution of 0.3 were used, yielding 4 unique cell clusters. The markers genes were determined using FindAllMarkers function in Seurat, and the Wilcoxon rank-sum test was used for differential testing.

Transcriptional program response analyses to clinical trial data. Scoring methods: To evaluate if gene signatures derived from differential gene expression in human biopsy cultures or topical HIF1α inhibitor treatment in IMQ-treated murine model would be affected by anti-IL17 treatment, the genes down regulated by HIF1α inhibitor (HIF1α-signature) were projected into the longitudinal gene expression profiles of 31 psoriasis patients enrolled in a randomized clinical trial testing the efficacy of Secukinumab 300 mgs (n=22) versus Placebo (n=9) (Krueger et al., 2019). The overall expression/activity of the signatures was estimated using gene set variation analysis (GSVA). GSVA, a non-parametric and non-supervised method, estimated a sample-wise enrichment score for each gene set on the RCT cohorts. Such enrichment scores, representing overall gene set activity, were then modeled to evaluate the treatment effect and the association with treatment response. Modeling: Linear mixed-effect model (LMEM) was used with time and treatment (Secukinumab/Placebo) and its interaction as fixed effects a random intercept for each patient. Models were fitted using the nlme package in R, and estimated marginal means were obtained to present the average activity score for each treatment over time. The hypothesis of interest, i.e., longitudinal changes between treatment and estimated using contrasts. For patients treated with secukinumab, further compared were the secukinumab-induced changes between patients who were considered clinical responders after 12 weeks of treatment (>75% improvement in psoriasis activity index; PASI-75, n=15) and those of non-responders (n=7). In this case, the LMEM model included time, response, and its interaction as fixed effects.

Low-input bulk-RNA sequencing of human and murine skin. For human studies, skin biopsies were flash-frozen in liquid nitrogen following culture and treatment modalities. For murine studies, individual mice were used for RNA-sequencing; full-thickness skin biopsies of WT and Hif1a^{EKO} mice were flash frozen in liquid nitrogen. Both murine and human tissue biopsies were stored in −80° C. until RNA extraction. Total RNA was extracted using the RNeasy Fibrous Tissue Mini Kit (Qiagen) as per manufacturer's instructions. Libraries were generated with Ribo-Zero Plus rRNA Depletion Kit (Illumina) and sequenced on Novaseq 6000 system with 100-bp paired-end reads. Sequencing results were demultiplexed and converted to FASTQ format using Illumina bcl2fastq software. Reads were trimmed for adapter sequences using trimmomatic (version 0.36) in paired end mode with the minimal read length option set to 35 base pairs, trailing set to 5, and the sliding window set to 4:15. Sequencing reads were aligned to the mouse genome (mm10/GRCm38) and human genome (hg38) using the splice-aware STAR aligner (Dobin et al, 2013). Raw gene counts were tested for differential expression using negative binomial generalized linear models implemented by the DESeq2 R package (Love et al, 2014). Heatmaps of differentially expressed genes were made using Variance Stabilized Transformation (VST) counts with the pheatmap package in R (Kolde R., 2019). Pathways analysis of differentially expressed and up-regulated genes (log 2-fold change≥0.25, adjusted P value<0.1) was performed using EnrichR (Chen et al, 2013).

In vivo oxygen measurements. Tissue oxygen levels were measured using OxyLite probes (Oxford Optronix). The OxyLite NXpO2 Barer Fiber Sensor with a ˜250 mm tip was directly inserted into inflamed or healthy skin of an anesthetized mouse to continuously monitor oxygen pressure (pO2) for 15 to 20 min. pO2 levels were recorded and analyzed with LabChart Reader (Chart Version 8 for Windows; AD Instruments).

Organoid culture. Epithelial organoids were generated as previously described from the dorsal skin of ˜46-50-day old C56BL/6 mice (Konieczny et al., 2022). Following one day of culture at 21% O₂, rmIL-17A (500 ng/ml, Peprotech) or PBS was added to the organoid culture medium and replenished at day three. Organoids were harvested on day 6 with a nonenzymatic organoid harvesting solution (Cultrex). Quantitative PCR: Total RNA was extracted from organoids using RNeasy Plus Micro Kit (Qiagen), and equal amounts of RNA were reverse-transcribed using the superscript VILO cDNA synthesis kit (Invitrogen). Analytes were normalized using the housekeeping gene Actb. For the complete list of quantitative PCR primers, refer to Table 5.

TABLE 5
Primer sequences used for quantitative PCR.

| | | SEQ | | SEQ |
|---|---|---|---|---|
| | Forward Primer | ID | Reverse Primer | ID |
| Gene | (5′ -> 3′) | NO | (5′ -> 3′) | NO |
| Actb | ATCCTGGCCT | 1 | AAACGCAGCT | 2 |
| | CACTGTCCAC | | CAGTAACAGT | |
| | CTTC | | CCGC | |
| Hif1a | ACCTTCATCG | 3 | CTGTTAGGCT | 4 |
| | GAAACTCCAA | | GGGAAAAGTT | |
| | AG | | AGG | |
| Ndufa4l2 | AAAGACACCC | 5 | TGTAGTCGGT | 6 |
| | TGGGCTCATC | | TGAAACGGCA | |
| Nxph4 | CCTCTTGCTC | 7 | GACGCAAGTC | 8 |
| | TTTGGTCCTT | | TAGGTACTGG | |
| Ak4 | AAAGGATCGC | 9 | TCGGGAATCC | 10 |
| | CCAGAACTTT | | ATCTAACAGC | |
| Fam162a | CCCAAAACTC | 11 | CTTCACCCGG | 12 |
| | CAACCCAGTC | | AGCTTGTTCT | |
| | T | | | |
| Higd1a | CTGTGTTGGT | 13 | GCTGGCTTCT | 14 |
| | TGACATTGTG | | GATTGGCT | |
| | AT | | | |
| Slc2a1 | TGCAGTTCGG | 15 | GTAGCGGTGG | 16 |
| | CTATAACACT | | TTCCATGTTT | |
| Ki67 | AGAGCTAACT | 17 | TCAATACTCC | 18 |
| | TGCGCTGACT | | TTCCAAACAG | |
| | | | GCA | |
| Klf4 | GACTAACCGT | 19 | GTCTAGGTCC | 20 |
| | TGGCGTGAGG | | AGGAGGTCGT | |
| Keratin | AAGGTCATGG | 21 | CAGCATGTAG | 22 |
| 14 | ATGTGCACGA | | CAGCTTTAGT | |
| | T | | TCTTG | |
| Keratin | TTCTCTCCGG | 23 | AGCACGTTGG | 24 |
| 10 | AAACGAGAAG | | CATTGTCAGT | |
| | GTGA | | T | |
| Keratin | CAACATCATA | 25 | GAGGAAGCCA | 26 |
| 6a | ACCCTCCCTG | | AGAGCATCAA | |
| | TC | | | |
| Keratin | CTCTGAGCTC | 27 | GAGGACTGAC | 28 |
| 6b | ACATCACGAT | | ACACACTTTA | |
| | TC | | CC | |
| Keratin | TGAGATGAGG | 29 | TGCGGTTGCT | 30 |
| 16 | GACCAGTATG | | CTGGATTAG | |
| | A | | | |
| Keratin | CCACCATGCA | 31 | GGTACCAATC | 32 |
| 17 | GAACCTCAA | | TCGGATCTTC | |
| | | | AC | |
| IVL | TCCCTCCTGT | 33 | CAGTCTTGAG | 34 |
| | GAGTTTGTTT | | AGGTCCCTGA | |
| | G | | | |

Lactate Secretion Assay of ex vivo murine back skin culture. 4 mm full-thickness punch biopsies from IMQ-treated or healthy murine skin were secured with matrigel in 12-well plate and cultured for 24 hours in 400 μl of Dulbecco's Modified Eagle Medium (DMEM) with 10% Fetal Bovine Serum (FBS). For inhibitor studies, cultures were treated with 2-DG (50 mM). At the end of 24 hours, supernatant was collected, centrifuged at 1000 g to pellet debris, and stored in −80C until further use. Lactate was measured using the colorimetric L-Lactate Assay Kit (Abcam).

Statistics. Data in FIGS. 2-6, 7, and 10-13 are presented as mean±SEM. Group sizes were determined on the basis of the results of preliminary experiments. Mice were assigned at random to groups. Experiments were not performed in a blinded fashion. Statistical significance for each experiment was determined as shown in the figure legends, where n=the number of independent biological replicates (animals, unless noted as cells) per group, and N=the number of independent experimental replicates. Statistical significance of bulk RNA-seq data in FIGS. 4, 6, 9, and 12 was calculated using an adjusted P value cutoff<0.1 in the DESeq2 R package. Statistical analyses were performed in Prism (GraphPad), DESeq2, or R. No data points were excluded.

### REFERENCES

Canavese, M., Altruda, F., Ruzicka, T., and Schauber, J. (2010). Vascular endothelial growth factor (VEGF) in the pathogenesis of psoriasis—a possible target for novel therapies? J Dermatol Sci 58, 171-176.
Castillo, R. L., Sidhu, I., Dolgalev, I., Chu, T., Prystupa, A., Subudhi, I., Yan, D., Konieczny, P., Hsieh, B., Haberman, R. H., et al. (2023). Spatial transcriptomics stratifies psoriatic disease severity by emergent cellular ecosystems. Sci Immunol 8, eabq7991.
Chan, J. R., Blumenschein, W., Murphy, E., Diveu, C., Wiekowski, M., Abbondanzo, S., Lucian, L., Geissler, R., Brodie, S., Kimball, A. B., et al. (2006). IL-23 stimulates epidermal hyperplasia via TNF and IL-20R2-dependent mechanisms with implications for psoriasis pathogenesis. J Exp Med 203, 2577-2587.
Cheng, J. B., Sedgewick, A. J., Finnegan, A. I., Harirchian, P., Lee, J., Kwon, S., Fassett, M. S., Golovato, J., Gray, M., Ghadially, R., et al. (2018). Transcriptional Programming of Normal and Inflamed Human Epidermis at Single-Cell Resolution. Cell Rep 25, 871-883.
Colegio, O. R., Chu, N. Q., Szabo, A. L., Chu, T., Rhebergen, A. M., Jairam, V., Cyrus, N., Brokowski, C. E., Eisenbarth, S. C., Phillips, G. M., et al. (2014). Functional polarization of tumour-associated macrophages by tumour-derived lactic acid. Nature 513, 559-563.
Dai, X., Murakami, M., Shiraishi, K., Muto, J., Tohyama, M., Mori, H., Utsunomiya, R., and Sayama, K. (2022). EGFR ligands synergistically increase IL-17A-induced expression of psoriasis signature genes in human keratinocytes via IkappaBzeta and Bcl3. Eur J Immunol 52, 994-1005.
Dang, E. V., Barbi, J., Yang, H. Y., Jinasena, D., Yu, H., Zheng, Y., Bordman, Z., Fu, J., Kim, Y., Yen, H. R., et al. (2011). Control of T (H) 17/T (reg) balance by hypoxia-inducible factor 1. Cell 146, 772-784.
Ellinghaus, P., Heisler, I., Unterschemmann, K., Haerter, M., Beck, H., Greschat, S., Ehrmann, A., Summer, H., Flamme, I., Ochme, F., et al. (2013). BAY 87-2243, a highly potent and selective inhibitor of hypoxia-induced gene activation has antitumor activities by inhibition of mitochondrial complex I. Cancer Med 2, 611-624.
Eming, S. A., Wynn, T. A., and Martin, P. (2017). Inflammation and metabolism in tissue repair and regeneration. Science 356, 1026-1030.
Ghoreschi, K., Balato, A., Enerback, C., and Sabat, R. (2021). Therapeutics targeting the IL-23 and IL-17 pathway in psoriasis. Lancet 397, 754-766.
Griffiths, C. E. M., Armstrong, A. W., Gudjonsson, J. E., and Barker, J. (2021). Psoriasis. Lancet 397, 1301-1315.
Guenin-Mace, L., Konieczny, P., and Naik, S. (2022). Immune-Epithelial Cross Talk in Regeneration and Repair. Annu Rev Immunol.
Guttman-Yassky, E., and Krueger, J. G. (2017). Atopic dermatitis and psoriasis: two different immune diseases or one spectrum? Curr Opin Immunol 48, 68-73.
Jantsch, J., Wiese, M., Schodel, J., Castiglione, K., Glasner, J., Kolbe, S., Mole, D., Schleicher, U., Eckardt, K. U., Hensel, M., et al. (2011). Toll-like receptor activation and hypoxia use distinct signaling pathways to stabilize hypoxia-inducible factor 1alpha (HIF1A) and result in differential HIF1A-dependent gene expression. J Leukoc Biol 90, 551-562.
Kanehisa, M., and Goto, S. (2000). KEGG: kyoto encyclopedia of genes and genomes. Nucleic Acids Res 28, 27-30.
Kaymak, I., Luda, K. M., Duimstra, L. R., Ma, E. H., Longo, J., Dahabich, M. S., Faubert, B., Oswald, B. M., Watson, M. J., Kitchen-Goosen, S. M., et al. (2022). Carbon source availability drives nutrient utilization in CD8 (+) T cells. Cell Metab 34, 1298-1311 e1296.
Konieczny, P., Xing, Y., Sidhu, I., Subudhi, I., Mansfield, K. P., Hsieh, B., Biancur, D. E., Larsen, S. B., Cammer, M., Li, D., et al. (2022). Interleukin-17 governs hypoxic adaptation of injured epithelium. Science 377, eabg9302.
Kontochristopoulos, G., Kouris, A., Chantzaras, A., Petridis, A., and Yfantopoulos, J. (2016). Improvement of health-related quality of life and adherence to treatment with calcipotriol-betamethasone dipropionate gel in patients with psoriasis vulgaris. An Bras Dermatol 91, 160-166.
Krueger, J. G., Wharton, K. A., Jr., Schlitt, T., Suprun, M., Torene, R. I., Jiang, X., Wang, C. Q., Fuentes-Duculan, J., Hartmann, N., Peters, T., et al. (2019). IL-17A inhibition by secukinumab induces early clinical, histopathologic, and molecular resolution of psoriasis. J Allergy Clin Immunol 144, 750-763.
Lowes, M. A., Bowcock, A. M., and Krueger, J. G. (2007). Pathogenesis and therapy of psoriasis. Nature 445, 866-873.
Majumder, S., Amatya, N., Revu, S., Jawale, C. V., Wu, D., Rittenhouse, N., Menk, A., Kupul, S., Du, F., Raphael, I., et al. (2019). IL-17 metabolically reprograms activated fibroblastic reticular cells for proliferation and survival. Nat Immunol 20, 534-545.
McGeachy, M. J., Cua, D. J., and Gaffen, S. L. (2019). The IL-17 Family of Cytokines in Health and Disease. Immunity 50, 892-906.
Moncada, R., Barkley, D., Wagner, F., Chiodin, M., Devlin, J. C., Baron, M., Hajdu, C. H., Simeone, D. M., and Yanai, I. (2020). Integrating microarray-based spatial transcriptomics and single-cell RNA-seq reveals tissue architecture in pancreatic ductal adenocarcinomas. Nat Biotechnol.
Nograles, K. E., Zaba, L. C., Guttman-Yassky, E., Fuentes-Duculan, J., Suarez-Farinas, M., Cardinale, I., Khatcherian, A., Gonzalez, J., Pierson, K. C., White, T. R., et al. (2008). Th17 cytokines interleukin (IL)-17 and IL-22 modulate distinct inflammatory and keratinocyte-response pathways. Br J Dermatol 159, 1092-1102.
Rabinowitz, J. D., and Enerback, S. (2020). Lactate: the ugly duckling of energy metabolism. Nat Metab 2, 566-571.
Reynolds, G., Vegh, P., Fletcher, J., Poyner, E. F. M., Stephenson, E., Goh, I., Botting, R. A., Huang, N., Olabi, B., Dubois, A., et al. (2021). Developmental cell programs are co-opted in inflammatory skin disease. Science 371.
Rogalski, C. (2015). Calcipotriol/betamethasone for the treatment of psoriasis: efficacy, safety, and patient acceptability. Psoriasis (Auckl) 5, 97-107.
Sano, S., Chan, K. S., Carbajal, S., Clifford, J., Peavey, M., Kiguchi, K., Itami, S., Nickoloff, B. J., and DiGiovanni, J. (2005). Stat3 links activated keratinocytes and immunocytes required for development of psoriasis in a novel transgenic mouse model. Nat Med 11, 43-49.
Sano, S., Itami, S., Takeda, K., Tarutani, M., Yamaguchi, Y., Miura, H., Yoshikawa, K., Akira, S., and Takeda, J. (1999). Keratinocyte-specific ablation of Stat3 exhibits impaired skin remodeling, but does not affect skin morphogenesis. EMBO J 18, 4657-4668.
Schonthaler, H. B., Guinea-Viniegra, J., Wculek, S. K., Ruppen, I., Ximenez-Embun, P., Guio-Carrion, A., Navarro, R., Hogg, N., Ashman, K., and Wagner, E. F. (2013). S100A8-S100A9 protein complex mediates psoriasis by regulating the expression of complement factor C3. Immunity 39, 1171-1181.
Semenza, G. L. (2003). Targeting HIF-1 for cancer therapy. Nat Rev Cancer 3, 721-732.
Semenza, G. L. (2012). Hypoxia-inducible factors in physiology and medicine. Cell 148, 399-408.
Shao, S., Tsoi, L. C., Swindell, W. R., Chen, J., Uppala, R., Billi, A. C., Xing, X., Zeng, C., Sarkar, M. K., Wasikowski, R., et al. (2021). IRAK2 Has a Critical Role in Promoting Feed-Forward Amplification of Epidermal Inflammatory Responses. J Invest Dermatol 141, 2436-2448.
Stinco, G., Balato, N., Buligan, C., Campanati, A., Dastoli, S., Di Meo, N., Gisondi, P., Lacarubba, F., Musumeci, M. L., Napolitano, M., et al. (2019). A multicenter retrospective case-control study on Suspension of TNF-inhibitors and Outcomes in Psoriatic patients (STOP study). G Ital Dermatol Venereol 154, 392-399.
Travis K Hughes, M. H. W. I., Todd M Gierahn, Tran Do, David Weiss, Priscilla R. Andrade, Feiyang Ma, Bruno J. de Andrade Silva, Shuai Shao, Lam C Tsoi, Jose Ordovas-Montanes, Johann E Gudjonsson, Robert L Modlin, J Christopher Love, Alex K Shalek (2019). Highly Efficient, Massively-Parallel Single-Cell RNA-Seq Reveals Cellular States and Molecular Features of Human Skin Pathology. bioRxiv.
van der Fits, L., Mourits, S., Voerman, J. S. A., Kant, M., Boon, L., Laman, J. D., Cornelissen, F., Mus, A. M., Florencia, E., Prens, E. P., et al. (2009). Imiquimod-Induced Psoriasis-Like Skin Inflammation in Mice Is Mediated via the IL-23/IL-17 Axis. Journal of Immunology 182, 5836-5845.
Wagner, A., Wang, C., Fessler, J., DeTomaso, D., Avila-Pacheco, J., Kaminski, J., Zaghouani, S., Christian, E., Thakore, P., Schellhaass, B., et al. (2021). Metabolic modeling of single Th17 cells reveals regulators of autoimmunity. Cell 184, 4168-4185 €4121.
Watson, M. J., Vignali, P. D. A., Mullett, S. J., Overacre-Delgoffe, A. E., Peralta, R. M., Grebinoski, S., Menk, A. V., Rittenhouse, N. L., DePeaux, K., Whetstone, R. D., et al. (2021). Metabolic support of tumour-infiltrating regulatory T cells by lactic acid. Nature 597, 645-651.
Wong, W. J., Richardson, T., Seykora, J. T., Cotsarelis, G., and Simon, M. C. (2015). Hypoxia-inducible factors regulate filaggrin expression and epidermal barrier function. J Invest Dermatol 135, 454-461.
Wu, K., Zhou, K., Wang, Y., Zhou, Y., Tian, N., Wu, Y., Chen, D., Zhang, D., Wang, X., Xu, H., et al. (2016). Stabilization of HIF-1alpha by FG-4592 promotes functional recovery and neural protection in experimental spinal cord injury. Brain Res 1632, 19-26.
Wu, L., Hollinshead, K. E. R., Hao, Y., Au, C., Krochling, L., Ng, C., Lin, W. Y., Li, D., Silva, H. M., Shin, J., et al. (2020). Niche-Selective Inhibition of Pathogenic Th17 Cells by Targeting Metabolic Redundancy. Cell 182, 641-654 e620.a
Zaba, L. C., Cardinale, I., Gilleaudeau, P., Sullivan-Whalen, M., Suarez-Farinas, M., Fuentes-Duculan, J., Novitskaya, I., Khatcherian, A., Bluth, M. J., Lowes, M. A., et al. (2007). Amelioration of epidermal hyperplasia by TNF inhibition is associated with reduced Th17 responses. J Exp Med 204, 3183-3194.
Zambrano, A., et al. (2019). Glut 1 in Cancer Cells and the Inhibitory Action of Resveratrol as A Potential Therapeutic Strategy. Int J Mol Sci 20, 3374.
Zhang, Y., et al. (2022). Targeting Glucose Metabolism Enzymes in Cancer Treatment: Current and Emerging Strategies. Cancers (Basel) 14, 4568.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method of treating an inflammatory skin disease in a subject in need thereof, comprising administering to the subject an effective amount of an inhibitor of Hypoxia Inducible Factor 1α (HIF1α).

2. The method of claim 1, wherein the inflammatory skin disease is selected from psoriasis, atopic dermatitis, skin rash, hidradenitis suppurativa, actinic keratosis, seborrheic dermatitis, cutaneous lupus, and lichen planus.

3. The method of claim 2, wherein the inflammatory skin disease is psoriasis.

4. The method of claim 3, wherein the psoriasis is selected from plaque psoriasis, guttate psoriasis, pustular psoriasis, and inverse psoriasis.

5. The method of claim 1, wherein the inhibitor of HIF1α inhibits expression or function of HIF1α protein.

6. The method of claim 1, wherein the inhibitor of HIF1α is an interfering nucleic acid molecule, a ribozyme, a gene editing molecule, or a small molecule.

7. The method of claim 6, wherein the interfering nucleic acid molecule is an siRNA, an shRNA, a miRNA, or an antisense oligonucleotide.

8. The method of claim 7, wherein the interfering nucleic acid molecule is an siRNA or shRNA.

9. The method of claim 8, wherein the siRNA comprises a nucleotide sequence
(SEQ ID NO: 35)
UGAGAGAAAUGCUUACACA,
(SEQ ID NO: 36)
GGAAAGAGAGUCAUAGAAC,
(SEQ ID NO: 37)
UUACUGAGUUGAUGGGUUA,
or
(SEQ ID NO: 38)
UUUAAUACCCUCCGAUUUA.

10.-11. (canceled)

12. The method of claim 8, wherein the shRNA comprises a nucleotide sequence UUAACUUGAUCCAAAGCUCUGA (SEQ ID NO: 56),
UGAGUAAAAUCAAACACACUGU (SEQ ID NO: 57),
UAAUAUUCAUAAAUUGAGCGGC (SEQ ID NO: 58),
UUAUAUAUGACAGUUGCUUGAG (SEQ ID NO: 59),
UCUGAGUAAUUCUUCACCCUGC (SEQ ID NO: 60),
UAACUUGAUCCAAAGCUCUGAG (SEQ ID NO: 61),
UAUAUAUGACAGUUGCUUGAGU (SEQ ID NO: 62),
UCAGGUGAACUUUGUCUAGUGC (SEQ ID NO: 63),
AUGAGUAAAAUCAAACACACUG (SEQ ID NO: 64),
UAAAAUCAAACACACUGUGUCC (SEQ ID NO: 65),
AAUAUUCAUAAAUUGAGCGGCC (SEQ ID NO: 66),
UUUGGCAAGCAUCCUGUACUGU (SEQ ID NO: 67),
UGAACUUUGUCUAGUGCUUCCA (SEQ ID NO: 68),
AUAAUAUUCAUAAAUUGAGCGG (SEQ ID NO: 69),
UAUGACAGUUGCUUGAGUUUCA (SEQ ID NO: 70),
UUGGCAAGCAUCCUGUACUGUC (SEQ ID NO: 71), or
UAUAUUCCUAAAAUAAUGCUUC (SEQ ID NO: 72).

13. The method of claim 8, wherein the siRNA or shRNA is chemically modified.

14. The method of claim 6, wherein the small molecule is selected from Mitoquinone mesylate, TLC-388 HCl, HS-111, IDF-11774, LW-1564, NXC-828, BAY-87-2243, Camptothecin, DFN-529, FG-2216, 2-Methoxyestradiol, AG-311, BACPTDP, BAY-97-2243, DD-001, Drupanol, GBH-1a, LS-081, Palomids, PX-478, RY-10-4, SR-16388, XL-388, and YC-1.

15. The method of claim 1, wherein the inhibitor of HIF1α is administered topically to an affected skin area.

16. The method of claim 15, wherein the inhibitor of HIF1α is administered to the suprabasal layer of the epidermis in the affected skin area.

17. The method of claim 1, wherein the inhibitor of HIF1α is formulated for topical administration.

18. The method of claim 17, wherein the inhibitor of HIF1α is formulated for topical administration to the suprabasal layer of the epidermis.

19. The method of claim 17, wherein the inhibitor of HIF1α is formulated in nanoparticles or liposomes.

20. The method of claim 19, wherein the nanoparticles or liposomes facilitate targeted delivery of the inhibitor of HIF1α to the suprabasal layer of the epidermis.

21. The method of claim 1, further comprising administering to the subject an effective amount of an anti-psoriasis agent.

22. The method of claim 21, wherein the anti-psoriasis agent is selected from creams, ointments, corticosteroids, coal tar, anthralin, vitamin D analogues, retinoids, calcineurin inhibitors, light therapies, retinoids, methotrexate, and cyclosporine.

23. (canceled)

24. A topical formulation comprising an inhibitor of Hypoxia Inducible Factor 1α (HIF1α) and a pharmaceutically acceptable carrier or excipient.

25.-39. (canceled)

40. A method of treating an inflammatory skin disease in a subject in need thereof, comprising administering to the subject an effective amount of an inhibitor of glucose transporter 1 (Glut1).

41.-62. (canceled)

63. A topical formulation comprising an inhibitor of glucose transporter 1 (Glut1) and a pharmaceutically acceptable carrier or excipient.

64.-78. (canceled)


SEQUENCE LISTING (73 sequences)

SEQ ID NO: 1 - DNA, 24 bp, synthetic construct
  source 1..24: mol_type = other DNA
atcctggcctcactgtccaccttc

SEQ ID NO: 2 - DNA, 24 bp, synthetic construct
  source 1..24: mol_type = other DNA
aaacgcagctcagtaacagtccgc

SEQ ID NO: 3 - DNA, 22 bp, synthetic construct
  source 1..22: mol_type = other DNA
accttcatcggaaactccaaag

SEQ ID NO: 4 - DNA, 23 bp, synthetic construct
  source 1..23: mol_type = other DNA
ctgttaggctgggaaaagttagg

SEQ ID NO: 5 - DNA, 20 bp, synthetic construct
  source 1..20: mol_type = other DNA
aaagacaccctgggctcatc

SEQ ID NO: 6 - DNA, 20 bp, synthetic construct
  source 1..20: mol_type = other DNA
tgtagtcggttgaaacggca

SEQ ID NO: 7 - DNA, 20 bp, synthetic construct
  source 1..20: mol_type = other DNA
cctcttgctctttggtcctt

SEQ ID NO: 8 - DNA, 20 bp, synthetic construct
  source 1..20: mol_type = other DNA
gacgcaagtctaggtactgg

SEQ ID NO: 9 - DNA, 20 bp, synthetic construct
  source 1..20: mol_type = other DNA
aaaggatcgcccagaacttt

SEQ ID NO: 10 - DNA, 20 bp, synthetic construct
  source 1..20: mol_type = other DNA
tcgggaatccatctaacagc

SEQ ID NO: 11 - DNA, 21 bp, synthetic construct
  source 1..21: mol_type = other DNA
cccaaaactccaacccagtct

SEQ ID NO: 12 - DNA, 20 bp, synthetic construct
  source 1..20: mol_type = other DNA
cttcacccggagcttgttct

SEQ ID NO: 13 - DNA, 22 bp, synthetic construct
  source 1..22: mol_type = other DNA
ctgtgttggttgacattgtgat

SEQ ID NO: 14 - DNA, 18 bp, synthetic construct
  source 1..18: mol_type = other DNA
gctggcttctgattggct

SEQ ID NO: 15 - DNA, 20 bp, synthetic construct
  source 1..20: mol_type = other DNA
tgcagttcggctataacact

SEQ ID NO: 16 - DNA, 20 bp, synthetic construct
  source 1..20: mol_type = other DNA
gtagcggtggttccatgttt

SEQ ID NO: 17 - DNA, 20 bp, synthetic construct
  source 1..20: mol_type = other DNA
agagctaacttgcgctgact

SEQ ID NO: 18 - DNA, 23 bp, synthetic construct
  source 1..23: mol_type = other DNA
tcaatactccttccaaacaggca

SEQ ID NO: 19 - DNA, 20 bp, synthetic construct
  source 1..20: mol_type = other DNA
gactaaccgttggcgtgagg

SEQ ID NO: 20 - DNA, 20 bp, synthetic construct
  source 1..20: mol_type = other DNA
gtctaggtccaggaggtcgt

SEQ ID NO: 21 - DNA, 21 bp, synthetic construct
  source 1..21: mol_type = other DNA
aaggtcatggatgtgcacgat

SEQ ID NO: 22 - DNA, 25 bp, synthetic construct
  source 1..25: mol_type = other DNA
cagcatgtagcagctttagttcttg

SEQ ID NO: 23 - DNA, 24 bp, synthetic construct
  source 1..24: mol_type = other DNA
ttctctccggaaacgagaaggtga

SEQ ID NO: 24 - DNA, 21 bp, synthetic construct
  source 1..21: mol_type = other DNA
agcacgttggcattgtcagtt

SEQ ID NO: 25 - DNA, 22 bp, synthetic construct
  source 1..22: mol_type = other DNA
caacatcataaccctccctgtc

SEQ ID NO: 26 - DNA, 20 bp, synthetic construct
  source 1..20: mol_type = other DNA
gaggaagccaagagcatcaa

SEQ ID NO: 27 - DNA, 22 bp, synthetic construct
  source 1..22: mol_type = other DNA
ctctgagctcacatcacgattc

SEQ ID NO: 28 - DNA, 22 bp, synthetic construct
  source 1..22: mol_type = other DNA
gaggactgacacacactttacc

SEQ ID NO: 29 - DNA, 21 bp, synthetic construct
  source 1..21: mol_type = other DNA
tgagatgagggaccagtatga

SEQ ID NO: 30 - DNA, 19 bp, synthetic construct
  source 1..19: mol_type = other DNA
tgcggttgctctggattag

SEQ ID NO: 31 - DNA, 19 bp, synthetic construct
  source 1..19: mol_type = other DNA
ccaccatgcagaacctcaa

SEQ ID NO: 32 - DNA, 22 bp, synthetic construct
  source 1..22: mol_type = other DNA
ggtaccaatctcggatcttcac

SEQ ID NO: 33 - DNA, 21 bp, synthetic construct
  source 1..21: mol_type = other DNA
tccctcctgtgagtttgtttg

SEQ ID NO: 34 - DNA, 20 bp, synthetic construct
  source 1..20: mol_type = other DNA
cagtcttgagaggtccctga

SEQ ID NO: 35 - RNA, 19 nt, synthetic construct
  source 1..19: mol_type = other RNA
tgagagaaatgcttacaca

SEQ ID NO: 36 - RNA, 19 nt, synthetic construct
  source 1..19: mol_type = other RNA
ggaaagagagtcatagaac

SEQ ID NO: 37 - RNA, 19 nt, synthetic construct
  source 1..19: mol_type = other RNA
ttactgagttgatgggtta

SEQ ID NO: 38 - RNA, 19 nt, synthetic construct
  source 1..19: mol_type = other RNA
tttaataccctccgattta

SEQ ID NO: 39 - DNA, 22 bp, synthetic construct
  source 1..22: mol_type = other DNA
ttaacttgatccaaagctctga

SEQ ID NO: 40 - DNA, 22 bp, synthetic construct
  source 1..22: mol_type = other DNA
tgagtaaaatcaaacacactgt

SEQ ID NO: 41 - DNA, 22 bp, synthetic construct
  source 1..22: mol_type = other DNA
taatattcataaattgagcggc

SEQ ID NO: 42 - DNA, 22 bp, synthetic construct
  source 1..22: mol_type = other DNA
ttatatatgacagttgcttgag

SEQ ID NO: 43 - DNA, 22 bp, synthetic construct
  source 1..22: mol_type = other DNA
tctgagtaattcttcaccctgc

SEQ ID NO: 44 - DNA, 22 bp, synthetic construct
  source 1..22: mol_type = other DNA
taacttgatccaaagctctgag

SEQ ID NO: 45 - DNA, 22 bp, synthetic construct
  source 1..22: mol_type = other DNA
tatatatgacagttgcttgagt

SEQ ID NO: 46 - DNA, 22 bp, synthetic construct
  source 1..22: mol_type = other DNA
tcaggtgaactttgtctagtgc

SEQ ID NO: 47 - DNA, 22 bp, synthetic construct
  source 1..22: mol_type = other DNA
atgagtaaaatcaaacacactg

SEQ ID NO: 48 - DNA, 22 bp, synthetic construct
  source 1..22: mol_type = other DNA
taaaatcaaacacactgtgtcc

SEQ ID NO: 49 - DNA, 22 bp, synthetic construct
  source 1..22: mol_type = other DNA
aatattcataaattgagcggcc

SEQ ID NO: 50 - DNA, 22 bp, synthetic construct
  source 1..22: mol_type = other DNA
tttggcaagcatcctgtactgt

SEQ ID NO: 51 - DNA, 22 bp, synthetic construct
  source 1..22: mol_type = other DNA
tgaactttgtctagtgcttcca

SEQ ID NO: 52 - DNA, 22 bp, synthetic construct
  source 1..22: mol_type = other DNA
ataatattcataaattgagcgg

SEQ ID NO: 53 - DNA, 22 bp, synthetic construct
  source 1..22: mol_type = other DNA
tatgacagttgcttgagtttca

SEQ ID NO: 54 - DNA, 22 bp, synthetic construct
  source 1..22: mol_type = other DNA
ttggcaagcatcctgtactgtc

SEQ ID NO: 55 - DNA, 22 bp, synthetic construct
  source 1..22: mol_type = other DNA
tatattcctaaaataatgcttc

SEQ ID NO: 56 - RNA, 22 nt, synthetic construct
  source 1..22: mol_type = other RNA
ttaacttgatccaaagctctga

SEQ ID NO: 57 - RNA, 22 nt, synthetic construct
  source 1..22: mol_type = other RNA
tgagtaaaatcaaacacactgt

SEQ ID NO: 58 - RNA, 22 nt, synthetic construct
  source 1..22: mol_type = other RNA
taatattcataaattgagcggc

SEQ ID NO: 59 - RNA, 22 nt, synthetic construct
  source 1..22: mol_type = other RNA
ttatatatgacagttgcttgag

SEQ ID NO: 60 - RNA, 22 nt, synthetic construct
  source 1..22: mol_type = other RNA
tctgagtaattcttcaccctgc

SEQ ID NO: 61 - RNA, 22 nt, synthetic construct
  source 1..22: mol_type = other RNA
taacttgatccaaagctctgag

SEQ ID NO: 62 - RNA, 22 nt, synthetic construct
  source 1..22: mol_type = other RNA
tatatatgacagttgcttgagt

SEQ ID NO: 63 - RNA, 22 nt, synthetic construct
  source 1..22: mol_type = other RNA
tcaggtgaactttgtctagtgc

SEQ ID NO: 64 - RNA, 22 nt, synthetic construct
  source 1..22: mol_type = other RNA
atgagtaaaatcaaacacactg

SEQ ID NO: 65 - RNA, 22 nt, synthetic construct
  source 1..22: mol_type = other RNA
taaaatcaaacacactgtgtcc

SEQ ID NO: 66 - RNA, 22 nt, synthetic construct
  source 1..22: mol_type = other RNA
aatattcataaattgagcggcc

SEQ ID NO: 67 - RNA, 22 nt, synthetic construct
  source 1..22: mol_type = other RNA
tttggcaagcatcctgtactgt

SEQ ID NO: 68 - RNA, 22 nt, synthetic construct
  source 1..22: mol_type = other RNA
tgaactttgtctagtgcttcca

SEQ ID NO: 69 - RNA, 22 nt, synthetic construct
  source 1..22: mol_type = other RNA
ataatattcataaattgagcgg

SEQ ID NO: 70 - RNA, 22 nt, synthetic construct
  source 1..22: mol_type = other RNA
tatgacagttgcttgagtttca

SEQ ID NO: 71 - RNA, 22 nt, synthetic construct
  source 1..22: mol_type = other RNA
ttggcaagcatcctgtactgtc

SEQ ID NO: 72 - RNA, 22 nt, synthetic construct
  source 1..22: mol_type = other RNA
tatattcctaaaataatgcttc

SEQ ID NO: 73 - protein, 11 aa, synthetic construct
  SITE 2..10: note = Intrachain disulfide bond
ACTGSTQHQCG